(19) United States
(12) Patent Application Publication
(10) Pub. No.: US 2023/0364229 A1
(43) Pub. Date: Nov. 16, 2023

(54) INTRATUMORAL AND SYSTEMIC IMMUNIZATION USING FRACTIONAL DAMAGE-CREATING DEVICE WITH CHECKPOINT MOLECULES FOR CANCER THERAPY

(71) Applicant: THE GENERAL HOSPITAL CORPORATION, Boston, MA (US)

(72) Inventors: Masayoshi KAWAKUBO, Malden, MA (US); Dieter MANSTEIN, Charlestown, MA (US)

(73) Assignee: THE GENERAL HOSPITAL CORPORATION, Boston, MA (US)

(21) Appl. No.: 18/030,427

(22) PCT Filed: Oct. 6, 2021
(86) PCT No.: PCT/US2021/053752

§ 371 Date: Apr. 5, 2023

Related U.S. Application Data
(60) Provisional application No. 63/088,281, filed on Oct. 6, 2020.

(51) Int. Cl. A61K 39/395; A61B 18/20; A61P 35/00

(52) U.S. Cl. CPC A61K 39/3955; A61B 18/203; A61P 35/00; A61B 2018/00577

(57) ABSTRACT

Provided herein are methods for inducing an anti-tumor immune response and/or treating cancer comprising treating tumor tissue of the subject with energy to induce fractional tissue damage in combination with one or more checkpoint molecule modulating agents.

### CROSS-REFERENCE TO RELATED APPLICATIONS

This application claims benefit under 35 U.S.C. § 119(e) to U.S. Provisional Application No. 63/088,281, filed Oct. 6, 2020, the contents of which are incorporated herein by reference in their entirety.

### FIELD OF THE INVENTION

The field of the invention relates to the treatment of cancer through induction of an anti-tumor immune response via treatment to induce fractional tissue damage.

### BACKGROUND

Fractional photothermolysis (FP), which generates a pattern of many microscopically small zones of thermal damage to tissue, is a widely used laser-assisted treatment modality. It is used for a large range of mainly dermatological indications, such as treatment of photodamaged skin, dyschromia, rhytides, and different kind of scars including acne, surgical and burn scars. Ablative fractional photothermolysis (aFP) uses a focused laser beam of a strongly absorbed wavelength that is directed to the tissue and thereby producing a pattern of microscopically small laser drills to the tissue. The central “hole” of each tissue lesion consists of physically-removed (ablated) tissue, surrounded by a small cuff of thermally-damaged tissue. In general, the width or diameter of these individual lesions, so called Microscopic Treatments Zones (MTZs) measures less than approximately 0.5 mm. aFP techniques typically expose only a small fraction of the tissue (often a treatment of approximately 5-20% of the surface area), leaving the majority of tissue spared or unexposed. Fractional tissue damage can be created using other energy sources including radiofrequency energy, focused ultrasound and the like and may extend from the surface into the tissue or may be entirely within the tissue in a fractional pattern or arrangement.

### SUMMARY

The compositions and methods described herein are based, in part, on the discovery that treating tumor tissue of the subject with energy to induce fractional tissue damage in combination with an immune checkpoint modulator(s) serves to induce an anti-tumor response (either locally or systemically) that is more robust than an anti-tumor response generated by the one or more immune checkpoint modulators alone.

Accordingly, provided herein in one aspect is a method for inducing an immune response in a subject in need thereof, the method comprising: (a) administering an inhibitor of a blocking checkpoint molecule and an agonist of a stimulative checkpoint molecule to a subject in need thereof, and (b) treating a tissue of the subject with energy to induce fractional tissue damage, wherein an immune response is increased compared to the immune response produced by the inhibitor of the blocking checkpoint molecule and the agonist of the stimulative checkpoint molecule in the absence of the fractional tissue damage.

In another embodiment of this aspect and all other aspects described herein, the fractional tissue damage induces CD8+ T cell recruitment and/or activation.

In another embodiment of this aspect and all other aspects described herein, the energy that induces fractional tissue damage is selected from laser energy, ionizing radiation, ultrasound, and radio frequency energy.

In another embodiment of this aspect and all other aspects described herein, the blocking checkpoint molecule is selected from the group consisting of: PD-1, PD-L1, CTLA4, B7-H3, B7-H4, VISTA, TMIGD2, B7-H7, BTLA, HVEM, CD160, LAG3, TIGIT, CD96, CD155, TIM-3, Galectin-9, Adenosine, Adenosine A2a receptor, IDO, TDO, CEACAM1, SIRP alpha, CD47, CD200R and CD200.

In another embodiment of this aspect and all other aspects described herein, the stimulative checkpoint molecule is selected from the group consisting of: OX40, 4-1BB, GITR, CD28, ICOS, LIGHT, CD27, DNAM-1, 2B4, DC-SIGN, DR3, and CD40.

In another embodiment of this aspect and all other aspects described herein, the blocking checkpoint molecule is PD-1 and the stimulative checkpoint molecule is OX40.

In another embodiment of this aspect and all other aspects described herein, the PD-1 inhibitor and/or the OX40 agonist comprises an antibody.

In another embodiment of this aspect and all other aspects described herein, the cancer is colon cancer, lung cancer, melanoma, or breast cancer.

In another embodiment of this aspect and all other aspects described herein, the laser energy is emitted from a fractional CO₂ laser.

In another embodiment of this aspect and all other aspects described herein, the immune response comprises a local and/or systemic response.

Another aspect provided herein relates to a method for inducing an anti-tumor immune response in a subject in need thereof, the method comprising: (a) administering an inhibitor of a blocking checkpoint molecule and an agonist of a stimulative checkpoint molecule to a subject having cancer, and (b) treating tumor tissue of the subject with energy to induce fractional tissue damage, wherein an anti-tumor immune response is increased compared to the anti-tumor immune response produced by the inhibitor of the blocking checkpoint molecule and the agonist of the stimulative checkpoint molecule in the absence of the fractional tissue damage.

Also provided herein, in another aspect, is a method for treating cancer in a subject in need thereof, the method comprising: (a) administering an inhibitor of a blocking checkpoint molecule, and an agonist of a stimulative checkpoint molecule to a subject having cancer, and (b) treating tumor tissue of the subject with energy to induce fractional tissue damage, thereby treating cancer in the subject.

In one embodiment of this aspect and all other aspects described herein, the energy that induces fractional tissue damage is selected from laser energy, ionizing radiation, ultrasound, and radio frequency energy.

In another embodiment of this aspect and all other aspects described herein, the energy that induces fractional tissue damage is laser energy.

In another embodiment of this aspect and all other aspects described herein, the blocking checkpoint molecule is selected from the group consisting of: PD-1, PD-L1, CTLA4, B7-H3, B7-H4, VISTA, TMIGD2, B7-H7, BTLA, HVEM, CD160, LAG3, TIGIT, CD96, CD155, TIM-3, Galectin-9, Adenosine, Adenosine A2a receptor, IDO, TDO, CEACAM1, SIRP alpha, CD47, CD200R and CD200.

In another embodiment of this aspect and all other aspects described herein, the stimulative checkpoint molecule is selected from the group consisting of: OX40, 4-1BB, GITR, CD28, ICOS, LIGHT, CD27, DNAM-1, 2B4, DC-SIGN, DR3, and CD40.

In another embodiment of this aspect and all other aspects described herein, the blocking checkpoint molecule is PD-1 and the stimulative checkpoint molecule is OX40.

In another embodiment of this aspect and all other aspects described herein, the PD-1 inhibitor and/or the OX40 agonist comprises an antibody.

In another embodiment of this aspect and all other aspects described herein, the cancer is colon cancer, lung cancer, melanoma, or breast cancer.

In another embodiment of this aspect and all other aspects described herein, the laser energy is emitted from a fractional CO₂ laser.

In another embodiment of this aspect and all other aspects described herein, the anti-tumor immune response or the treatment of cancer comprises induction of CD8+ T cells.

In another embodiment of this aspect and all other aspects described herein, the anti-tumor immune response comprises a systemic response.

In another embodiment of this aspect and all other aspects described herein, the anti-tumor immune response induces an abscopal effect against a tumor that is not treated with the fractional laser to induce fractional tissue damage.

In another embodiment of this aspect and all other aspects described herein, the anti-tumor immune response prevents or reduces the likelihood of cancer recurrence.

In another embodiment of this aspect and all other aspects described herein, the anti-tumor immune response increases progression-free survival, reduces the size of one or more tumors, and/or increases overall response rate.

In some embodiments, the tumor treated to induce fractional damage as described herein is at least 0.5 cm in one or more dimensions, e.g., at least 0.5 cm in diameter. In some embodiments, the tumor treated to induce fractional damage is at least 0.75 cm, 1.0 cm, 1.5 cm, 2.0 cm, 2.5 cm, 3.0 cm or 3.5 cm in one or more dimensions.

In some embodiments, the tumor treated to induce fractional damage is of a size indicating that it was initiated at that site at least two weeks prior to treatment to induce fractional damage, e.g., at least two weeks, one month, two months, three months, four months or more prior to treatment.

Another aspect described herein relates to a method for inducing an anti-tumor immune response in a subject in need thereof, the method comprising: (a) administering an OX40 agonist to a subject having cancer, and (b) treating tumor tissue of the subject with energy to induce fractional tissue damage, wherein the anti-tumor immune response is increased compared to the anti-tumor immune response produced by the OX40 agonist in the absence of the fractional tissue damage.

Also provided herein, in another aspect, is a method for treating cancer in a subject, the method comprising: (a) administering an OX40 agonist to a subject having cancer, and (b) treating tumor tissue of the subject with energy to induce fractional tissue damage, thereby treating cancer in the subject.

In another embodiment of this aspect and all other aspects described herein, the method further comprises administering an inhibitor of a blocking checkpoint molecule selected from the group consisting of: PD-1, PD-L1, CTLA4, B7-H3, B7-H4, VISTA, TMIGD2, B7-H7, BTLA, HVEM, CD160, LAG3, TIGIT, CD96, CD155, TIM-3, Galectin-9, Adenosine, Adenosine A2a receptor, IDO, TDO, CEACAM1, SIRP alpha, CD47, CD200R and CD200.

In another embodiment of this aspect and all other aspects described herein, the blocking checkpoint molecule is PD-1.

In another embodiment of this aspect and all other aspects described herein, the PD-1 inhibitor and/or the OX4 agonist comprises an antibody.

In another embodiment of this aspect and all other aspects described herein, the cancer is colon cancer, lung cancer, melanoma, or breast cancer.

In another embodiment of this aspect and all other aspects described herein, the energy that induces fractional tissue damage is selected from laser energy, ionizing radiation, ultrasound, and radio frequency energy.

In another embodiment of this aspect and all other aspects described herein, the fractional laser comprises a fractional CO₂ laser.

In another embodiment of this aspect and all other aspects described herein, the anti-tumor immune response or treatment of cancer comprises induction of CD8+ T cells, increase in number of CD8+ T cells, or activation of CD8+ T cells.

In another embodiment of this aspect and all other aspects described herein, the anti-tumor immune response or treatment of cancer comprises a systemic response.

In another embodiment of this aspect and all other aspects described herein, the anti-tumor immune response or treatment of cancer induces abscopal treatment of a tumor that is not treated with energy emitted from the fractional laser to induce fractional tissue damage.

In another embodiment of this aspect and all other aspects described herein, the anti-tumor immune response prevents or reduces the likelihood of cancer recurrence.

In another embodiment of this aspect and all other aspects described herein, the anti-tumor immune response increases progression-free survival, reduces the size of one or more tumors, and/or increases overall response rate.

In some embodiments, the tumor treated to induce fractional damage as described herein is at least 0.5 cm in one or more dimensions, e.g., at least 0.5 cm in diameter. In some embodiments, the tumor treated to induce fractional damage is at least 0.75 cm, 1.0 cm, 1.5 cm, 2.0 cm, 2.5 cm, 3.0 cm or 3.5 cm in one or more dimensions.

In some embodiments, the tumor treated to induce fractional damage is of a size indicating that it was initiated at that site at least two weeks prior to treatment to induce fractional damage, e.g., at least two weeks, one month, two months, three months, four months or more prior to treatment.

Another aspect provided herein relates to a system for inducing an anti-tumor immune response or treating cancer in a subject, the system comprising: a device configured to induce fractional tissue damage and means for administering an inhibitor of a blocking checkpoint molecule, and an agonist of a stimulative checkpoint molecule.

In one embodiment of this aspect and all other aspects described herein, the device configured to induce fractional tissue damage comprises a fractional laser, radiofrequency energy delivery device, or focused ultrasound device.

In another embodiment of this aspect and all other aspects described herein, the fractional laser is an ablative fractional laser.

In another embodiment of this aspect and all other aspects described herein, the fractional laser is a fractional CO₂ laser.

In another embodiment of this aspect and all other aspects described herein, the radiofrequency energy delivery device comprises an ablative fractional radiofrequency energy delivery device. A non-limiting example of a radiofrequency device that can be configured to deliver fractionally ablative radiofrequency energy includes the Candela™ Profound RF microneedling device. Similar devices are also contemplated for use with the methods and systems described herein.

Also described herein, in another aspect, is a method of inducing pyroptosis in tumor cells in a subject, the method comprising: (a) treating tumor tissue of a subject with energy to induce fractional tissue damage; and (b) administering an inhibitor of a blocking checkpoint molecule and an agonist of a stimulative checkpoint molecule to the subject, wherein pyroptosis is induced in tumor cells in the subject.

In one embodiment of this aspect and all other aspects described herein, pyroptosis is induced at a site separate from the tumor tissue treated with energy to induce fractional tissue damage.

Another aspect described herein relates to a method for inducing an anti-tumor immune response in a subject in need thereof, the method comprising: (a) administering one or more agents to a subject having cancer, and (b) treating tumor tissue of the subject with energy to induce fractional tissue damage, wherein the number of CD8+ T cells or activated CD8+ T cells is increased in the tumor compared to the anti-tumor immune response produced by the one or more agents in the absence of the fractional tissue damage.

Also provided herein, in another aspect, is a method for treating cancer in a subject, the method comprising: (a) administering one or more agents to a subject having cancer, and (b) treating tumor tissue of the subject with energy to induce fractional tissue damage, wherein the number of CD8+ T cells or activated CD8+ T cells is increased in the tumor, thereby treating the cancer in the subject.

### BRIEF DESCRIPTION OF THE FIGURES

FIGS. 1A-1D. Tumor Volume and Survival Curves after Treatment. To investigate whether aFP treatment could induce systemic anti-tumor immunity, a mouse model having one tumor on each hind leg was established with the aFP-treated tumor being on the left leg. The growth of both tumors was measured. aFP laser irradiation was performed 6 days after tumor inoculation. Anti-PD-1 inhibitor and OX40 agonist were administered intraperitoneally at a dose of 200 μg per mouse on days 6, 8, 10, 12 and 14, or 12, 14, 16, 18 and 20 after tumor cell inoculation. FIG. 1A, Average of tumor volume curves on the treated legs of mice which treated from day 6. FIG. 1B, Average of tumor volume curves on the untreated contralateral legs of mice which treated from day 6. FIG. 1C, Individual tumor volume curves on the untreated contralateral legs of mice in control, PD1+OX40 and aFP+PD1+OX40 groups. FIG. 1D, Kaplan-Meier survival curves of mice receiving tumor inoculation, which treated from day 6. The significance values for the difference between the survival curves are: Control vs. anti-PD-1: P=0.0007, Control vs. OX40: P<0.0001, Control vs. Anti-PD-1+OX40: P<0.0001, Control vs. aFP: P=0.0217, Control vs. aFP+anti-PD-1: P<0.0001, Control vs. aFP+OX40: P<0.0001, Control vs. aFP+anti-PD-1+OX40: P<0.0001, anti-PD-1 vs. OX40: n.s., anti-PD-1 vs. Anti-PD-1+OX40: P=0.001, anti-PD-1 vs. aFP: P=0.0281, anti-PD-1 vs. aFP+anti-PD-1: n.s., anti-PD-1 vs. aFP+OX40: n.s., anti-PD-1 vs. aFP+anti-PD-1+OX40: P<0.0001, OX40 vs. Anti-PD-1+OX40: P=0.0007, OX40 vs. aFP: P=0.0018, OX40 vs. aFP+anti-PD-1: n.s., OX40 vs. aFP+OX40: n.s., OX40 vs. aFP+anti-PD-1+OX40: P<0.0001, Anti-PD-1+OX40 vs. aFP: P<0.0001, Anti-PD-1+OX40 vs. aFP+anti-PD-1: P=0.01, Anti-PD-1+OX40 vs. aFP+OX40: P=0.0457, Anti-PD-1+OX40 vs. aFP+anti-PD-1+OX40: n.s., aFP vs. aFP+anti-PD-1: P<0.0001, aFP vs. aFP+OX40: P<0.0001, aFP vs. aFP+anti-PD-1+OX40: P<0.0001, aFP+anti-PD-1 vs. aFP+OX40: n.s., aFP+anti-PD-1 vs. aFP+anti-PD-1+OX40: P=0.0002, aFP+OX40 vs. aFP+anti-PD-1+OX40: P<0.0001.

FIGS. 2A-2D. Tumor volume and survival curves after treatment (days 12). To investigate whether a larger tumor can be cured by combination therapy such as anti-PD-1+OX40 agonist or aFP+anti-PD-1+OX40 agonist therapy, the treatment started 12 days after tumor inoculation. Anti-PD-1 inhibitor and OX40 agonist were administered intraperitoneally at a dose of 200 μg per mouse on days 12, 14, 16, 18 and 20 after tumor cell inoculation. FIG. 2A, Average of tumor volume curves on the aFP-treated legs of mice which treated from day 12. FIG. 2B, Average of tumor volume curves on the untreated contralateral legs of mice which treated from day 12. FIG. 2C, Individual tumor volume curves on the untreated contralateral legs of mice which treated from day 12. FIG. 2D, Kaplan-Meier survival curves of mice receiving tumor inoculation, which treated from day 12. The significance values for the difference between the survival curves are: Control vs. Anti-PD-1+OX40: P=0.0022, Control vs. aFP+anti-PD-1+OX40: P<0.0001, Anti-PD-1+OX40 vs. aFP+anti-PD-1+OX40: P=0.0252.

FIG. 3. Survival curves of rechallenge test. Kaplan-Meier survival curves of mice receiving the rechallenge test with CT26WT cells. These mice were each inoculated with 3.5×10⁵ CT26WT cells subcutaneously in the contralateral aFP-untreated thigh. The significance values for the difference between the survival curves are: control mice vs. survival mice in anti-PD-1+OX40 and aFP+anti-PD-1+OX40: P<0.0001.

FIGS. 4A-4F. Flow cytometric analysis for tumor infiltrating lymphocytes 5 days after aFP treatment in the aFP-untreated contralateral tumor. Flow cytometry analysis was performed 5 days after aFP treatment to investigate the number of CD3+, CD4+, and CD8+, granzyme B+CD8+ T cells and Tregs expressing CD4, and Foxp3 inside the untreated contralateral tumor. FIG. 4A, Proportion of CD3+ cells normalized to tumor weight. FIG. 4B, Proportion of CD4+ T cells normalized to tumor weight. FIG. 4C, Proportion of Treg normalized to tumor weight. FIG. 4D, Proportion of CD8+ T cells normalized to tumor weight. FIG. 4E, Proportion of granzyme B+CD8+ T cells normalized to tumor weight. FIG. 4F, Ratio of CD8+ T cells to Tregs (CD4+Foxp3+).

FIGS. 5A-5B. Flow cytometric analysis for dendritic cells (DCs) 3 days after aFP treatment in aFP-treated tumor and in drainage lymph node at aFP-treated side. Flow cytometry analysis was performed 3 days after aFP treatment to investigate the number of CD103+CCR7+DCs in aFP-treated tumor and XCR1+DCs in drainage lymph node at aFP-treated side. FIG. 5A, proportion of CD103+CCR7+DCs normalized to tumor weight. FIG. 5B, Absolute number of XCR1+DCs in drainage lymph node.

FIGS. 6A-6B. Flow cytometric analysis for OX40+Ki67+CD8+ T cells 5 days after aFP treatment in drainage lymph node at aFP-treated side. Flow cytometry analysis was performed 5 days after aFP treatment to investigate the number of OX40+Ki67+CD8+ T cells in drainage lymph node at aFP-treated side. FIG. 6A, Absolute number of OX40+Ki67+CD8+ T cells in drainage lymph node at aFP-treated side. FIG. 6B, Absolute number of PD-1+OX40+Ki67+CD8+ T cells in drainage lymph node at aFP-treated side.

FIGS. 7A-7B. Tumor survival curves after aFP+anti-PD-1+OX40 triple therapy with anti-CD8 depletion antibody. To investigate whether adaptive immunity is necessary to eradicate cancer cells after the triple therapy, tumor inoculation was performed with anti-CD8 depletion antibody to ablate CD8+ T cells in the mouse in the one tumor model. Anti-CD8 depletion antibodies were administered intraperitoneally at a dose of 200 μg per mouse every 3 days from one day before tumor inoculation to removal of mice as endpoint. The graph shows Kaplan-Meier survival curves of mice receiving tumor inoculation and anti-CD8 depletion antibody. The significance values for the difference between the survival curves are: anti-CD8+aFP vs. aFP+anti-PD-1+OX40: P=0.0018. FIG. 7A, shows tumor volume. FIG. 7B, shows mouse survival.

FIGS. 8A-8D. Flow cytometric analysis for DAMPs-expressing tumor and DCs expressing LRP1 3 days after aFP treatment in the aFP-treated tumor. Flow cytometry analysis was performed 3 days after aFP treatment to investigate percentage of HSP70, HSP90 and CALR-expressing GFP+ tumor and the number of LRP1+DCs in aFP-treated tumor. FIG. 8A, Percentage of HSP70-expressing cells of GFP+ cells. FIG. 8B, Percentage of HSP90-expressing cells of GFP+ cells. FIG. 8C, Percentage of CALR-expressing cells of GFP+ cells. FIG. 8D, Proportion of LRP1+DCs normalized to tumor weight.

FIG. 9. Experimental design scheme of in vivo

FIGS. 10A-10B. H & E stained histologic figures of tumor which was harvested immediately after the ablative fractional photothermolysis (aFP) laser treatment with a pulse energy of 100 mJ at a nominal density of 5%. FIG. 10A, Parallel view for direction of laser beam. FIG. 10B, Orthogonal view for direction of laser beam. White arrows indicate an ablated hole which is characteristic of aFP procedures. The ablated hole appeared to be collapsed and distorted within the tumor tissue.

FIG. 11. Dendritic cells stimulating CD8 killer T cells are induced in drainage lymph node 3 days after FP treatment with PD-1 inhibitor and OX40 agonist.

FIG. 12. Proliferating active CD8 killer T cells are induced in drainage lymph node and no treated contralateral tumor 5 days after FP treatment with PD-1 inhibitor and OX40 agonist.

FIG. 13. FP induces hyperactive DC and neutrophil activation. Fractional radio frequency (FRF) induces macrophage and neutrophil activation and pyroptosis of tumor cells. While the same device is used to produce fractional tissue damage (FP) and bulk FP ablation, the density is different: 5% (Normal FP) vs 25% (Bulk FP). For “Bulk Cryo,” cryo spray containing liquid nitrogen was used for 30 seconds and thawed using finger heat (repeated four times). For the FRF (fractional radiofrequency energy) group, the inventors used the following RF device and treatment parameters: Candela Profound RF microneedle device at 70 C and 5 sec.

FIG. 14. aFP+Anti-PD-1 inhibitor+OX 40 agonist combination therapy resulted in improved survival in mice having melanoma-based tumors as compared to mice having the same tumors but either untreated or treated with anti-PD-1 and OX40 agonist in the absence of fractional tissue damage.

FIG. 15. Extracted RNA quality from tumor sample. The ratio of 28s/18s RNA is an indicator of RNA quality. In bulk ablated tissues, 28s RNA is not detectable, indicating that the RNAs were destroyed due to strong heat.

FIG. 16. Exemplary electrograms from samples in control tissues, tissue with fractional damage or tissue with bulk ablation damage.

FIGS. 17A-17F. Exemplary fractional laser devices for use with the methods and systems described herein. FIG. 17A is a schematic of an exemplary fractional laser device. FIG. 17B is a top view of a first exemplary embodiment of a mask or shielding device for use in generating a pattern of fractional tissue damage at e.g., a tumor site. FIG. 17C shows a top view of a second exemplary embodiment of a mask or shielding device for use in generating a pattern of fractional tissue damage at e.g., a tumor site. FIG. 17D is an illustration of a second exemplary embodiment of a fractional laser device useful for the methods and systems described herein. FIG. 17E shows a top view of small individual exposure areas created by the fractional resurfacing system of FIG. 17D. FIG. 17F shows an exemplary embodiment of a system for monitoring the location of the fractional damaging system of FIG. 17D.

FIGS. 18A-18D. Exemplary fractional radiofrequency devices for use with the methods and systems described herein. FIG. 18A is a schematic drawing of a cross section of a tissue treated using an ASR method. FIG. 18B is a schematic drawing of a cross section of a tissue treated using an NSR method. FIG. 18C is a schematic illustration of an apparatus for inducing fractional tissue damage using electromagnetic energy according to one embodiment as described herein. FIG. 18D is a schematic illustration of portions of an apparatus for inducing fractional tissue damage according to one embodiment as described herein.

FIGS. 19A-19B show exemplary embodiments related to fractional tissue damage. FIG. 19A is an image depicting exemplary fractional tissue damage patterns (1 cm×1 cm) that can be used with the methods and systems described herein. FIG. 19B shows histology images depicting fractional tissue damage (at 5% density) as compared to bulk ablation techniques (at 25% density). The images show NBTC stained histology showing tissue viability (darker=viable, pale=non-viable) across various exposure parameters using an IPG ablative laser. The higher density, higher energy histology (lower right corner) shows no viable tissue while applying lower densities and lower energies leaves more viable tissue between the MTZs.

### DETAILED DESCRIPTION

Provided herein are compositions and methods useful for enhancing an immune response (e.g., an anti-tumor immune response) in combination with one or more immune checkpoint modulators. Such methods for enhancing an immune response can find utility in treating infections (e.g., chronic infections), or cancer.

In some embodiments, the immune response is an anti-tumor immune response. An enhanced anti-tumor immune response can reduce the tumor size or growth rate, prevent cancer or reduce the likelihood of recurrence following treatment of a primary tumor, and improve overall response rate. In addition, the induction of fractional tissue damage can improve the sensitivity of a given tumor to one or more immune checkpoint modulators (i.e., a tumor that was refractory to immune checkpoint modulation).

### Definitions

As used herein, the term “tumor” refers to a tissue, lesion or site comprising a plurality of cancer cells (e.g., at least two) and includes microscopic tumors. A tumor can be of any size, including for example, microscopic, at least 0.5 mm, at least 1 mm, at least 5 mm, at least 10 mm, at least 50 mm, at least 1 cm, at least 2 cm, at least 5 cm, at least 10 cm or more. A tumor can be a solid tumor (e.g., a sarcoma or carcinoma), a benign tumor, a malignant tumor, a primary tumor or a secondary or tertiary tumor site.

As used herein, the term “fractional tissue damage” generally describes the generation of damage, heating, and/or ablation/vaporization of multiple small individual exposure areas (e.g., “microscopic treatment zones”; generally having at least one dimension that is less than about 1 mm) of biological tissue or other tissue. Such damage can be produced by mechanical means or by exposing the tissue to energy, such as laser therapy (e.g., directed optical energy from a laser, such as a fractional laser), ionizing radiation, ultrasound, and/or radiofrequency energy. Fractional tissue damage retains regions of substantially undamaged, unablated, unfrozen, and/or unheated areas or volumes of tissue that are present between the irradiated, damaged, and/or ablated/vaporized regions (e.g., microtreatment zones). The individual exposure areas can be, for example, oval, circular, arced and/or linear in shape. The term “fractional tissue damage” specifically excludes bulk tumor ablation (e.g., damage of greater than 25% density over a given surface area) or excision. In some embodiments, the spots where the fractional energy is applied are non-overlapping or are discrete. In some embodiments, “fractional tissue damage” comprises damage of 0.1% to 24.5% of tissue at a tumor site; in other embodiments, the damage is limited to 0.1% to 20%, 0.1% to 15%, 0.1% to 10%, 0.1% to 5%, 0.1 to 1%, 20-24.5%, 15-24.5%, 10-25%, 5-24.5%, 1-15%, 5-15%, 10-15% of tissue at the tumor site or any integer therebetween.

The term “fractional” specifically excludes temporal fractional treatment and refers to damage produced at all desired microtreatment zones at a given clinical visit. That is, the term “fractional” specifically excludes damage that is partially induced over two or more office visits (e.g., treatment of a fraction of the target microtreatment zones during each visit). However, fractional tissue damage can be repeated as necessary, provided that all microtreatment zones are treated during each office visit.

“Bulk tissue ablation,” as that term is used herein, refers to damage of at least 25% tissue (inclusive) at the tumor or treatment site (e.g., at least 30%, at least 40%, at least 50%, at least 60%, at least 70%, at least 80%, at least 90% or complete ablation of the tumor (e.g., 100%).

The terms “nonablative” and “subablative” as used herein can refer to processes that cause tissue damage but do not involve vaporization or other energy-based removal of biological tissue or other material from the site of treatment at the time of treatment (e.g., bulk tissue ablation). Fractional tissue damage can be produced with both ablative and non-ablative devices and settings, provided that viable tissue is retained between damaged sites.

As used herein, the term “ablative fractional laser” refers to a device that removes tissue and leaves behind a hole surrounded by a coagulated cuff of tissue, whereas a “non-ablative fractional laser” refers to a device that coagulates but does not remove tissue. Exemplary devices and methods of generating fractional tissue damage are described in e.g., U.S. Pat. No. 9,351,792, the contents of which are incorporated herein by reference in their entirety.

As used herein, the term “ablative radiofrequency device” or “ablative focused ultrasound” refers to devices that produce thermal damage (akin to a non-ablative fractional laser) but do not necessarily remove tissue. An exemplary radiofrequency device and methods of generating fractional tissue damage using such a device are described in e.g., U.S. Pat. No. 9,570,899, the contents of which are incorporated by reference herein in their entirety.

As used herein, the term “microtreatment zone (MTZ)” refers to a discrete, non-overlapping spot to which energy is applied to induce damage at that site. Typically, an MTZ is less than 1 mm, for example, less than 0.75 mm, less than 0.5 mm, less than 0.4 mm, less than 0.3 mm, less than 0.25 mm, less than 0.1 mm, less than 0.09 mm, less than 0.08 mm, less than 0.07 mm, less than 0.06 mm, less than 0.05 mm, less than 0.04 mm, less than 0.03 mm, less than 0.02 mm or less than 0.01 mm.

As used herein, the term “immune checkpoint molecule” is used to refer to proteins that function to modulate the innate immune system. Such immune checkpoint molecules can regulate and maintain self-tolerance and the duration and amplitude of physiological immune responses. Immune checkpoint molecules can be described based on their ability to inhibit or stimulate the innate immune system. For example, immune checkpoint molecules that naturally inhibit innate immunity are referred to herein as “blocking checkpoint molecules.” In order to induce an anti-tumor immune response, one of skill in the art will recognize that inhibitors of such blocking checkpoint molecules will release the inhibition of the innate immune system produced by the blocking checkpoint molecule. Exemplary blocking checkpoint molecules include, but are not limited to, PD-1, PD-L1, CTLA4, B7-H3, B7-H4, VISTA, TMIGD2, B7-H7, BTLA, HVEM, CD160, LAG3, TIGIT, CD96, CD155, TIM-3, Galectin-9, Adenosine, Adenosine A2a receptor, IDO, TDO, CEACAM1, SIRP alpha, CD47, CD200R and CD200.

In contrast, an immune checkpoint molecule that can induce or increase an innate immune response is referred to herein as a “stimulative checkpoint molecule.” As will be appreciated by those of skill in the art, an agonist of a stimulative checkpoint molecule can induce or increase the innate immune response. Exemplary stimulative checkpoint molecules include, but are not limited to, OX40, 4-1BB, GITR, CD28, ICOS, LIGHT, CD27, DNAM-1, 2B4, DC-SIGN, DR3, and CD40.

The term “immune checkpoint modulator” refers to a molecule capable of modulating the function of an immune checkpoint protein in a positive or negative way (in particular the interaction between an antigen presenting cell (APC) such as a cancer cell and an immune T effector cell). The term “immune checkpoint” refers to a protein directly or indirectly involved in an immune pathway that under normal physiological conditions is important for preventing uncontrolled immune reactions and thus for the maintenance of self-tolerance and/or tissue protection. The one or more immune checkpoint modulator(s) in use herein may independently act at any step of T cell-mediated immunity including clonal selection of antigen-specific cells, T cell activation, proliferation, trafficking to sites of antigen and inflammation, execution of direct effector function and signaling through cytokines and membrane ligands. Each of these steps is regulated by counterbalancing stimulatory and inhibitory signals that fine tune the response. The term “immune checkpoint modulator” encompasses immune checkpoint modulator(s) capable of down-regulating at least partially the function of an inhibitory immune checkpoint (antagonist) and/or immune checkpoint modulator(s) capable of up-regulating at least partially the function of a stimulatory immune checkpoint (agonist). For example, the term “immune checkpoint modulator” can refer to molecules that may totally or partially reduce, inhibit, interfere with or modulate one or more blocking checkpoint proteins, which in turn regulates T-cell activation or function. Similarly, the term “immune checkpoint modulator” can also refer to molecules that can increase or induce the expression or activity of one or more stimulative checkpoint proteins, which in turn regulates T-cell activation or function. Immune checkpoint modulators include small molecules, peptides, peptidomimetics, and/or antibodies or antigen binding fragments thereof (e.g., a construct employing the antigen-binding domain of an antibody) that bind a checkpoint protein.

As used herein, the term “anti-tumor immune response” refers to an increase in immune cell-related activity within a tumor, lesion or tissue. Examples of an increased anti-tumor response include, but are not limited to, an increase in recruitment or number of inflammatory cells within the tumor or tissue, increased number of CD8+ T cells or activated CD8+ T cells, increased processing and presentation of released antigens by antigen-presenting cells (APCs) (e.g., CD103+ dendritic cells), increased interaction of tumor cells with T lymphocytes, increased immune/T-cell activation within or around the tumor, trafficking of antigen-specific effector cells to the tumor, or engagement of the target tumor cell by the activated effector T cell.

In some embodiments, the anti-tumor immune response is increased as assessed by measuring the number of CD8+ T cells within the tumor and can be increased by at least 20% in the presence of fractional tissue damage and one or more checkpoint modulators as compared to the number of CD8+ T cells in the presence of the one or more checkpoint modulators but the absence of fractional tissue damage.

As used herein, the term “systemic response” refers to an immune response that occurs distal to the site where the fractional tissue damage is generated. This is in contrast to a local immune response that occurs at the site where fractional tissue damage occurs. As an example, a systemic immune response can refer to an increase or induction in immune cell number or activity at a second, untreated tumor site, or in circulation (e.g., in the bloodstream). A systemic response permits abscopal treatment of a distal tumor (including microscopic tumors), immune cell activity against circulating cancer cells or contributes to or exacerbates an immune response induced by one or more immune checkpoint modulators.

As used herein, the term “induction of CD8+ T cells” refers to an increase in number or activity of CD8+ T cells within a tumor, lesion or tissue. In some embodiments, the methods and systems described herein can induce CD8+ cells or activated CD8+ T cells by at least 20%, at least 30%, at least 40%, at least 50%, at least 60%, at least 70%, at least 80%, at least 90%, at least 1-fold, at least 2-fold, at least 5-fold, at least 10-fold or more as compared to the number of CD8+ or activated CD8+ T cells induced with treatment to induce fractional tissue damage or one or more immune checkpoint inhibitors alone.

As used herein, the term “prevents or reduces the likelihood of cancer recurrence” refers to the ability of the immune system to retain the memory of certain antigens (e.g., cancer antigens) such that if the same or similar antigens are reintroduced or the cells with such cancer antigens begin to proliferate again, the immune system can activate an immune response quickly to remove the cancer cells and prevent further cancer recurrence. For example, if a given tumor is incompletely excised and some cancer cells remain and begin proliferating after treatment has ceased, or if a distal site of tumor cells arises, the immune system can rapidly mobilize to remove such cancer cells to prevent or reduce the likelihood of recurrence of the cancer. Typically, the term “prevents or reduces the likelihood of cancer recurrence” refers to the prevention of the same or a similar cancer sharing cancer antigens to the first incidence of cancer (i.e, the primary tumor).

The terms “decrease”, “reduced”, “reduction”, or “inhibit” are all used herein to mean a decrease or lessening of a property, level, or other parameter by a statistically significant amount. In some embodiments, “reduce,” “reduction” or “decrease” or “inhibit” typically means a decrease by at least 10% as compared to a reference level (e.g., the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased”, “increase” or “enhance” or “activate” are all used herein to generally mean an increase of a property, level, or other parameter by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, at least about a 20-fold increase, at least about a 50-fold increase, at least about a 100-fold increase, at least about a 1000-fold increase or more as compared to a reference level.

The term “pharmaceutically acceptable” can refer to compounds and compositions which can be administered to a subject (e.g., a mammal or a human) without undue toxicity.

As used herein, the term “pharmaceutically acceptable carrier” can include any material or substance that, when combined with an active ingredient, allows the ingredient to retain biological activity and is non-reactive with the subject's immune system. Examples include, but are not limited to, any of the standard pharmaceutical carriers such as a phosphate buffered saline solution, water, emulsions such as oil/water emulsion, and various types of wetting agents. The term “pharmaceutically acceptable carriers” excludes tissue culture media.

As used herein, the term “comprising” means that other elements can also be present in addition to the defined elements presented. The use of “comprising” indicates inclusion rather than limitation.

As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

Further, unless otherwise required by context, singular terms shall include pluralities and plural terms shall include the singular.

Other than in the operating examples, or where otherwise indicated, all numbers expressing quantities of ingredients or reaction conditions used herein should be understood as modified in all instances by the term “about.” The term “about” when used in connection with percentages can mean±1%.

### Induction of Fractional Tissue Damage

Essentially any method that induces fractional tissue damage, as that term is used herein, can be used with the methods and systems described herein including, but not limited to, laser therapy (e.g., directed optical energy from a laser, such as a fractional laser), non-laser optical energy, ionizing radiation, ultrasound (e.g., focused ultrasound), and radio frequency (RF) energy. For example, RF energy can be used to form a plurality of microscopic treatment zones in tissue using a plurality of surface or penetrating (e.g., needle-like) electrodes provided on the tissue surface and/or within the tissue to induce fractional damage of the tumor.

Such fractional damage can facilitate a local and/or systemic immune response, and/or promote an immune system attack on the tumor. It is also contemplated herein that fractional tissue damage can release tumor antigens that can in turn induce or complement an anti-tumor immunity. The study described in the working Examples also indicates that there is an improvement in the overall treatment of cancer when fractional damage is induced in the tumor over surgical approaches that excise the tumor. That is, in some embodiments it is more efficacious to treat the tumor to induce fractional damage than to excise the tumor surgically. Thus, in some embodiments, the method and systems described herein do not comprise surgical excision of the primary tumor.

In certain embodiments, fractional damage to tumor tissue can also enhance the efficacy of other therapies that can be used in combination. Thus in some embodiments, the dose of one or more therapies administered in combination with fractional laser treatment is lower than the dose of the one or more therapies required in the absence of fractional laser treatment (e.g., conventional anti-cancer treatment). Alternatively, fractional tissue damage produced as described herein can restore sensitivity of a tumor to an agent (e.g., an immune checkpoint modulator) to which the tumor was previously unresponsive or refractive to.

While well-suited to treatment of skin tumors, including but not limited to melanoma, fractional treatments can also be applied to tumors located elsewhere in the body (e.g., pancreatic cancer).

In some embodiments, methods and systems described herein utilize radiofrequency energy to induce fractional tissue damage. Thus, the methods described herein include providing to an individual in need thereof radiofrequency energy under sufficient conditions to induce an anti-tumor immune response (as that term is used herein), for example by using the application of a non-invasive radiofrequency field, including one generated by a radiofrequency signal between a transmission head and a reception head that is different from the transmission head. One can configure the transmission and reception heads on opposite sides of a desired target of the individual for treatment (such as a tumor site(s) or the whole body) and irradiate the site(s) between the transmission and reception heads with a radiofrequency field to kill, damage, or induce immune responses against the target cells from the interaction of the radiofrequency field with the cancer cells.

In specific embodiments, a non-invasive radiofrequency therapy system comprises a radiofrequency transmitter in communication with a transmission head and a radiofrequency receiver in communication with a reception head. The communication may be direct electrical, optical, and electromagnetic connections and indirect electrical, optical, and electromagnetic connections. That is, two devices are in communication if a signal from one is received by the other, regardless of whether the signal is modified by some other device. In an example of a non-invasive radiofrequency therapy system, the radiofrequency transmitter generates a radiofrequency signal at a frequency for transmission via the transmission head. In some cases, the radiofrequency transmitter has controls for adjusting the frequency and/or power and/or amplitude modulation of the generated radiofrequency signal and/or may have a mode in which a radiofrequency signal at a predetermined frequency and power are transmitted via the transmission head. In some cases, the radiofrequency transmitter provides a radiofrequency signal with variable amplitudes, pulsed amplitudes, multiple frequencies, etc.

In some embodiments, the microtreatment zones are each heated to between 37° C. and 45° C., for example. The target area may be heated to 37° C., 38° C., 39° C., 40° C., 41° C., 42° C., 43° C., 44° C., or 45° C. In specific embodiments, the radiofrequency power is determined by the type of system being employed. For example, for a portable system one may utilize 0-200 watts (W). In particular cases wherein the system is not portable, one may employ, e.g., from 700 W-1500 W to maintain a localized electric-field of strength 0-90 kV/m.

In certain embodiments, one or more particular wavelengths are employed. In specific cases, a frequency of 13.56 MHz is employed. Other examples are 1 MHz, 6.78 MHz, 8 MHz, 27.12 MHz, 40.68 MHz, 128 MHz, etc. In specific embodiments, a frequency range of 100 kHz to 1 GHz is employed. Other examples of ranges include 250 kHz-1 GHz, 500 kHz-1 GHz, 1000 kHz-1 GHz, 10,000 kHz-1 GHz, 100,000 kHz-1 GHz, 1 MHz-1 GHz, 10 MHz-1 GHz, 100 MHz-1 GHz, 500 MHz-1 GHz, 10 MHz-50 MHz, 10 MHz-100 MHz, 10 MHz-250 MHz, 10 MHz-500 MHz, and so forth.

In some embodiments, radiofrequency energy from the kilohertz to the low gigahertz range can cause effects in malignant tumor microenvironments, and these effects can be accentuated by using pulsed or amplitude modulated radiofrequencies.

The duration of exposure to radiofrequency energy may be of any suitable time, but in specific embodiments, it is on the order of minutes. In particular cases, the duration of exposure of the radiofrequency therapy for the individual is between 1-60, 1-50, 1-40, 1-30, 1-20, 1-10, 5-60, 5-50, 5-40, 5-30, 5-20, 10-45, 10-30, 10-20, 20-40, 20-30, 30-60, 45-60, 5-10, 5-9, 5-8, 5-7, 5-6, 6-10, 6-10, 6-8, 6-7, 7-10, 7-9, 7-8, 8-10, 8-9, or 9-10 minutes. The duration of the exposure of the RF therapy for the individual may be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 minutes in length. In cases wherein multiple exposures are provided to an individual, the different exposures may or may not be for the same duration in time.

Exemplary fractional radiofrequency devices: In one embodiment, fractional tissue damage can be achieved using a radiofrequency device and inserting an array of needles into a target region to a predetermined depth. Radio frequency pulses of electrical current are then applied to one or more of the needles, which can function as electrodes in monopolar or bipolar modes to create regions of thermal damage and/or necrosis in the tissue surrounding the tips of the needles (e.g., microtreatment zones). The tissue damage is achieved by delivering localized concentrations of electrical current that is converted into heat in the vicinity of the tips of the electrode needles.

In some embodiments, one or more of the needles in the array may be hollow and used to deliver small amounts of analgesic or anesthetic into the region being treated. These hollow needles may be interspersed among the electrode needles in the array, and they may also function as electrodes.

In another embodiment, the electrode needles may also be connected to a second source of electrical current in the milliampere range. Detection of a nerve close to any of the inserted needles of the array is achieved by sequential application of small currents to the needles in the array and observation of any visible motor response. If a nerve is detected, the nearby needle or needles can be deactivated during the subsequent application of RF current to other electrode needles in the array to avoid damaging the nerve.

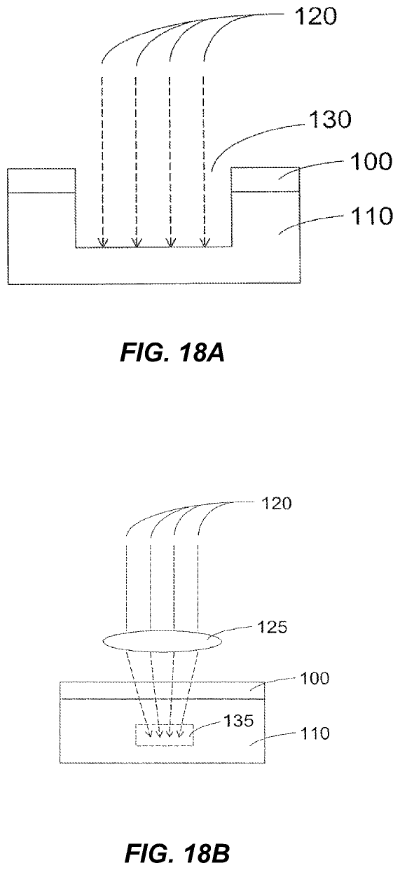

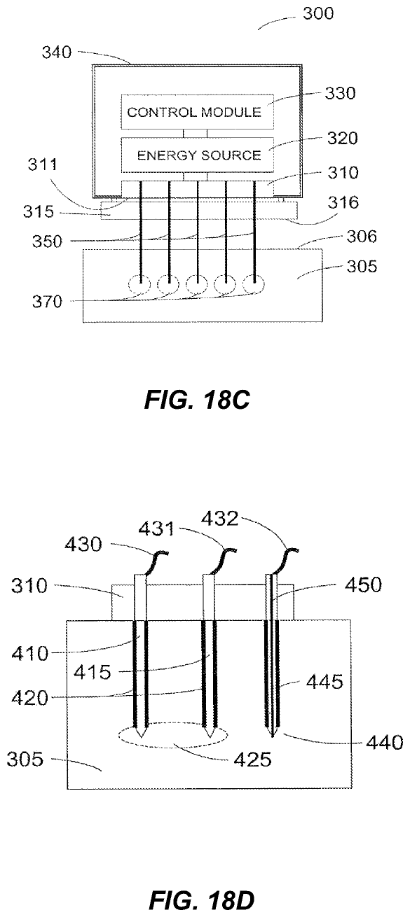

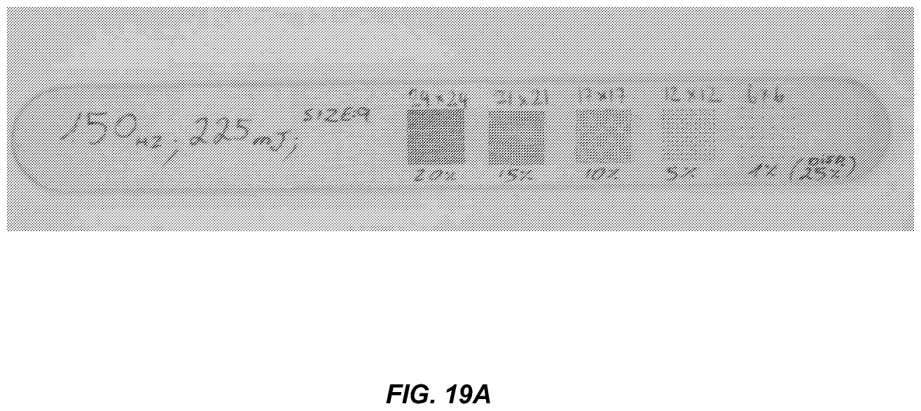

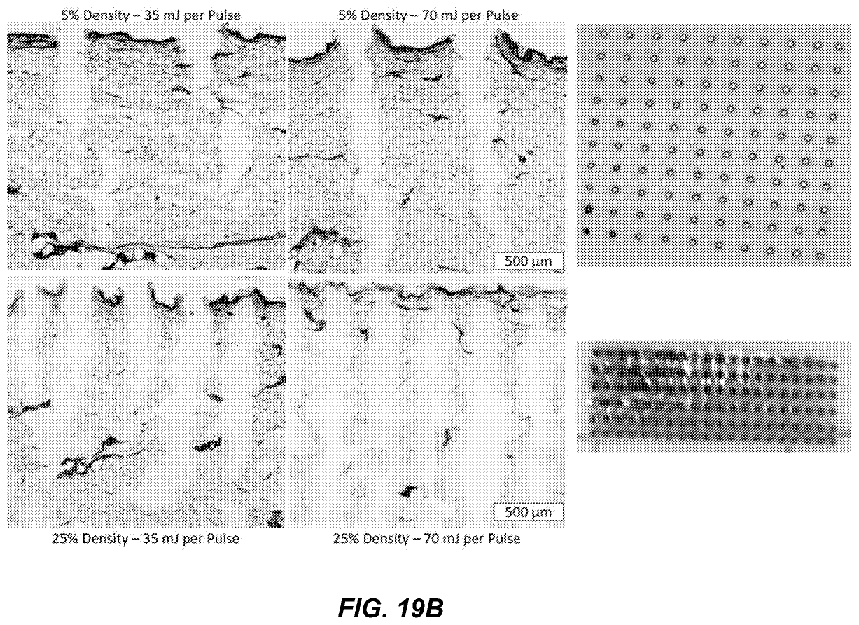

In an exemplary embodiment, a tissue treatment apparatus 300 as shown in FIG. 18 may be used to create regions of damage within the tissue being treated. The fractional damage apparatus can comprise a plurality of needles 350 attached to a base 310. The base is attached to housing 340 or formed as a part of the housing. A source of RF 21′ current 320 is electrically connected to each of the needles 350. A control module 330 permits variation of the characteristics of the RF electrical current, which can be supplied individually to one or more of the needles. Optionally, energy source 320 and/or control module 330 may be located outside of the housing.

In one exemplary embodiment, the energy source 320 is a radio frequency (RF) device capable of outputing signals having frequencies in a desired range. In another exemplary embodiment, the energy source is capable of outputting an AC or DC electric current. The control module 330 provides application-specific settings to the energy source 320. The energy source 320 receives these settings, and generates a current directed to and from specified needles for selectable or predetermined durations, intensities, and sequences based, on these settings.

In yet another embodiment, a spacer substrate 315 containing a pattern of small holes through which the array of needles protrudes may optionally be provided between the base 310 and the surface of the treatment area (e.g., tumor) 306. This spacer substrate can be used to provide mechanical stability to the needles. Optionally, this substrate can be movably attached to the base 310 or housing 340 and adjustable with respect to base 310, supporting the array of needles to control the depth of the needles protruding from the lower surface 316 of spacer substrate 315, and thus controlling the depth to which the needles are inserted into the skin or tumor.

In using such a device for inducing fractional tissue damage, the sharp distal ends of needles 350 pierce the surface 306 of the tissue 305 and are inserted into the tissue until the bottom surface 316 of spacer substrate 315 (or the bottom surface 311 of base 310 if a spacer substrate 315 is not used) contacts the surface 306 of the tissue or tumor 305. This configuration permits reliable insertion of the array of needles to a predetermined depth within the tissue being treated. Control module 330 is then configured to deliver controlled amounts of RF current to one or more needles 350.

Base 310 and/or spacer substrate 315, if used, can be planar or they can have a bottom surface that is contoured to follow the shape of the region of tissue being treated. This permits penetration of the needle array to a uniform depth within the targeted tissue even if the surface of the target area is not planar, e.g., along the eye sockets.

In another embodiment, base 310 and/or a spacer substrate 315, if used, can be cooled by any suitable means (such as by embedded conduits containing circulating coolant or by a Peltier device) to cool the surface of the skin when the needle array penetrates the skin or tumor tissue to reduce or eliminate pain. The surface region of the tumor being treated and/or the needles themselves may also be precooled by separate means, including convective or conductive means, prior to penetration of the skin or tumor tissue by the array of needles.

In some embodiments, the shafts of conductive needles 350 are electrically insulated except for the portion of the needle near the tip. In the apparatus of FIG. 18, application of RF current to the needles 350 causes heating in the exposed tip region, inducing thermal damage regions 370 around the tip of each needle. Thermal damage regions 370 result from operation of the apparatus in monopolar configuration, in which a remote grounding electrode, not shown in FIG. 18, is attached to a remote part of the patient's body to complete the circuit of electricity conveyed to needles 350 by energy source 320. In this monopolar configuration, RF current causes heating of the tip regions of the needles 350, generating thermal damage in tissue regions 370 adjacent to the needle tips that are approximately spherical or slightly elongated in shape.

In one embodiment, the current can be delivered simultaneously to all needles in the array to produce a pattern of thermal damage around the tip of each needle. In alternative embodiments, control module 330 and energy source 320 can be configured to supply electrical current to individual needles, to specific groups of needles within the array, or to any combination of individual needles in any desired temporal sequence. Providing current to different needles at different times during treatment (instead of heating all needles in the array at once) may help to avoid potential local electrical or thermal interactions among needles that can lead to excessive local damage and permits the retention of viable tissue between each microtreatment zone.

In some embodiments one or more vibrating means, such as a piezoelectric transducer or a small motor with an eccentric weight fixed to the shaft, may be mechanically coupled to housing 340 and/or base 310 that supports the array of needles 350. Vibrations conductively induced in needles 350 by such vibrating means can facilitate the piercing of the tissue by the needle tips and subsequent insertion of the needles into the tissue. The vibrating means can have an amplitude of vibration in the range of about 50-500 μm or, more preferably, between about 100-200 μm. The frequency of the induced vibrations can be from about 10 hz to about 10 khz, more preferably from about 500 hz to about 2 khz, and even more preferably about 1 khz. The particular vibration parameters chosen can depend on the size and material of the needles, the number of needles in the array, and the average spacing of the needles. The vibrating means can further comprise an optional controller capable of adjusting the amplitude and/or frequency of the vibrations.

Additional details and embodiments are shown in FIGS. 18A-18D. Conductive needles 410 and 415 are shown attached to base 310. Insulation 420 covers the shaft of needles 410 and 415 protruding from base 310 except for the region near the lower tip, and electrically insulates each conductive needle shaft from surrounding tissue 305. Electrical conductors 430 and 431, which may be wires or the like, extend from an upper portion of needles 410 and 415 respectively, and are connected to the energy source (not shown here). Suitable insulating materials for insulation 420 include, but are not limited to, Teflon®, polymers, glasses, and other nonconductive coatings. A particular material can be chosen as an insulator to facilitate penetration and insertion of needles 410 and 415 into tissue 305.

Needles 410 and 415 are shown operating in bipolar mode in another embodiment. Needle 410 is a positive electrode delivering RF or other current to the tip region of the needle from the energy source via conductor 430. Needle 415 functions as a grounding electrode that is connected to the ground of the energy source via conductor 431. In this configuration the applied current will travel through the tissue between the tips of needles 410 and 415, generating an elongated region of thermal damage 425 around and between the tips of the two needles.

An elongated region of damaged tissue 425 can be created between any two adjacent or nearby needles in the array through proper configuration of control module 330 and energy source 320. In one embodiment, elongated damage regions 425 are formed between several pairs of needles within the array of needles to form a desired damage pattern in the tissue 305. The regions of thermal damage 325 created in bipolar operation of the apparatus can be formed simultaneously or, alternatively, sequentially, using any combinations of proximate needles in the array to form each region. A wide variety of thermal damage patterns can be created using a single array of needles through appropriate configuration of energy source 320 and control module 330 to deliver predetermined amounts of current between selected pairs of needles. This apparatus thus allows for the creation of complex damage patterns within the tissue 305.

In practicing the methods and apparatus as described herein, the needles can have a width of about 500-1000 μm or preferably about 700-800 μm. Needles less than 500 μm in diameter can also be used if they are mechanically strong enough. Needles thicker than about 1000 μm in diameter may be undesirable because of the difficulty in forcing larger needles to penetrate the tissue and because of the increased propensity for pain and scarring. The length of the needles extending into the depend on the targeted depth for damaging the tissue. A typical depth can be about 1500-2000 μm, although shallower or deeper distances may be preferred for different treatments and regions of the body being treated. Needles within a single array may protrude by different lengths from the base 310 or spacer substrate 315. This will cause the tips of the needles to be positioned at different depths within the tissue being treated, and allow creation of damaged tissue at more than one depth. This variation in needle depth can achieve formation of damaged tissue over a larger volume within the tissue being treated.

The needle arrays can have any geometry appropriate for the desired treatment being performed. The spacing between adjacent needles is preferably greater than about 1 mm apart, and may be as large as about 2 cm. The spacing between needles in an array need not be uniform, and can be closer in areas where a greater amount of damage or more precise control of damage in the target area of tissue is desired. In one embodiment, the array of needles can comprise pairs of needles separated from adjacent pairs by larger distances. This geometry may be well-suited for inducing damage in bipolar mode between pairs of needles. Needles can also be arranged in a regular or near-regular square or triangular array. In any array geometry, the pattern of damage can be controlled with some precision by adjusting the intensity and duration of power transmitted to single needles or pairs of needles.

The amount of energy directed to a given needle will vary depending on the tissue being treated and the desired extent of thermal damage to induce. For typical needle spacings noted above, the energy source should be configured to deliver about 1-100 mJ per needle or pair of needles in the array. It may be preferable to initially use lower amounts of energy and perform two or more treatments over the same target area in a session to better control the damage patterns.

In yet another embodiment, one or more of the needles in the array may be hollow, such as needle 440 in FIG. 18. Center channel 450 can be used to deliver a local analgesic such as lidocaine 2% solution from a source (not shown) located within or above base 310 into the tissue 305 to reduce or eliminate pain caused by the thermal damage process.

In other embodiments, a hollow needle 440 is bifunctional, capable of conducting RF current or other energy via conductor 432 and also capable of delivering a local analgesic or the like through center channel 450. Similar to needles 410 and 415, bifunctional needle 440 has insulation 445 covering the shaft extending from base 310 except for the region near the lower tip. Analgesic may be supplied to the tissue either before or during application of RF or other current to the needle 450.

In other embodiments, one or more of the needles in the array can be bifunctional like needle 440. Alternatively, one or more needles can be hollow and optionally nonconductive, suitable only for delivering a local analgesic or the like. The array of needles used for a given application can comprise any combination of solid electrodes, bifunctional needles, or hollow nonconductive needles. For example, one type of needle array can comprise pairs of electrode needles operating in bipolar mode, with a hollow needle located between each pair. In this configuration, the hollow needle can deliver analgesic to the tissue between the tips of the electrode needles prior to applying current to the electrodes and causing thermal damage in the numbed tissue.

In other embodiments, one or more of the needles in the array can be further connected to an electronic detection apparatus and perform the additional flux-lion of a probe to detect the presence of a nerve near the tip. The electronic detection apparatus can comprise a source of electrical current in the milliampere range and control means to send small currents on the order of a milliamp to specific needles in the array. Detection of a nerve close to any of the inserted needles of the array is performed by sequential application of small currents to the needles in the array and observation of any visible motor response. If a nerve is detected, control module 330 can be configured to deactivate the needle or needles close to the nerve during the subsequent treatment to avoid damaging the nerve. A nerve detection method based on principles similar to those described herein is disclosed by Urmey et al. in Regional Anesthesia and Pain Medicine 27:3 (May-June) 2002, pp. 261-267.

In another embodiment, one or more of the needles can be hollow, and a light fiber or light guide is inserted into the hollow needle such that one end of it extends to or slightly protrudes from the needle tip. The other end of the light fiber or light guide is in communication with a source of optical energy. Optical energy supplied to the tip of the light guide or light fiber can then be used to heat the tip, which then heats the surrounding tissue, the target area, to cause fractional wounding at the needle tip. An array of needles used in accordance with the present invention can comprise a mix of electrode needles and light-guide needles. Alternatively, each needle can carry a light guide and all of the energy used to cause thermal damage can be generated by the optical energy source instead of using RF or other electrical current. A portion of the light guide or light fiber, such as the portion at the tip of the needle, can be configured to absorb energy and facilitate conversion of the optical energy to heat. In these embodiments, the optical energy source can comprise, but is not limited to, a diode laser, a diode-pumped solid state laser, an Er:YAG laser, a Nd:YAG laser, an argon-ion laser, a He—Ne laser, a carbon dioxide laser, an eximer laser, or a ruby laser. The optical energy conveyed by a light guide or light fiber can optionally be continuous or pulsed.

### Fractional Laser Treatment

In some embodiments, the means for producing fractional tissue damage comprises the use of a fractional laser. In certain embodiments, the fractional damage can be generated using an ablative fractional photothermolysis (aFP) procedure. Unlike conventional ablative treatments of tumors, which are directed to complete destruction or bulk tumor ablation of the tumor tissue using a laser or other optical energy source, fractional tissue damage (e.g., using a fractional laser treatment) involves the generation of a large number of small, discrete treatment zones within a region of the tumor tissue. Accordingly, a region or volume of tissue (e.g., tumor tissue) treated during an aFP procedure, will exhibit a number of discrete microscopic treatment zones (MTZs) where the tissue has been altered (e.g., partially or fully ablated or vaporized) by the laser radiation. These MTZs will be present within a larger volume of tissue that remains substantially unaltered by the laser radiation.

Fractional laser parameters can be optimized to achieve a desired degree of fractional tissue damage in a given tissue or tumor. These treatment parameters can include, for example, wavelength, local irradiance, local fluence, pulse energy, pulse duration, treatment zone size or spot size, treatment zone density, beam diameter, and combinations thereof. Laser energy can be applied internally, e.g., via catheter or during surgery.

For example, the number and density of MTZs can be predetermined by selecting desired fractional treatment parameters. In certain embodiments, the fractional treatment can be performed by directing a beam of energy onto a plurality of locations on the surface of the tissue (e.g., tumor tissue) being treated. In further embodiments, a plurality of beams can be directed simultaneously onto a plurality of locations on the tissue surface. The plurality of beams can be provided by a plurality of lasers or laser diodes, or alternatively by splitting a single beam of energy into a plurality of beams using an optical arrangement.

It is contemplated herein that the multiple microtreatment zones can be exposed sequentially or simultaneously. Sequential exposure can be achieved by scanning or moving an energy source which may be either pulsed, shuttered or continuous. Simultaneous exposure can be achieved, for example, by an array of sources or a multi-array of lenses. The array of sources can be a uni-dimensional array, a bi-dimensional array or the like. The array can be moved relative to the target area, and one or multiple passes of treatment can be performed in a target area (e.g., a tumor).

In some embodiments, fractional treatment of tumor tissue can provide an areal fraction of tissue surface that is irradiated that is between about 0.1% to 50% of the tumor (or preferably 0.1% to 24.5% of the surface area). Such smaller fractions of treated tissue can better avoid overall bulk heating of the tumor tissue while generating local damage therein and inducing an immune response. For a particular beam diameter, this areal fraction can be determined as the area of an individual beam cross-section multiplied by the number of distinct beam irradiation locations on a treated surface region, divided by the area of the treated surface region. Similar calculations of areal coverage can be determined, e.g., for different beam shapes and irradiation geometries including, e.g., irradiation patterns that include ellipses, thin lines, etc. by dividing the total area of irradiating energy beams directed onto the treated region by the area of the treated region.

In another embodiment of this aspect and all other aspects provided herein, the fractional laser induces fractional damage of at least 0.5% of the tumor volume, e.g., at least 1%, at least 1.5%, at least 2%, at least 2.25%, at least 2.5%, at least 3%, at least 3.5%, at least 4%, at least 5%, at least 10%, at least 15%, at least 20%, at least 25%, at least 30%, at least 40%, or at least 50% of the tumor volume. In another embodiment of this aspect and all other aspects provided herein, the fractional laser induces fractional damage of less than 0.5% of the tumor volume, e.g., less than 1%, less than 1.5%, less than 2%, at less than 2.25%, less than 2.5%, less than 3%, less than 3.5%, less than 4%, less than 5%, or less than 10%, less than 15%, less than 20%, less than 25%, less than 30%, less than 40%, or less than 50% of the tumor volume, provided that viable tissue is retained between each of the spots where the fractional energy is applied.

In some embodiments, the individual energy beams (which may be pulsed) that are used to create the MTZs in tissue can be generally less than 1 mm in width or diameter. Such width approximately corresponds to the width of the MTZs formed by the beams, and can be well-tolerated by the surrounding tissue and can prevent excessive or widespread disruption of the tumor tissue that could lead to spreading of tumor cells within the patient. In further embodiments, the width of these beams can be less than 0.5 mm, or less than 0.2 mm. Such smaller beam widths can generate MTZs that are narrow enough to disrupt tumor tissue and induce an anti-tumor immune response, while further reducing the likelihood of unwanted spreading or ‘release’ of tumor cells within the patient. The MTZs can be formed as ablated holes within the tissue, which may partially or completely collapse soon after formation.

The depth of the ablated holes and/or of the MTZs formed during fractional treatment of tumor tissue can be determined using known techniques based on, e.g., the wavelength(s) of energy used, the fluence, cross-sectional area and power of the energy beams, the characteristics of the treated tissue, etc. In general, it is preferable that the MTZs extend to one or more particular depths within the tumor tissue. For example, in certain embodiments, the MTZs can extend to a depth that is at least about ¼ of the distance between the tumor surface and the center of the tumor. The particular depth(s) of the MTZs can be selected based on the size and type of tumor being treated. For example, the depth of the MTZs can be selected such that they extend through an outer layer of the tumor and at least into an interior (or core) region of the tumor. In still further embodiments, characteristics of the fractional treatment can be selected such that the MTZs (e.g., ablated holes) can extend completely through the entire tumor. In some embodiments, the fractional laser penetrates to a depth of at least 0.1 mm (e.g., at least 0.2 mm, at least 0.3 mm, at least 0.4 mm, at least 0.5 mm, at least 1 mm, at least 1.5 mm, at least 2 mm, at least 2.5 mm, at least 3 mm, at least 3.5 mm, at least 4 mm, at least 4.5 mm, at least 5 mm etc.) into the tumor tissue.

In further embodiments, one or more tumors being treated can be located below another exposed tissue surface, such as a skin tissue. The parameters of the fractional treatment can be selected such that the MTZs extend through the overlying tissue, and into or through the tumor as described above. Conventional calculations using known energy and tissue parameters can be performed by one of ordinary skill in the art to provide a set of parameters for the applied energy (e.g., beam width, duration, wavelength, fluence, power, etc.) for specific procedures in accordance with the present disclosure, e.g., to generate MTZs that extend a particular depth into tumor and/or overlying tissue.

In still further embodiments, tumors located within the body (e.g., away from an exposed tissue surface) can also be treated. For such tumors, fractional treatment can be performed by delivering energy to the tumor(s) using a fiberscope, an endoscope, a catheter-disposed arrangement configured to deliver energy, a laparoscopic device, focused ultrasound energy device, or the like. In such embodiments, the energy (beam) parameters can be selected to produce MTZs within the tumor tissue as described above.

In certain embodiments, a CO₂ laser can be used to form the MTZs during fractional treatment of tumor tissue. In further embodiments, the energy source can be an erbium laser (e.g., an Er:YAG laser), or another type of laser capable of ablating biological tissue.

In some embodiments, fractional damage of tumor tissue can be performed non-ablatively, to generate MTZs of intact but thermally-damaged tissue within the tumor. Such non-ablative FP can be performed using an energy source such as, e.g., a pulsed dye laser, a Nd:YAG laser, or an Alexandrite laser. In still further embodiments, MTZs of non-ablative fractional damage can be generated in tumor tissue using focused ultrasound energy having a sufficiently low intensity to avoid ablation of tissue.

In still further embodiments, MTZs can be formed in tumor tissue by generating mechanical damage, e.g., by piercing the tumor tissue with an array of needles or multiple times with a single needle. A diameter of the needles can be less than about 1 mm, e.g., less than 0.5 mm, or about 0.1 to 0.2 mm. In certain embodiments the needle(s) can be heated prior to insertion into the tumor tissue to produce some thermal damage as well as mechanical disruption. For example, the needle(s) can be heated using a heated bath or other hot reservoir, or by providing a controlled amount of radiofrequency (RF) energy to the needle(s).

Because of the small size of the MTZs formed during aFP and other fractional procedures, tissue damage produced in the MTZs is well-tolerated, and can induce a healing response in surrounding healthy tissue. Such effects have been observed in dermatological applications of various types of fractional treatment.

The MTZ sizes (e.g., widths and depths) described herein can facilitate limited exposure of the interior of the tumor to the body's immune system and thereby stimulate or activate an immune response. For example, histology performed following aFP treatments of certain tumor tissues revealed an elevated level of erythrocytes, indicating an enhancement of blood flow within the tumor resulting from the aFP treatment. The apparent increase in blood flow in the tumor can facilitate some limited transport of tumor cells out of the tumor, but can also facilitate access of immune competent cells to the core region of the tumor. For example, the enhanced tissue pressure within the core of rapidly-growing tumors can make the core region inaccessible to immune competent cells, which rely on vascular perfusion of the tumor. Also, despite their observed collapse, and without wishing to be bound by theory, ablated channels (e.g., MTZs) in tumor tissue can facilitate access of immune competent cells to cancer cells within the tumor.

Ablative FP CO₂ laser treatments produce small holes in tissue by vaporization thereof at temperatures exceeding 100° C. This results in a steep temperature gradient surrounding the individual MTZs that include the vaporized holes. This steep temperature gradient exposes tumor cells adjacent to the laser-induced holes to a range of temperatures ranging from the peak temperature down to normal body temperature. Accordingly, without being bound by theory, fractional treatment of tumor tissue using aFP or other energy-based techniques (including, e.g., mechanical damage accompanied by local heating, as can be achieved with insertion of heated needles into tumor tissue) can produce weakened (e.g., thermally-damaged) tumor cells and also facilitate their exposure to components of the body's immune system. Such exposure can facilitate an immune response and/or other responses to the cancerous tissue without ‘overwhelming’ the body's defenses or allowing a large number of active tumor cells to spread through the body after such fractional treatment. In some embodiments, treatment of a tumor with ablative FP is performed using settings that do not cause substantial loss of immune cells in the tumor.

Exposure of cells surrounding the MTZs to a range of temperatures can occur without significant bulk heating in the fractionally-treated tissue volume, indicating a lack of confluent thermal injury within the tumor tissue. This particular thermal injury pattern within the tumor tissue distinguishes aFP treatment of tumor tissue from prior energy-based tumor treatment approaches using physical modalities, such as ionizing radiation therapy or classical thermal ablation approaches, that typically provide a relatively homogenous dose of energy throughout the tumor tissue.

Accordingly, one possible advantage of the thermal damage pattern characteristic of FP treatments is that throughout the tumor, cancer cells are exposed to a range of temperatures that can vary from the normal body temperature of the host up to the vaporization temperatures generated in the MTZs, which may be in excess of 100° C.

Also provided herein, in other aspects, are methods for treating cancer in a subject, for example, a method comprising: contacting tissue of a tumor with a fractional laser, thereby treating cancer in the subject. In one embodiment of this aspect and all other aspects provided herein the method for treating cancer does not comprise substantial ablation or removal of tissue from the tumor (i.e., less than 5% of the total tumor tissue is ablated/removed; less than 4%, less than 3.5%, less than 3%, less than 2.5%, less than 2.25%, less than 2%, less than 1.75%, less than 1.5%, less than 1.25%, less than 1%, less than 0.5% or less).

In one embodiment of this aspect and all other aspects provided herein, the fractional laser is a CO₂ laser. In one embodiment of this aspect and all other aspects provided herein, the parameters of the fractional laser are tuned such that the laser is non-ablative.

In another embodiment of this aspect and all other aspects provided herein, the fractional laser penetrates to a depth of at least 0.1 mm (e.g., at least 0.2 mm, at least 0.3 mm, at least 0.4 mm, at least 0.5 mm, at least 1 mm, at least 1.5 mm, at least 1.75 mm, at least 2 mm, at least 2.25 mm, at least 2.5 mm, at least 3 mm, at least 3.5 mm, at least 4 mm, at least 4.5 mm, at least 5 mm, etc.) into the tumor tissue.

In another embodiment of this aspect and all other aspects provided herein, treatment with the fractional laser induces a local immune response in the tumor tissue.

In another embodiment of this aspect and all other aspects provided herein, treatment with the fractional laser does not damage the stratum corneum. In another embodiment of this aspect and all other aspects described herein, the fractional laser treatment does not result in substantial ablation or removal of tissue from the tumor (i.e., less than 5% of the total tumor tissue is ablated/removed).

In another embodiment of this aspect and all other aspects provided herein, treatment with the fractional laser does not induce scarring or crusting of the tumor tissue.

In another embodiment of this aspect and all other aspects provided herein, the area of treatment comprises at least 0.25 mm². In other embodiments of this aspect and all other aspects provided herein, the area of treatment is at least 0.25 mm² up to and including the entire surface of a lesion. In other embodiments of this aspect and all other aspects described herein, the area of treatment comprises at least 5% of the tumor or lesion area; in other embodiments the area of treatment comprises at least 10%, at least 15%, at least 20%, at least 25%, at least 30%, at least 40%, at least 50%, or more of the tumor or lesion area. In certain embodiments, fractional tissue damage is limited to less than 60% of the tumor or lesion (i.e., less than 50%, less than 40%, less than 30%, less than 20%, less than 20%, less than 15%, less than 10%, or less than 5% of the tumor or lesion).

In another embodiment of this aspect and all other aspects provided herein, the energy of the fractional laser is 1 mJ to 200 mJ. In another embodiment of this aspect and all other aspects described herein, the energy of the fractional laser is in the range of 1 mJ to 5 mJ, 1 mJ to 10 mJ, 1 mJ to 20 mJ, 1 mJ to 30 mJ, 1 mJ to 40 mJ, 1 mJ to 50 mJ, 1 mJ to 75 mJ, 1 mJ to 100 mJ, 1 mJ to 125 mJ, 1 mJ to 150 mJ, 1 mJ to 175 mJ, 25 mJ to 200 mJ, 50 mJ to 200 mJ, 100 mJ to 200 mJ, 125 mJ-200 mJ, 150 mJ to 200 mJ, 175 mJ to 200 mJ, 50 mJ to 100 mJ, 25 mJ to 75 mJ, 25 mJ to 150 mJ, or any range therebetween. In another embodiment of this aspect and all other aspects provided herein, 40-60 mJ (e.g., 50 mJ) of energy is used for a superficial lesion and 150-200 mJ (e.g., 200 mJ of energy) is used for a deep tumor. In one embodiment, 100 mJ of energy is used for the superficial or deep lesion.

In another embodiment of this aspect and all other aspects provided herein, the pulse duration of the fractional laser is 100 μsec to 10 msec. In another embodiment of this aspect and all other aspects provided herein, the pulse duration of the fractional laser is 2 msec. In other embodiments of this aspect and all other aspects provided herein, the pulse duration of the fractional laser is between 100 μsec to 5 msec, 100 μsec to 1 msec, 100 μsec to 500 μsec, 100 μsec to 250 μsec, 100 μsec to 200 μsec, from 250 μsec to 10 msec, from 500 μsec to 10 msec, from 750 μsec to 10 msec, from 1 msec to 10 msec, from 2 msec to 10 msec, from 5 msec to 10 msec, from 1 msec to 5 msec, from 1 msec to 3 msec or any range therebetween.

In another embodiment of this aspect and all other aspects provided herein, the spot size of the fractional laser is 10 μm to 1 mm. In other embodiments of this aspect and all other aspects provided herein, the spot size of the fractional laser is in the range of 10 um to 750 μm, 10 um to 500 um, 10 um to 250 um, 10 um to 150 um, 10 um to 100 um, 10 um to 50 um, 10 um to 25 um, 400 um to 1 mm, 500 um to 1 mm, 600 um to 1 mm, 700 um to 1 mm, 800 um to 1 mm, 900 um to 1 mm, 50 um to 750 um, 75 um to 500 um, 100 um to 500 um, 250 um to 500 um, or any range therebetween.

In another embodiment of this aspect and all other aspects provided herein, the penetration depth is at least ⅓ (33%) the depth of the tumor. In other embodiments the penetration depth is at least 40% of the depth of the tumor, at least 50%, at least 60%, at least 70%, at least 75%, at least 80%, at least 90%, at least 95%, at least 99% the depth of the tumor. In some embodiments, the penetration depth does not need to penetrate the tumor tissue itself, provided that the fractional laser treatment induces a localized immune response within the tumor or along the borders of the tumor.

In some embodiments, a fractional laser comprises at least one shielding member configured to mask at least one portion of a target area of e.g., a tumor from electromagnetic radiation, in which the shielding members are formed such that a minimal amount of electromagnetic radiation is reflected back towards an electromagnetic radiation source. The regions where the energy passes through such a shielding device and into the tissue are the “microtreatment zones.”

In some embodiments, a fractional laser device can comprise a delivery module and translator. The delivery module is configured to direct electromagnetic radiation generated by an electromagnetic radiation source to a predetermined area within a target region of e.g., a tumor, wherein the predetermined area is located in a location relative to the delivery module, and wherein the electromagnetic radiation is adapted to cause thermal damage to tissue of the predetermined area or tumor region within. The translator is capable of moving the delivery module, such that the delivery module targets a plurality of spatially separated individual exposure areas of the predetermined area.

The delivery of the electromagnetic radiation to the target treatment area or tumor in a predetermined pattern is achieved by either masking parts of the target area or tumor in order to protect the masked parts of the tissue from the electromagnetic radiation, or by utilizing a light beam of relatively small diameter which is scanned across the surface of the targeted area (e.g. tumor) by various means in order to generate a specific pattern for affecting superficial thermal injury.

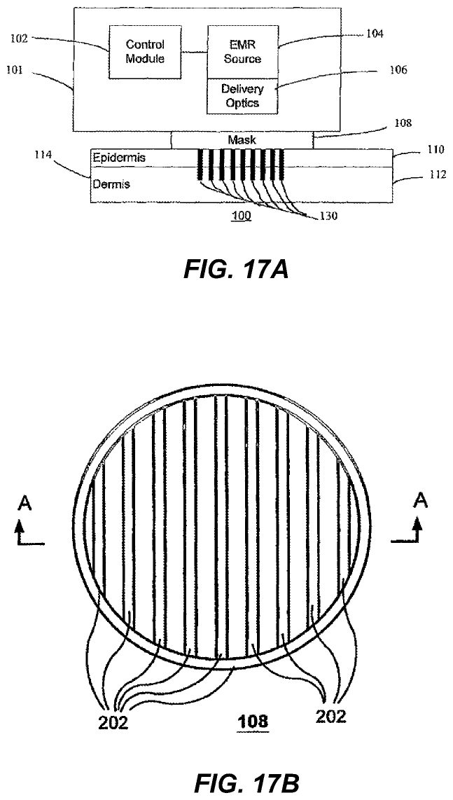

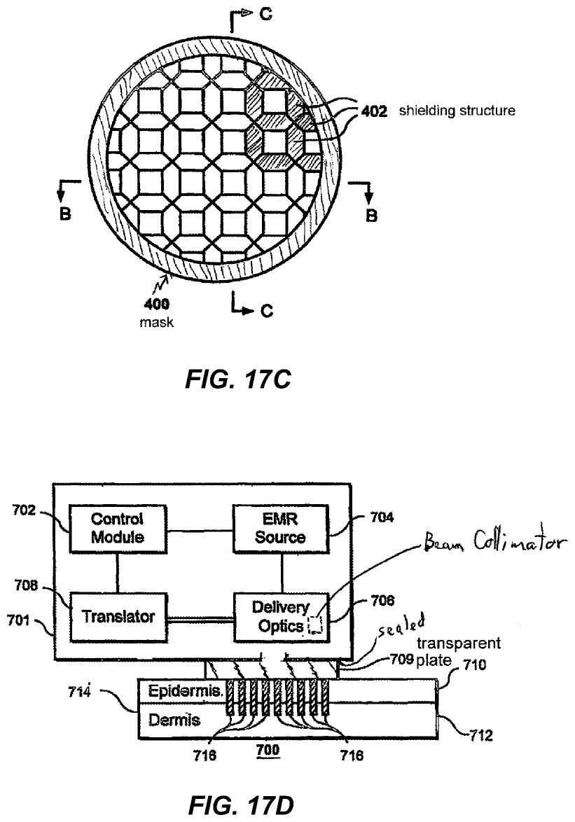

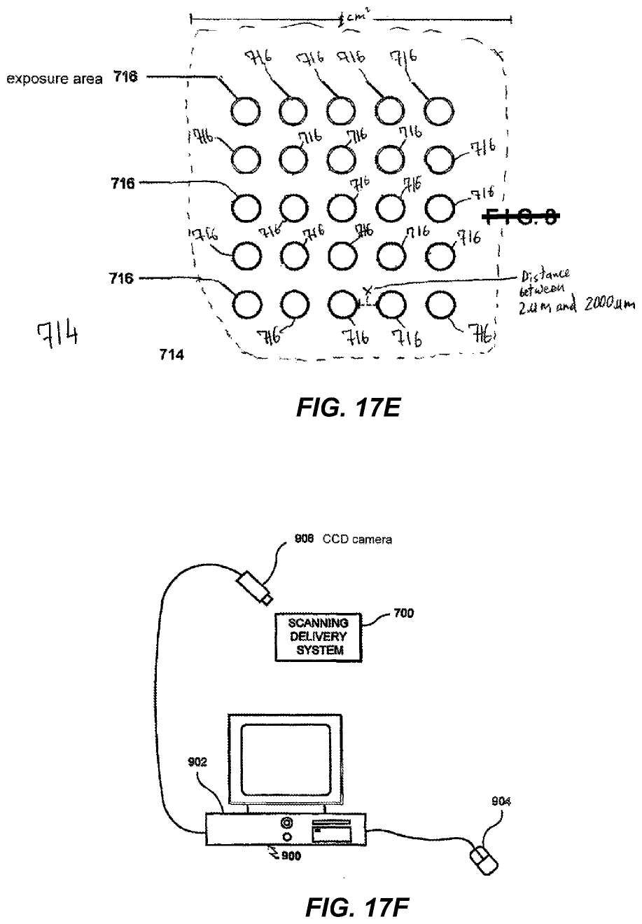

Exemplary, Fractional Laser Devices: An exemplary fractional laser device is shown herein in FIG. 17, which illustrates a fractional damage system 100 for conducting various treatments using electromagnetic radiation (“EMR”) and generating a pattern of microtreatment zones within a target area (e.g., a tumor) by using a mask or shielding device. As shown in FIG. 17 the system 100 includes a case 101, a control module 102, an EMR source 104, delivery optics 106 and a mask or shielding device 108. The case 101 contains the control module 102, the EMR source 104, and the delivery optics 106. An aperture is provided through a sidewall of the case 101. The mask 108 is placed in registration with the aperture formed through the skywall of the case 101. By placing, the mask 108 in registration with the aperture of the case 101, the focal length of the EMR emitted by the delivery optics 106 is fixed, and can be configured such that it does not impact the side of the mask 108, so as to cause injuries to the operator of the fractional damage system 100. The control module 102 is in communication with the EMR source 104, which in turn is operatively connected to the delivery optics 106.

In one embodiment, the fractional laser device can comprise the control module 102, which can be in wireless communication with the EMR source 104. In another embodiment, the control module 102 can be in wired communication with the EMR source 104. In another embodiment, the control module 102 can be located outside of the case 101. In another variant, the EMR source 104 is located outside of the case 101. In still another variant, the control module 102 and the EMR source 104 are located outside of the case 101. It is also possible that the mask 108 is not connected to the case 101.

The control module 102 provides application specific settings to the EMR source 104, The EMR source 104 receives these settings, and generates EMR based on these settings. The settings can control the wavelength of the EMR, the energy delivered to the tumor or lesion, the power delivered to the target area, the pulse duration for each EMR pulse, the fluence of the EMR delivered, the number of EMR pulses, the delay between individual EMR pulses, the beam profile of the EMR, and the size of the area within the mask exposed to EMR. The energy produced by the EMR source 104 can be an optical radiation, which is focused, collimated and/or directed by the delivery optics 106 to the mask 108. The mask 108 can be placed on a target area of a patient's tumor or lesion site, and can provide a damage pattern on the target area with a fill factor in the range from 0.1% to 24.9%. The fill factor is the percentage of the target area exposed to the EMR that is emitted by the EMR source 106.

In one exemplary embodiment, the EMR source 106 is one of a laser, a flashlamp, a tungsten lamp, a diode, a diode array, and the like. In another exemplary embodiment, the EMR source 106 is one of a CO₂ laser and a Er:YAG laser.

Prior to being used to generate fractional tissue damage, the system 100 shown in FIG. 17 can be configured by a user. For example, the user may interface with the control module 102 in order to specify the specific settings usable for a particular procedure. The user can specify the wavelength of the EMR, the energy delivered to the tumor, the power delivered to the tumor, the pulse duration for each EMR pulse, the fluence of the EMR delivered to the tumor, the number of EMR pulses, the delay between individual EMR pulses, the beam profile of the EMR, and the size of the area within the mask exposed to EMR. The EMR source 104 can be set to produce a collimated pulsed EMR irradiation with a wavelength ranging from 400 to 11,000 nm, and preferably near 3.0 μm when using an Er:YAG laser and near 10.6 μm when using a CO₂ laser as the EMR source. The collimated pulsed EMR irradiation can be applied which has a pulse duration in the range of 1 μs to 10 s, preferably in the range of 100 μs to 100 ms, and more preferably in the range of 0.1 ms to 10 ms, and fluence in the range from 0.01 to 100 J/cm², and preferably in the range from 1 to 10 J/cm². The applied EMR should be able to achieve at least a temperature rise within the exposed areas that is sufficient to cause thermal damage to the target tumor site). The peak temperature sufficient to cause thermal damage in the exposed tissues is time dependent and at least in the range of 45° C. to 100° C. For exposure times in the range of 0.1 ms to 10 ms the minimum temperature rise required to cause thermal damage is in the range of approximately 60° C. to 1000° C. The depth of thermal damage can be adjusted by proper choice of wavelength, fluence per pulse and number of pulses.

During the treatment to induce fractional damage, the system 100 produces EMR 120 which is directed to the target area 114, as shown in FIG. 17. The EMR 120 may be pulsed multiple times to create the appropriate effect and irradiation in the target area 114.

After the treatment is completed, the target area 114 is damaged in specific places or microtreatment zones. The application of the EMR 120 creates a prearranged thermal damage 130 in e.g., an epidermal tissue 110 and the dermal tissue 112. It should be noted that, in some embodiments, the thermal damage 130 extends through the epidermal tissue 10 and into the dermal tissue 112 only to a predetermined depth. FIG. 17 provides an embodiment in which energy is delivered through the epidermis to the dermis of the skin. It should be understood that similar principles and approaches apply when delivering energy to a tumor, whether exposed at a surgical site or contacted with EMR via, e.g., laparoscopic or other local tumor delivery approaches. The mask 108 controls in a location where the thermal damage 130 is created. The thermal damage 130 generally accounts for only 0.1% to 24.9% of the surface area in the target area. A fill factor is defined as the ratio of surface area of the target area thermally damaged by EMR to surface area of the target area.

It should be noted that it is possible that the penetration depths of each of the microtreatment zones of the thermal damage 130 can be different from one another or the same as one another.

FIG. 17B illustrates a top view of a first exemplary embodiment of the mask 108 for use with the methods and systems described herein. The mask 108 includes shielding structured 202. The diameter of the mask 108 should preferably be matched to greater than the size of the diameter of the target area. The target area is defined as the area targeted by the collimated. EMR emitted by the EMR source 104, which can be in the range 1400 mm in diameter, preferably within the range of 5 to 20 mm. This diameter of most of the currently commercially available CO₂ and Er:YAG laser systems can match the diameter of the exposed area. The width of shielding structures 202 within the mask 108 should be in the range of 50 to 300 μm. The width of the apertures of the mask 108 that are formed by the shielding structures should be in the range of 10-1000 μm, and preferably in the range of 50 to 300 μm. The shielding-exposure ratio surface area covered by the of shielding structures 202 to the surface area exposed by the apertures will determine the fractional tissue damage that is induced. This also determines the fill factor and the pattern of the thermal damage of the tumor or target region. The depth of thermal damage is determined by the number of pulses, the fluence of the EMR and the wavelength of the EMR. The shielding-exposure ratio of the mask 108 will vary for different treatments, particular patient needs, particular patient indications, skin types and body areas.

The mask 108 can have a large shielding-exposure ratio at the edge of the mask 108 to generate a transition zone at the edge of resurfaced area. In another preferred embodiment, a mask can be used that has a large shielding-exposure ratio at the edge of a conventionally resurfaced area to generate a transition zone.

The surface of the mask 108 should preferably have a minimal absorption at the wavelength generated by the EMR source 104 for the particular treatment process. Such absorption can decrease the undesirable heating of the mask 108. The mask 108 can be coated by a metal material for effectuating a minimal absorption of the EMR. The design of the shielding structures 202 of the mask 108 generally takes into consideration safety aspects, including a back-reflected EMR in order to avoid EMR inflicted accidents. The shielding structures 202 are shaped in a peaked manner to minimize the amount of back reflected EMR. Also, with the mask 108 being connected to the case 101 the distance between the deliver optics 106 and the mask 108 is fixed, thereby minimizing the chances that EMR would be reflected back towards the user by hitting the edge of the mask 108. Additionally, the microstructure of the mask 108 can have a periodicity preferably in the range of the wavelength of the EMR emitted by the delivery optics 106. This configuration can diffuse the collimated EMR emitted by the delivery optics 106 into a highly scattered beam so as to decrease the risk of EMR-related accidents.

In one exemplary embodiment, the metal coating of the mask 108 can be composed of gold, silver, or copper materials, or the like. In another exemplary embodiment, the microstructure of the surface of the mask 108 can have a periodicity in the range of the wavelength of the EMR emitted by the delivery optics 106.

The mask 108 can also have a configuration so as to provide effective surface cooling during the exposure thereof with the EMR radiation. Cooling can provide, for example, anesthetic effects, and has other advantages related to the pattern induced by the EMR radiation. The mask 108 can be cooled prior to the beginning of the fractional damage procedure, during the procedure by spraying an evaporative agent or a precooled liquid onto the mask 108 between the successive EMR pulses, or during the procedure by introducing a cool or cold liquid into microchannels running through the mask 108. The cooling of the mash 108 has a secondary advantage in that such cooling of the mask 108 decreases the rate of the EMR absorption by the mask 108, as the rate of the EMR absorption by the metals increases with the increasing temperature.

In order to provide cooling as described above, the temperature of the mask 108 should be in the range of 37° C. to −20° C., and preferably 10° C. to −4° C. The mask 108 can both protect and cool the portions of the treated tissue surface that are not exposed to EMR emitted by the EMR source 104. In addition to cooling and shielding portions of the treated surface, the mask 108 allows the debris ejected during ablative procedures to escape, and thereby not interfere with the beam delivery for successive pulses. For example, the areas that are not exposed to the laser are being cooled by the mask 108, i.e., the areas that are provided between the affected areas. In another exemplary embodiment, all areas (i.e., both the affected and nonaffected areas) are cooled to provide anesthesia, and to reduce over-damaging the superficial levels of the damaged areas.

FIG. 17C illustrates atop view of a second embodiment of the mask 400 useful with the methods and systems described herein. The mask 400 differs from the mash 108 only in the layout and design of the shielding structures 402. The details of the mask 400 are in all other respects substantially similar to those of the mask 108.

The induction of fractional tissue damage can be augmented using a short pulsed EMR. In a short pulsed-laser application, the laser can be pulsed for short periods of time, preferably for less than 1 μs in duration. The EMR source used in this type of procedure can preferably be a Q-switched ruby laser, a Nd:YAG laser, a KTP laser and/or an Alexandrite laser.

As an alternative to the fractional tissue damage using a mask, a second embodiment of a fractional resurfacing system 700, as shown as the progressive use thereof in FIG. 17D can be used. The system 700 can include a case 701, a control module 702, an electromagnetic radiation (“EMR”) source 704, delivery optics 706, an x-y translator 708 and an optically transparent plate 709. The case 701 can contain the control module 702, the EMR source. 704, the delivery optics 706 and the translator 708. As with the system 100, an aperture can be formed through a sidewall of the case 701. The optically transparent plate 709 can be placed in registration with the aperture that is formed through the sidewall of the case 701. Placing the plate 709 in registration with the aperture formed through the sidewall of the ease 701 seals the system 700, which contains sophisticated translation mechanisms, e.g., the delivery optics 706 and the translator 708. The control module 702 is in communication with the translator 708 and the EMR source 704 and the EMR source 704 is operatively connected to the delivery optics 706.

In one exemplary embodiment, the control module 702 can be located outside of the case 701. In another embodiment, the EMR source 704 is located outside of the case 701. In still another variant, the control module 702 and the EMR source 704 are located outside of the case 701.

The control module 702 provides application specific settings to the EMR source 704, and controls the x-y translator 708, The EMR source 704 receives these settings, and generates EMR based on these settings. The settings can control the wavelength of the energy produced, the intensity of the energy produced, the fluence of the energy produced, the duration of the procedure, the pulse length of each of the EMR pulses administered during the procedure, the spatial distance between individual exposure areas 716 (shown in 17E), the shape of individual exposure areas 716, the pattern defined by individual exposure areas 716, and the fill factor of the target area. It should be noted that the thermal damage caused to individual exposure areas 716 extends only to a predetermined depth. The EMR source 704 can be a laser or other light source. The EMR produced by the EMR source 704 can be delivered through a fiber, waveguide or mirrors if the source is located outside the delivery optics 706. Alternatively, if the EMR source 704 is located in a close vicinity to the skin 714, the EMR source 704 produces the EMR directly to the delivery optics 706. The energy produced by the EMR source 704 can be focused and/or directed by focusing optics in the delivery optics 706 to one of the individual exposure areas 716, shown in FIG. 17E. Each of the individual exposure areas 716 are located within the target area 714, and are relatively small compared to the target area of the tumor 714. The target area 714 can generally be, for example, 1 cm² in size and each of the individual exposure areas 716 may be 100 μm in diameter.

In an exemplary embodiment, the optics of the delivery optics 706 can contain a beam collimator focusing optics.

Prior to use in a method for generating fractional tissue damage and similarly to the use of system 100, the system 700, as shown in FIG. 17D, can be configured by a user. In particular, the user interfaces with the control module 702 in order to specify the specific settings to be used for a particular procedure. The user can specify the desired damage pattern, the wavelength of the energy produced by the EMR source 704, the intensity of the energy produced, the fluence of the energy produced, the length of time the treatment will take and the pulse duration of the EMR source 704. During the treatment, the translator 708 moves the delivery optics 706 across sequential portions of the target area 714 in order to treat the entire target area. The target area is treated when the system 700 delivers EMR to individual exposure areas 716 of the target area. The individual exposure areas 716 can be targeted serially and/or in parallel. When one of the micro-treatment zones within the target area has been completely treated, the system 700 is moved to the next portion of the target area. For example, the system 700 is moved at the completion of irradiation of each microtreatment zone within the target area until the desired fractional damage pattern is achieved for the entire area. The system 700 can be moved using discrete movements from one sequential portion to the next, i.e., stamping mode, or using continuous movement across the tissue surface, i.e., continuous scanning mode. In either case, the movement of the delivery optics 706, driven by the translator 708, is controlled by the control unit 702 and likely matched with the movement of the system 700 by the operator (or the user) in order to provide, the desired surface damage pattern to the target area (e.g., tumor) 714.

In an exemplary embodiment, the system 700, while operating in the continuous scanning mode, can deliver EMR to a particular individual exposure area 716, then, after exposure of such area 716, translate along the surface of the target area, and thereafter deliver a further EMR to another individual exposure area 716 separated from the previous particular individual exposure area 716 by a non-irradiated region. In another exemplary embodiment, the system 700, while operating in the continuous scanning mode, can deliver EMR to a particular group of individual exposure areas (e.g. microtreatment zones) 716, for example the top row of individual exposure areas 716 (shown in FIG. 17E), then, after exposure of such areas 716, translate, along the target area, and deliver a further EMR to another group of individual exposure areas 716, for example the second row of individual exposure areas 716 (shown in FIG. 17E), separated from the particular group of individual exposure areas 716 by non-irradiated areas.

In an exemplary embodiment, the system 700 includes a position sensor, which is in communication with the control module 702. The position sensor is capable of sensing the relative velocity as between the target area 114 and the case 701. The position sensor can be an optical mouse, wheels, track ball, conventional mouse, and the like.

In another exemplary embodiment, the system 700 targets individual exposure areas 716 one at

In a further exemplary embodiment, the system 700 creates individual exposure areas 716 having a separation distance between each of the individual exposure areas 716 of approximately at least 125 μm and at most 500 μm, preferably, the separation distance is approximately at least 250 μm.

Before the initiation of a procedure to induce fractional tissue damage, an optically transparent plate 709 can be brought into direct contact with the target area. The optically transparent plate 709 can be composed out of any material having good thermal conductivity, and being transparent over a broad range of the visible and near infrared spectrum. The plate 709 seals the system 700, which contains sophisticated translation mechanisms, and provides cooling to the target area 714. The plate 709 can provide cooling to the target area 714 in two ways: heat conduction and heat convection. Heat conduction transfers heat through the optically transparent plate. 709 to the case 701, which provides cooling by circulating a coolant agent through the case 701 of the system 700. The entire optically transparent plate 709 can also be cooled prior to application to the target area 714. Alternatively, heat convection can be utilized for this procedure. An evaporating agent sprayed onto the optical window or onto a compartment in good thermal contact with the window may also be utilized. The delivery of the evaporating agent can be administered during the procedure between EMR pulses through a valve, which can be controlled by a thermostat with a temperature sensor at the optical plate.

In one embodiment, the optically transparent plate 709 can be composed of sapphire or quartz. In another embodiment, the system 700 can be moved multiple times over the same target area 714 until the desired fill factor is achieved.

During a fractional tissue damage procedure, the EMR source 704 emits EMR having a wavelength in the range of 400-12,000 nm. Preferably the EMR has a wavelength in one of the following ranges: 1,300 to 1,600 nm, 1,850 to 2,100 nm, 2,300 to 3,100 nm and around 10,640 nm. Depending on the application, a single wavelength or a combination of different wavelengths can be utilized. The EMR source 704 can be a diode laser, a fiber laser, a solid state laser, a gas laser, and the like. The pulse duration can range from 100 μs to 100 ms, and preferably in the range from 500 μs to 15 ms, and more preferably in the range from 1.5 ms to 5 ms. The energy density per pulse within an individual exposure area 716 can be in the range of 0.1 to 100 J/cm², preferably 1 to 32 J/cm², and more preferably 1.5 to 3 J/cm². The energy per pulse within an individual exposure area 716 can be in the range of 1 mJ and 10 mJ, and preferably 5 mJ.

In an exemplary embodiment, the EMR source 704 is a 1.5 μm laser system, such as a Reliant FSR prototype, manufactured by Reliant Technologies, Palo Alto, Calif., is used.

FIG. 17E illustrates a top view of the small individual exposure areas 716 of a target area tumor). The shape of the individual exposure areas 716 can be circular, elliptical, rectangular, linear or irregular with a lateral diameter of the smallest dimension in the range of 1-500 μm. The fill factor of the target area can be approximately 20-40%.

FIG. 17F illustrates an exemplary embodiment of a monitoring system 900 for use with the systems described herein. The monitoring system 900 tracks the movement of the system 700, and feeds such positional information to the control module 702. The control module 702 utilizes this information to appropriately instruct the translator 708 to position the delivery optics 706, such that the appropriate damage pattern is achieved across the target area 714. The monitoring system 900 can use a computer 902, a mouse 904, and a charge coupled device (“CCD”) camera 906. In particular, the computer 902 receives the positional information about the system 700 from the CCD camera 906. The computer then updates the control module 702 based on this positional information as to the current position of the system 700. The control module 702 utilizes this information to cause the system 700 to create the appropriate damage pattern 714 within the target area. In addition, the monitoring system can utilize additional motion detecting devices, including, wheels or any other motion sensor.

The shape of the individual exposure areas 716 and the relative pattern represented by all of the individual exposure areas 716 can vary. The individual exposure areas 716 can have a circular, elliptical, rectangular, linear or irregular shape. The average distance between individual regions of exposed surface can be in the range between 10 to 2000 μm, and preferably in the range of 100 to 500 μm. The macroscopic pattern of the individual exposure areas 716 can be a field of uniformly distributed individual exposure areas 716 with constant spacing throughout the target area, randomly distributed individual exposure areas 716 within the target area, and/or regularly distributed individual exposure areas 716 with constant average spacing with randomly shifted location. However, uniformly distributed individual exposure areas 716 with constant spacing throughout the target area may create unwanted spatial distributions similar to moire patterns, resulting in spatial interference macroscopic patterns generated with a distance in between the areas of exposure which have a significant spatial period. In order to minimize the occurrence of moire patterns, a randomized shift within the range of 10 to 50% of the average distance between individual exposure areas 716 during a single scan may be utilized.

### Immune Checkpoint Molecules and Immune Checkpoint Modulators Thereof

The immune system has multiple inhibitory pathways that are critical for maintaining self-tolerance and modulating immune responses. In T-cells, the amplitude and quality of response is initiated through antigen recognition by the T-cell receptor and is regulated by immune checkpoint proteins that balance co-stimulatory and inhibitory signals. In some embodiments, the immune checkpoint modulator is an inhibitor of a blocking checkpoint molecule. Exemplary blocking checkpoint molecules include, but are not limited to, PD-1, PD-L1, CTLA4, B7-H3, B7-H4, VISTA, TMIGD2, B7-H7, BTLA, HVEM, CD160, LAG3, TIGIT, CD96, CD155, TIM-3, Galectin-9, Adenosine, Adenosine A2a receptor, IDO, TDO, CEACAM1, SIRP alpha, CD47, CD200R and CD200. In other embodiments, the immune checkpoint modulator is an agonist of a stimulative checkpoint molecule. Exemplary stimulative checkpoint molecules include, but are not limited to, OX40, 4-1BB, GITR, CD28, ICOS, LIGHT, CD27, DNAM-1, 2B4, DC-SIGN, DR3, and CD40.

Further examples of checkpoint molecules that can be targeted for blocking or inhibition include, but are not limited to, PDL2, B7-H3, B7-H4, BTLA, HVEM, GALS, VISTA, KIR, 2B4 (belongs to the CD2 family of molecules and is expressed on all NK, γδ, and memory CD8+(αβ) T cells), CD160 (also referred to as BY55), CGEN-15049, CHK 1 and CHK2 kinases, A2aR, TIGIT, DD1-α, TIM-3, Lag-3, and various B-7 family ligands. B7 family ligands include, but are not limited to, B7-1, B7-2, B7-DC, B7-H1, B7-H2, B7-H3, B7-H4, B7-H5, B7-H6 and B7-H7.

Immune checkpoints and modulators thereof as well as methods of using such compounds are known to those of skill in the art and/or are described in the literature, thus they are not described in detail herein. A brief description of immune checkpoint molecules and modulators thereof is provided below.

In some embodiments, the one or more immune checkpoint modulator(s) may independently be a polypeptide or a polypeptide-encoding nucleic acid molecule; said polypeptide comprising a domain capable of binding the desired immune checkpoint molecule and/or inhibiting the binding of a ligand to the immune checkpoint molecule so as to exert an antagonist function (i.e. being capable of antagonizing an immune checkpoint-mediated inhibitory signal) or an agonist function (i.e. being capable of boosting an immune checkpoint-mediated stimulatory signal). Such one or more immune checkpoint modulator(s) can be independently selected from the group consisting of peptides (e.g. peptide ligands), soluble domains of natural receptors, RNAi, antisense molecules, antibodies and protein scaffolds.

In certain embodiments, the immune checkpoint modulator is an antibody. The term “antibody” (“Ab”) is used in the broadest sense and encompasses those naturally occurring and engineered by man as well as full length antibodies or functional fragments (e.g., an scFv) or analogs thereof that are capable of binding the target immune checkpoint molecule or epitope thereof (thus retaining the target-binding portion). Such antibodies can be of any origin, e.g. human, humanized, animal (e.g. rodent or camelid antibody) or chimeric. It may be of any isotype with a specific preference for an IgG1 or IgG4 isotype. In addition, it may be glycosylated or non-glycosylated. The term antibody also includes bispecific or multispecific antibodies so long as they exhibit the binding specificity described herein.

As a brief description for illustrative purposes, full length antibodies are glycoproteins comprising at least two heavy (H) chains and two light (L) chains inter-connected by disulfide bonds. Each heavy chain is comprised of a heavy chain variable region (VH) and a heavy chain constant region which is made of three CH1, CH2 and CH3 domains (eventually with a hinge between CH1 and CH2). Each light chain is comprised of a light chain variable region (VL) and a light chain constant region which comprises one CL domain. The VH and VL regions comprise hypervariable regions, named complementarity determining regions (CDR), interspersed with more conserved regions named framework regions (FR). Each VH and VL is composed of three CDRs and four FRs in the following order: FR1-CDR1-FR2-CDR2-FR3-CDR3-FR4. The CDR regions of the heavy and light chains are determinant for the binding specificity.

As used herein, the term “humanized antibody” refers to a non-human (e.g. murine, camel, rat, etc) antibody whose protein sequence has been modified to increase its similarity to a human antibody (i.e. produced naturally in humans). The process of humanization is well known in the art (see e.g. Presta et al., 1997, Cancer Res. 57(20): 4593-9; U.S. Pat. Nos. 5,225,539; 5,530,101; 6,180,370; WO2012/110360). For example, a monoclonal antibody developed for human use can be humanized by substituting one or more residue of the FR regions to look like human immunoglobulin sequence whereas the vast majority of the residues of the variable regions (especially the CDRs) are not modified and correspond to those of a non-human immunoglobulin. For general guidance, the number of these amino acid substitutions in the FR regions is typically no more than 20 in each variable region VH or VL.

As used herein, a “chimeric antibody” refers to an antibody comprising one or more element(s) of one species and one or more element(s) of another species, for example, a non-human antibody comprising at least a portion of a constant region (Fc) of a human immunoglobulin.

Many forms of antibody can be engineered for use in the combination of the invention. Representative examples include without limitation Fab, Fab′, F(ab′)2, dAb, Fd, Fv, scFv, di-scFv and diabody, etc. More specifically:
(i) a Fab fragment represented by a monovalent fragment consisting of the VL, VH, CL and CH1 domains;
(ii) a F(ab′)2 fragment represented by a bivalent fragment comprising two Fab fragments linked by at least one disulfide bridge at the hinge region;
(iii) a Fd fragment consisting of the VH and CH1 domains;
(iv) a Fv fragment consisting of the VL and VH domains of a single arm of an antibody,
(v) a dAb fragment consisting of a single variable domain fragment (VH or VL domain);
(vi) a single chain Fv (scFv) comprising the two domains of a Fv fragment, VL and VH, that are fused together, eventually with a linker to make a single protein chain (see e.g. Bird et al., 1988, Science 242: 423-6; Huston et al., 1988, Proc. Natl. Acad. Sci. USA 85: 5879-83; U.S. Pat. Nos. 4,946,778; 5,258,498); and
(vii) any other artificial antibody.

Methods for preparing antibodies, fragments and analogs thereof are known in the art (see e.g. Harlow and Lane, 1988, Antibodies—A laboratory manual; Cold Spring Harbor Laboratory, Cold Spring Harbor N.Y.). One may cite for example hybridoma technology (as described in Kohler and Milstein, 1975, Nature 256: 495-7; Cote et al., 1983, Proc. Natl. Acad. Sci. USA 80: 2026-30; Cole et al. in Monoclonal antibodies and Cancer Therapy; Alan Liss pp 77-96), recombinant techniques (e.g. using phage display methods), peptide synthesis and enzymatic cleavage. Antibody fragments can be produced by recombinant technique as described herein. They can also be produced by proteolytic cleavage with enzymes such as papain to produce Fab fragments or pepsin to produce F(ab′)2 fragments as described in the literature (see e.g. Wahl et al., 1983, J. Nucl. Med. 24: 316-25). Analogs (or fragment thereof) can be generated by conventional molecular biology methods (PCR, mutagenesis techniques). If needed, such fragments and analogs may be screened for functionality in the same manner as with intact antibodies (e.g. by standard ELISA assay).

In some embodiments, at least one of the immune checkpoint modulator(s) for use with the methods and systems described herein is a monoclonal antibody, with a specific preference for a human (in which both the framework regions are derived from human germline immunoglobin sequences) or a humanized antibody according to well-known humanization process.

Desirably, the one or more immune checkpoint modulator(s) in use in the methods and compositions described herein antagonizes at least partially (e.g. more than 50%) the activity of inhibitory immune checkpoint(s), in particular those mediated by any of the following non-limiting examples: PD-1, PD-L1, PD-L2, LAG3, Tim3, KIR, BTLA and CTLA4, with a specific preference for a monoclonal antibody that specifically binds to any of such target proteins. The term “specifically binds to” refers to the capacity to a binding specificity and affinity for a particular target or epitope even in the presence of a heterogeneous population of other proteins and biologics. Thus, under designated assay conditions, the antibody binds preferentially to its target and does not bind in a significant amount to other components present in a test sample or subject. Preferably, such an antibody shows high affinity binding to its target with an equilibrium dissociation constant equal to or below 1×10⁻⁶M (e.g. at least 0.5×10⁻⁶, 1×10⁻⁷, 1×10⁻⁸, 1×10⁻⁹, 1×10⁻¹⁰, etc). Alternatively, an immune checkpoint modulator(s) in use with the methods described herein can exert an agonist function in the sense that it is capable of stimulating or reinforcing stimulatory signals, in particular those mediated by CD28 with a specific preference for any of e.g., ICOS, CD137 (or 4-1BB), OX40, CD27, CD40 and GITR immune checkpoint molecules. Standard assays to evaluate the binding ability of the antibodies toward immune checkpoints are known in the art, including for example, ELISAs, Western blots, RIAs and flow cytometry. The binding kinetics (e.g., binding affinity) of the antibodies also can be assessed by standard assays known in the art, such as by Biacore analysis.

In certain embodiments, at least one of the immune checkpoint modulator(s) for use as described herein comprises a human or a humanized antibody capable of antagonizing an immune checkpoint involved in a T cell-mediated response.

In one embodiment, the blocking checkpoint molecule or stimulative checkpoint molecule modulates the activity of the programmed cell death 1 (PD-1)/programmed cell death ligand (PD-L1) signaling pathway. A non-limiting example of an immune checkpoint modulator is represented by a modulator capable of antagonizing at least partially the protein Programmed Death 1 (PD-1), and especially an antibody that specifically binds to human PD-1. PD-1 is part of the immunoglobulin (Ig) gene superfamily and a member of the CD28 family. It is a 55 kDa type 1 transmembrane protein expressed on antigen-experienced cells (e.g. activated B cells, T cells, and myeloid cells) (Agata et al., 1996, Int. Immunol. 8: 765-72; Okazaki et al., 2002, Curr. Opin. Immunol. 14: 391779-82; Bennett et al., 2003, J. Immunol 170: 711-8). In normal context, it acts by limiting the activity of T cells at the time of inflammatory response, thereby protecting normal tissues from destruction (Topalian, 2012, Curr. Opin. Immunol. 24: 207-12). Two ligands have been identified for PD-1, respectively PD-L1 (programmed death ligand 1) and PD-L2 (programmed death ligand 2) (Freeman et al., 2000, J. Exp. Med. 192: 1027-34; Carter et al., 2002, Eur. J. Immunol. 32: 634-43). PD-L1 was identified in 20-50% of human cancers (Dong et al., 2002, Nat. Med. 8: 787-9). The interaction between PD-1 and PD-L1 resulted in a decrease in tumor infiltrating lymphocytes, a decrease in T-cell receptor mediated proliferation, and immune evasion by the cancerous cells (Dong et al., 2003, J. Mol. Med. 81: 281-7; Blank et al., 2005, Cancer Immunol. Immunother. 54: 307-314). The complete nucleotide and amino acid PD-1 sequences can be found under GenBank Accession No U64863 and NP 005009.2. A number of anti PD1 antibodies are available in the art (see e.g. those described in WO2004/004771; WO2004/056875; WO2006/121168; WO2008/156712; WO2009/014708; WO2009/114335; WO2013/043569; and WO2014/047350, the contents of each of which are incorporated herein by reference in their entirety). Preferred anti PD-1 antibodies for use with the methods described herein are FDA approved or under advanced clinical development and one may use in particular an anti-PD-1 antibody selected from the group consisting of Nivolumab (also termed BMS-936558 under development by Bristol Myer Squibb), Pembrolizumab (also termed Lanbrolizumab or MK-3475; under development by Merck), and Pidilizumab (also termed CT-011 under development by CureTech).

Another non-limiting example of an immune checkpoint modulator is represented by a modulator capable of antagonizing, at least partially, the PD-1 ligand termed PD-L1, and especially an antibody that recognizes human PD-L1. A number of anti PD-L1 antibodies are available in the art (see e.g. those described in EP1907000). Preferred anti PD-L1 antibodies are FDA approved or under advanced clinical development (e.g. MPDL3280A under development by Genentech/Roche and BMS-936559 under development by Bristol Myer Squibb).

Cytotoxic T-lymphocyte associated antigen 4 (CTLA-4) is an immune checkpoint protein that down-regulates pathways of T-cell activation (Fong et al., Cancer Res. 69(2):609-615, 2009; Weber Cancer Immunol. Immunother, 58:823-830, 2009). CTLA4 (for cytotoxic T-lymphocyte-associated antigen 4) also known as CD152 was identified in 1987 (Brunet et al., 1987, Nature 328: 267-70) and is encoded by the CTLA4 gene (Dariavach et al., Eur. J. Immunol. 18: 1901-5). CTLA4 is a member of the immunoglobulin superfamily of receptors. It is expressed on the surface of helper T cells where it primarily regulates the amplitude of the early stages of T cell activation. Recent work has suggested that CTLA-4 may function in vivo by capturing and removing B7-1 and B7-2 from the membranes of antigen-presenting cells, thus making these unavailable for triggering of CD28 (Qureshi et al., Science, 2011, 332: 600-3). The complete CTLA-4 nucleic acid sequence can be found under GenBank Accession No LI 5006. Blockade of CTLA-4 has been shown to augment T-cell activation and proliferation. Inhibitors of CTLA-4 include anti-CTLA-4 antibodies. Anti-CTLA-4 antibodies bind to CTLA-4 and block the interaction of CTLA-4 with its ligands CD80/CD86 expressed on antigen presenting cells, thereby blocking the negative down regulation of the immune responses elicited by the interaction of these molecules. Examples of anti-CTLA-4 antibodies are described in U.S. Pat. Nos. 5,811,097; 5,811,097; 5,855,887; 6,051,227; 6,207,157; 6,682,736; 6,984,720; and 7,605,238, the contents of each of which are incorporated herein by reference in their entirety. A non-limiting example of an anti-CDLA-4 antibody is tremelimumab, (ticilimumab, CP-675,206). In one embodiment, the anti-CTLA-4 antibody is ipilimumab (also known as 10D1, MDX-D010) a fully human monoclonal IgG antibody that binds to CTLA-4. Ipilimumab is marketed under the name Yervoy™ and has been approved for the treatment of unresectable or metastatic melanoma.

Additional anti-CTLA4 antagonists include, but are not limited to, the following: any inhibitor that is capable of disrupting the ability of CD28 antigen to bind to its cognate ligand, to inhibit the ability of CTLA4 to bind to its cognate ligand, to augment T cell responses via the co-stimulatory pathway, to disrupt the ability of B7 to bind to CD28 and/or CTLA4, to disrupt the ability of B7 to activate the co-stimulatory pathway, to disrupt the ability of CD80 to bind to CD28 and/or CTLA4, to disrupt the ability of CD80 to activate the co-stimulatory pathway, to disrupt the ability of CD86 to bind to CD28 and/or CTLA4, to disrupt the ability of CD86 to activate the co-stimulatory pathway, and to disrupt the co-stimulatory pathway, in general from being activated. This necessarily includes small molecule inhibitors of CD28, CD80, CD86, CTLA4, among other members of the co-stimulatory pathway; antibodies directed to CD28, CD80, CD86, CTLA4, among other members of the co-stimulatory pathway; antisense molecules directed against CD28, CD80, CD86, CTLA4, among other members of the co-stimulatory pathway; adnectins directed against CD28, CD80, CD86, CTLA4, among other members of the co-stimulatory pathway, RNAi inhibitors (both single and double stranded) of CD28, CD80, CD86, CTLA4, among other members of the co-stimulatory pathway, among other anti-CTLA4 antagonists.

In other embodiments, the CTLA4 inhibitor comprises an antibody that recognizes human CTLA-4. A number of anti CTLA-4 antibodies are available in the art (see e.g. those described in U.S. Pat. No. 8,491,895). Preferred anti CTLA-4 antibodies for use with the methods and systems described herein are FDA approved or under advanced clinical development. For example, a CTLA-4 antibody for use as described herein include ipilimumab marketed by Bristol Myer Squibb as Yervoy (see e.g. U.S. Pat. Nos. 6,984,720; 8,017,114), tremelimumab under development by—Pfizer (see e.g. U.S. Pat. Nos. 7,109,003 and 8,143,379) and single chain anti-CTLA4 antibodies (see e.g. WO97/20574 and WO2007/123737). The contents of each of the aforementioned patents and patent applications are incorporated by reference herein in their entirety.

Another non-limiting example of a checkpoint molecule is TIGIT (T cell Immunoreceptor with Ig and ITIM domains), also called WUCAM, VSIG9 or Vstm3, which is a co-inhibitory receptor preferentially expressed on NK, CD8+ and CD4+ T cells as well as on regulatory T cells (Treg cells, or simply “Tregs”). TIGIT is a transmembrane protein containing a known ITIM domain in its intracellular portion, a transmembrane domain and an immunoglobulin variable domain on the extracellular part of the receptor. Several ligands have been described to bind to the TIGIT receptor with CD155/PVR showing the best affinity followed by CD113/PVRL3 and CD112/PVRL2 (Yu et al. (2009) Nat. Immunol. 10:48.). DNAM/CD226, a known co-stimulatory receptor also expressed on NK and T cells competes with TIGIT for CD155 and CD112 binding but with a lower affinity, which suggests a tight control of the activation of these effector cells to avoid uncontrolled cytotoxicity against normal cells expressing CD155 ligand. TIGIT expression is increased on tumor infiltrating lymphocytes (TILs) and in disease settings such as HIV infection. TIGIT expression marks exhausted T cells that have lower effector function as compared to TIGIT negative counterparts (Kurtulus et al. (2015) J. Clin. Invest. 276:112; Chew et al. (2016) Plos Pathogens. 12). Conversely, Treg cells that express TIGIT show enhanced immunosuppressive activity as compared to TIGIT negative Treg population (Joller et al. (2014) Immunity. 40:569). Exemplary TIGIT antibodies are known in the art and/or are described in e.g., U.S. Pat. Nos. 10,329,349; 10,537,633; 10,047,158; 11,021,537; 11,008,390, the contents of each of which are incorporated herein by reference in their entirety.

Another exemplary immune checkpoint molecule comprises V-region Immunoglobulin-containing Suppressor of T cell Activation (VISTA) or PD-L3, which is a hematopoietically-restricted, structurally-distinct, Ig-superfamily inhibitory ligand designated as. The extracellular domain bears homology to the B7 family ligand PD-L1, and like PD-L1, VISTA has a profound impact on immunity. However, unlike PD-L1, expression of VISTA is exclusively within the hematopoietic compartment. Expression is most prominent on myeloid antigen-presenting cells (APCs), although expression on CD4+ T cells and on a subset of Foxp3+ regulatory T cells (Treg) is also of significant interest. Exemplary VISTA antibodies are described in U.S. Pat. Nos. 9,631,018; and 10,745,467, the contents of each of which are incorporated herein by reference in their entirety.

Other exemplary immune-checkpoint inhibitors include lymphocyte activation gene-3 (LAG-3) inhibitors, such as IMP321, a soluble Ig fusion protein (Brignone et al., 2007, J. Immunol. 179:4202-4211). Other immune-checkpoint inhibitors include B7 inhibitors, such as B7-H3 and B7-H4 inhibitors. In particular, the anti-B7-H3 antibody MGA271 (Loo et al., 2012, Clin. Cancer Res. July 15 (18) 3834). Also included are TIM3 (T-cell immunoglobulin domain and mucin domain 3) inhibitors (Fourcade et al., 2010, J. Exp. Med. 207:2175-86 and Sakuishi et al., 2010, J. Exp. Med. 207:2187-94).

Immune checkpoint modulators for antagonizing the LAG3 receptor can also be used in the methods described herein and are described in e.g., U.S. Pat. No. 5,773,578, the contents of which are incorporated herein by reference in their entirety

Another example of an immune checkpoint modulator is represented by an OX40 agonist such as agonist ligand of OX40 (Ox40L) (see e.g. U.S. Pat. Nos. 5,457,035, 7,622,444; WO03/082919, the contents of each of which are incorporated herein by reference in their entirety) or an antibody directed to the OX40 receptor (see e.g. U.S. Pat. No. 7,291,331 and WO03/106498, the contents of each of which are incorporated herein by reference in their entirety).

Another exemplary stimulative immune checkpoint molecule is Dendritic Cell-Specific Intercellular adhesion molecule-3-Grabbing Non-integrin (DC-SIGN), which is a C-type lectin receptor present on the surface of both macrophages and dendritic cells (Soilleux E J, et al. (2002) J Luekoc Biol. 71(3):445-57). DC-SIGN can initiate innate immunity by modulating toll-like receptors (den Dunnen J, et al. (2009) Cancer Immunol. Immunother. 58 (7): 1149-57). Exemplary DC-SIGN antibodies are described in e.g., US Patent Application No.: 2021/0170043, the contents of which are incorporated herein by reference in its entirety.

Other examples of immune checkpoint modulators are represented by anti-KIR or anti-CD96 antibody targeting the inhibitory receptors harbored by CD8+ T cells and NK cells.

The method described herein encompasses a combination comprising more than one immune checkpoint modulator. A non-limiting example includes using an OX40 agonist antibody and an anti-PD-1 antibody in combination with treatment to induce fractional damage in a tissue, lesion or tumor.

In some embodiments, an immune checkpoint modulator is selected from the group consisting of: anti-PD-1 antibodies (e.g., Nivolumab, Cemiplimab (REGN-2810), Pembrolizumab (MK-3475), Spartalizumab (PDR-001), Tislelizumab (BGB-A317), AMP-514 (MEDI0680), Dostarlimab (ANB011/TSR-042), Toripalimab (JS001), Camrelizumab (SHR-1210), Genolimzumab (CBT-501), Sintilimab (IBI308), STI-A1110, ENUM 388D4, ENUM 244C8, GLS010, MGA012, AGEN2034, CS1003, HLX10, BAT-1306, AK105, AK103, BI754091, LZM009, CMAB819, Sym021, GB226, SSI-361, JY034, HX008, ISU106, ABBV181, BCD-100, PF-06801591, CX-188 and JNJ-63723283, etc.), anti-PD-L1 antibodies (e.g., Atezolizumab (RG7446/MPDL3280A), Avelumab (PF-06834635/MSB0010718C), Durvalumab (MEDI4736), BMS-936559, STI-1014, KNO35, LY3300054, HLX20, SHR-1316, CS1001 (WBP3155), MSB2311, BGB-A333, KL-A167, CK-301, AK106, AK104, ZKAB001, FAZ053, CBT-502 (TQB2450), JS003 and CX-072, etc.), PD-1 antagonists (e.g., AUNP-12, the respective compounds such as BMS-M1 to BMS-M10 (see WO2014/151634, WO2016/039749, WO2016/057624, WO2016/077518, WO2016/100285, WO2016/100608, WO2016/126646, WO2016/149351, WO2017/151830 and WO2017/176608), BMS-1, BMS-2, BMS-3, BMS-8, BMS-37, BMS-200, BMS-202, BMS-230, BMS-242, BMS-1001, BMS-1166 (see WO2015/034820, WO2015/160641, WO2017/066227 and Oncotarget. 2017 Sep. 22; 8 (42): 72167-72181.), the respective compounds of Incyte-1 to Incyte-6 (see WO 2017/070089, WO2017/087777, WO2017/106634, WO 2017/112730, WO 2017/192961 and WO2017/205464), CAMC-1 to CAMC-4 (see WO 2017/202273, WO2017/202274, WO2017/202275 and WO2017/202276), RG_1 (see WO2017/118762) ant DPPA-1 (see Angew. Chem. Int. Ed. 2015, 54, 11760-11764), etc.), PD-L1/VISTA antagonists (e.g., CA-170 etc.), PD-L1/TIM3 antagonists (e.g., CA-327 etc.), anti-PD-L2 antibodies, PD-L1 fusion proteins, PD-L2 fusion proteins (e.g., AMP-224 etc.), anti-CTLA-4 antibodies (e.g., Ipilimumab (MDX-010), AGEN1884 and Tremelimumab, etc.), anti-LAG-3 antibodies (e.g., Relatlimab (BMS-986016/ONO-4482), LAG525, REGN3767 and MK-4280, etc.), LAG-3 fusion proteins (e.g., IMP321 etc.), anti-Tim3 antibodies (e.g., MBG453 and TSR-022, etc.), anti-KIR antibodies (e.g., Lililumab (BMS-986015, ONO-4483), IPH2101, LY3321367 and MK-4280, etc.), anti-BTLA antibodies, anti-TIGIT antibodies (e.g., Tiragolumab (MTIG-7192A/RG-6058/RO-7092284) and BMS-986207 (ONO-4686), anti-VISTA antibody (e.g., JNJ-61610588) and anti-CSF-1R antibody or CSF-1R inhibitor (e.g., Cabiralizumab (FPA008/BMS-986227/ONO-4687), Emactuzumab (RG7155/RO5509554), LY3022855, MCS-110, IMC-CS4, AMG820, Pexidartinib, BLZ945 and ARRY-382, etc.).

Additional immune checkpoint inhibitors and modulators thereof are known to those of skill in the art and are not described herein, however any immune checkpoint modulator can be used with the methods and systems described herein.

### Pharmaceutically Acceptable Carriers

Therapeutic compositions of the agents disclosed herein can include a physiologically tolerable carrier together with an immune checkpoint modulator as described herein, dissolved or dispersed therein as an active ingredient. As used herein, the terms “pharmaceutically acceptable”, “physiologically tolerable” and grammatical variations thereof, as they refer to compositions, carriers, diluents and reagents, are used interchangeably and represent that the materials are capable of administration to or upon a mammal without toxicity or the production of undesirable physiological effects such as nausea, dizziness, gastric upset and the like. A pharmaceutically acceptable carrier will not itself promote the raising of an immune response to an agent with which it is admixed, unless so desired. The preparation of a pharmacological composition that contains active ingredients dissolved or dispersed therein is well understood in the art and need not be limited based on formulation. Typically, such compositions are prepared as topical agents or injectable either as liquid solutions or suspensions, however, solid forms suitable for solution, or suspensions, in liquid prior to use can also be prepared. The preparation can also be emulsified or presented as a liposome composition. The active ingredient can be mixed with excipients which are pharmaceutically acceptable and compatible with the active ingredient and in amounts suitable for use in the therapeutic methods described herein.

### Dosage and Administration

In a treatment method as described herein, an effective amount of one more immune checkpoint modulators and/or fractional tissue damage that induces an immune response is administered to a patient suffering from or diagnosed as having a tumor (e.g., solid tumor or melanoma) or in need of an induced systemic immune response (e.g., having an infection or cancer). In one aspect, the methods described herein provide a method for treating cancer in a subject. In one embodiment, the subject can be a mammal (e.g., a primate or a non-primate mammal). In another embodiment, the mammal can be a human, although the approach is effective with respect to all mammals. An “effective amount” means an amount or dose generally sufficient to bring about the desired therapeutic or prophylactic benefit in subjects undergoing treatment.

Effective amounts or doses of one or more immune checkpoint modulators or fractional tissue damage for treatment as described herein can be ascertained by routine methods such as modeling, dose escalation studies or clinical trials, and by taking into consideration routine factors, e.g., the mode or route of administration of delivery, the pharmacokinetics of the composition, the severity and course of the disorder or condition, the subject's previous or ongoing therapy, the subject's health status and response to drugs, and the judgment of the treating physician. An exemplary dose for a human is in the range of from about 0.001 to about 8 mg per kg of subject's body weight per day, about 0.05 to 300 mg/day, or about 50 to 400 mg/day, in single or divided dosage units (e.g., BID, TID, QID).

While the dosage range for the one or more immune checkpoint modulators to induce the immune response depends upon the potency of the composition, and includes amounts large enough to produce the desired effect (e.g., improved tumor treatment), the dosage should not be so large as to cause unacceptable adverse side effects. Generally, the dosage will vary with the formulation (e.g., oral, i.v. or subcutaneous formulations), and with the age, condition, and sex of the patient. The dosage can be determined by one of skill in the art and can also be adjusted by the individual physician in the event of any complication. Typically, the dosage will range from 0.001 mg/day to 400 mg/day. In some embodiments, the dosage range is from 0.001 mg/day to 400 mg/day, from 0.001 mg/day to 300 mg/day, from 0.001 mg/day to 200 mg/day, from 0.001 mg/day to 100 mg/day, from 0.001 mg/day to 50 mg/day, from 0.001 mg/day to 25 mg/day, from 0.001 mg/day to 10 mg/day, from 0.001 mg/day to 5 mg/day, from 0.001 mg/day to 1 mg/day, from 0.001 mg/day to 0.1 mg/day, from 0.001 mg/day to 0.005 mg/day. Alternatively, the dose range will be titrated to maintain serum levels between 0.1 μg/mL and 30 μg/mL.

It is also contemplated herein that the dose of e.g., a checkpoint modulator to produce a desired effect can be reduced when administered in combination with e.g., ablative FP compared to the dose that is administered for conventional treatment of the cancer.

Administration of the doses recited above can be repeated for a limited period of time or as necessary. In some embodiments, the doses are given once a day, or multiple times a day, for example but not limited to three times a day. In one embodiment, the doses recited above are administered daily for several weeks or months. The duration of treatment depends upon the subject's clinical progress and responsiveness to therapy. Continuous, relatively low maintenance doses are contemplated after an initial higher therapeutic dose.

Agents useful in the methods and compositions described herein depend on the site of the tumor and can be administered topically, intravenously (by bolus or continuous infusion), intratumorally, orally, by inhalation, intraperitoneally, intramuscularly, subcutaneously, intracavity, and can be delivered by peristaltic means, if desired, or by other means known by those skilled in the art. For the treatment of certain cancers (e.g., metastatic disease), the agent can be administered systemically.

Therapeutic compositions containing at least one agent can be conventionally administered in a unit dose. The term “unit dose” when used in reference to a therapeutic composition refers to physically discrete units suitable as unitary dosage for the subject, each unit containing a predetermined quantity of active material calculated to produce the desired therapeutic effect in association with the required physiologically acceptable diluent, i.e., carrier, or vehicle.

Combination Therapy: Provided herein are methods for treating cancer, comprising administering a combination of at least two different agents (e.g., 2, 3, 4, 5, 6, 7, 8, 9, or 10 different agents). In one embodiment, the combination therapy comprises administration of at least one inhibitor of a blocking immune checkpoint molecule with at least one agonist of a stimulative checkpoint molecule and induction of fractional tissue damage. In another embodiment, the combination therapy comprises administration of at least one agonist of a stimulative immune checkpoint molecule in combination with fractional tissue damage (e.g., a fractional laser therapy treatment).

When at least two agents are administered as a combination therapy, they can be administered simultaneously. In other embodiments, the at least two agents are administered separately or concurrently. The agents can be delivered in any desired order by one of skill in the art. The immune checkpoint modulators can be administered intratumorally, systemically, orally or by any other desired forms of administration. In one embodiment, the anti-tumor response to combination therapy as described is synergistic.

### Efficacy Measurement

The efficacy of a treatment comprising one or more immune checkpoint modulators and/or the induction of fractional tissue damage that induces an immune response (e.g., a local intratumoral immune response and/or a systemic response, reduction in tumor or lesion size, improved sensitivity to treatment with a checkpoint inhibitor etc.) can be determined by the skilled clinician. However, a treatment is considered “effective treatment,” as the term is used herein, if any one or all of the signs or symptoms of, as but one example, cancer are altered in a beneficial manner, and/or other clinically accepted symptoms or markers of disease are improved or ameliorated, e.g., by at least 10% following treatment with an inhibitor. Efficacy can also be measured by failure of an individual to worsen as assessed by hospitalization or need for medical interventions (e.g., progression of the disease is halted or at least slowed). Efficacy in a population of patients can also be determined by measuring mortality rates due to advanced metastatic disease. Methods of measuring these indicators are known to those of skill in the art and/or described herein. Treatment includes any treatment of a disease in an individual or an animal (some non-limiting examples include a human, or a mammal) and includes: (1) inhibiting the disease, e.g., arresting, or slowing the progression of the cancer; or (2) relieving the disease, e.g., causing regression of symptoms; and (3) preventing or reducing the likelihood of the development of metastases.

The invention may be as described in any one of the following numbered paragraphs:
1. A method for inducing an immune response in a subject in need thereof, the method comprising: (a) administering an inhibitor of a blocking checkpoint molecule and an agonist of a stimulative checkpoint molecule to a subject in need thereof, and (b) treating a tissue of the subject with energy to induce fractional tissue damage, wherein an immune response is increased compared to the immune response produced by the inhibitor of the blocking checkpoint molecule and the agonist of the stimulative checkpoint molecule in the absence of the fractional tissue damage.
2. The method of paragraph 1, wherein the fractional tissue damage induces CD8+ T cell recruitment and/or activation.
3. The method of paragraph 1 or 2, wherein the energy that induces fractional tissue damage is selected from laser energy, ionizing radiation, ultrasound, and radio frequency energy.
4. The method of any one of paragraphs 1-3, wherein the blocking checkpoint molecule is selected from the group consisting of: PD-1, PD-L1, CTLA4, B7-H3, B7-H4, VISTA, TMIGD2, B7-H7, BTLA, HVEM, CD160, LAG3, TIGIT, CD96, CD155, TIM-3, Galectin-9, Adenosine, Adenosine A2a receptor, IDO, TDO, CEACAM1, SIRP alpha, CD47, CD200R and CD200.
5. The method of any one of paragraphs 1-4, wherein the stimulative checkpoint molecule is selected from the group consisting of: OX40, 4-1BB, GITR, CD28, ICOS, LIGHT, CD27, DNAM-1, 2B4, DC-SIGN, DR3, and CD40.
6. The method of any one of paragraphs 1-5, wherein the blocking checkpoint molecule is PD-1 and the stimulative checkpoint molecule is OX40.
7. The method of paragraph 6, wherein the PD-1 inhibitor and/or the OX40 agonist comprises an antibody.
8. The method of any one of paragraphs 1-7, wherein the cancer is colon cancer, lung cancer, melanoma, or breast cancer.
9. The method of paragraph 8, wherein the laser energy is emitted from a fractional CO₂ laser.
10. The method of any one of paragraphs 1-9, wherein the immune response comprises a local and/or systemic response.
11. A method for inducing an anti-tumor immune response in a subject in need thereof, the method comprising: (a) administering an inhibitor of a blocking checkpoint molecule and an agonist of a stimulative checkpoint molecule to a subject having cancer, and (b) treating tumor tissue of the subject with energy to induce fractional tissue damage, wherein an anti-tumor immune response is increased compared to the anti-tumor immune response produced by the inhibitor of the blocking checkpoint molecule and the agonist of the stimulative checkpoint molecule in the absence of the fractional tissue damage.
12. A method for treating cancer in a subject in need thereof, the method comprising: (a) administering an inhibitor of a blocking checkpoint molecule, and an agonist of a stimulative checkpoint molecule to a subject having cancer, and (b) treating tumor tissue of the subject with energy to induce fractional tissue damage, thereby treating cancer in the subject.
13. The method of paragraph 11 or paragraph 12, wherein the energy that induces fractional tissue damage is selected from laser energy, ionizing radiation, ultrasound, and radio frequency energy.
14. The method of any one of paragraphs 11-13, wherein the energy that induces fractional tissue damage is laser energy.
15. The method of any one of paragraphs 11-14, wherein the blocking checkpoint molecule is selected from the group consisting of: PD-1, PD-L1, CTLA4, B7-H3, B7-H4, VISTA, TMIGD2, B7-H7, BTLA, HVEM, CD160, LAG3, TIGIT, CD96, CD155, TIM-3, Galectin-9, Adenosine, Adenosine A2a receptor, IDO, TDO, CEACAM1, SIRP alpha, CD47, CD200R and CD200.
16. The method of any one of paragraphs 11-15, wherein the stimulative checkpoint molecule is selected from the group consisting of: OX40, 4-1BB, GITR, CD28, ICOS, LIGHT, CD27, DNAM-1, 2B4, DC-SIGN, DR3, and CD40.
17. The method of any one of paragraphs 11-16, wherein the blocking checkpoint molecule is PD-1 and the stimulative checkpoint molecule is OX40.
18. The method of paragraph 17, wherein the PD-1 inhibitor and/or the OX40 agonist comprises an antibody.
19. The method of any one of paragraphs 11-18, wherein the cancer is colon cancer, lung cancer, melanoma, or breast cancer.
20. The method of paragraph 19, wherein the laser energy is emitted from a fractional CO₂ laser.
21. The method of any one of paragraphs 11-20, wherein the anti-tumor immune response or the treatment of cancer comprises induction of CD8+ T cells.
22. The method of any one of paragraphs 11-21, wherein the anti-tumor immune response comprises a systemic response.
23. The method of paragraph 22, wherein the anti-tumor immune response induces an abscopal effect against a tumor that is not treated with the fractional laser to induce fractional tissue damage.
24. The method of any one of paragraphs 11-23, wherein the anti-tumor immune response prevents or reduces the likelihood of cancer recurrence.
25. The method of any one of paragraphs 11-24, wherein the anti-tumor immune response increases progression-free survival, reduces the size of one or more tumors, and/or increases overall response rate.
26. A method for inducing an anti-tumor immune response in a subject in need thereof, the method comprising: (a) administering an OX40 agonist to a subject having cancer, and (b) treating tumor tissue of the subject with energy to induce fractional tissue damage, wherein the anti-tumor immune response is increased compared to the anti-tumor immune response produced by the OX40 agonist of in the absence of the fractional tissue damage.
27. A method for treating cancer in a subject, the method comprising: (a) administering an OX40 agonist to a subject having cancer, and (b) treating tumor tissue of the subject with energy to induce fractional tissue damage, thereby treating cancer in the subject.
28. The method of paragraph 26 or 27, further comprising administering an inhibitor of a blocking checkpoint molecule selected from the group consisting of: PD-1, PD-L1, CTLA4, B7-H3, B7-H4, VISTA, TMIGD2, B7-H7, BTLA, HVEM, CD160, LAG3, TIGIT, CD96, CD155, TIM-3, Galectin-9, Adenosine, Adenosine A2a receptor, IDO, TDO, CEACAM1, SIRP alpha, CD47, CD200R and CD200.
29. The method of any one of paragraphs 26-28, wherein the blocking checkpoint molecule is PD-1.
30. The method of paragraph 29, wherein the PD-1 inhibitor and/or the OX4 agonist comprises an antibody.
31. The method of any one of paragraphs 26-30, wherein the cancer is colon cancer, lung cancer, melanoma, or breast cancer.
32. The method of any one of paragraphs 26-31, wherein the energy that induces fractional tissue damage is selected from laser energy, ionizing radiation, ultrasound, and radio frequency energy.
33. The method of any one of paragraphs 26-32, wherein the fractional laser comprises a fractional CO₂ laser.
34. The method of any one of paragraphs 26-33, wherein the anti-tumor immune response or treatment of cancer comprises induction of CD8+ T cells, increase in number of CD8+ T cells, or activation of CD8+ T cells.
35. The method of any one of paragraphs 26-34, wherein the anti-tumor immune response or treatment of cancer comprises a systemic response.
36. The method of any one of paragraphs 26-35, wherein the anti-tumor immune response or treatment of cancer induces abscopal treatment of a tumor that is not treated with energy emitted from the fractional laser to induce fractional tissue damage.
37. The method of any one of paragraphs 26-36, wherein the anti-tumor immune response prevents or reduces the likelihood of cancer recurrence.
38. The method of any one of paragraphs 26-37, wherein the anti-tumor immune response increases progression-free survival, reduces the size of one or more tumors, and/or increases overall response rate.
39. A system for inducing an anti-tumor immune response or treating cancer in a subject, the system comprising: a device configured to induce fractional tissue damage and means for administering an inhibitor of a blocking checkpoint molecule, and an agonist of a stimulative checkpoint molecule.
40. The system of paragraph 39, wherein the device configured to induce fractional tissue damage comprises a fractional laser, radiofrequency energy, or focused ultrasound.
41. The system of paragraph 40, wherein the fractional laser is an ablative fractional laser.
42. The system of paragraph 41, wherein the fractional laser is a fractional CO₂ laser.
43. A method of inducing pyroptosis in tumor cells in a subject, the method comprising: (a) treating tumor tissue of a subject with energy to induce fractional tissue damage; and (b) administering an inhibitor of a blocking checkpoint molecule and an agonist of a stimulative checkpoint molecule to the subject, wherein pyroptosis is induced in tumor cells in the subject.
44. The method of paragraph 43, wherein pyroptosis is induced at a site separate from the tumor tissue treated with energy to induce fractional tissue damage.
45. A method for inducing an anti-tumor immune response in a subject in need thereof, the method comprising: (a) administering one or more agents to a subject having cancer, and (b) treating tumor tissue of the subject with energy to induce fractional tissue damage, wherein the number of CD8+ T cells or activated CD8+ T cells is increased in the tumor compared to the anti-tumor immune response produced by the one or more agents in the absence of the fractional tissue damage.
46. A method for treating cancer in a subject, the method comprising: (a) administering one or more agents to a subject having cancer, and (b) treating tumor tissue of the subject with energy to induce fractional tissue damage, wherein the number of CD8+ T cells or activated CD8+ T cells is increased in the tumor, thereby treating the cancer in the subject.

### Examples

Laser based cancer treatment has the potential to become a breakthrough medical technology, which promises to not only translate into a financially profitable product for U.S. universities, medical institutions and companies, but will also help to make treatment for cancer patients more effective, less painful, and more affordable. The currently available cancer treatment options face the challenge of being either too invasive, too costly and/or require extremely complex technological tools. Making a contribution to better cancer treatments through laser induced immunity improvement could massively change the currently available options for patients and therefore help extend life expectancy and quality for the 14.5 million cancer patients currently in the U.S., as well as internationally.

Use of fractional damage-producing physical device+blocking checkpoint molecules and/or stimulative checkpoint molecules is expected to be tremendously beneficial in the world. Commercial products and applications include anti-PD-1 inhibitors and OX40 agonists or other checkpoint molecules with indications for use in combination and with fractional tissue damage for cancer therapy and/or as adjuvant to vaccination.

The inventors have found that fractional laser and fractional radiofrequency energy can boost immune response of immune blocking checkpoint molecule such as anti PD-1 inhibitor and stimulative checkpoint molecules such as OX40 agonist, 4-1BB agonist, GITR agonist or CD40 agonist. The key point is “fractional damage.” The boosting effect was not produced by a device which created damage of whole tumor. That is, a fractional damage pattern is necessary to boost the immune response.

Additionally, fractional damage produced by other physical devices, such as fractional radiation (e.g. ionizing radiation), fractional ultrasound, fractional radiofrequency energy and the like, are also expected to produce a similar immune-boosting phenomenon. In addition to anti-PD-1 inhibitors and OX40 agonists, other blocking checkpoint molecules (PD-L1, CTLA4, B7-H3, B7-H4, VISTA, TMIGD2, B7-H7, BTLA, HVEM, CD160, LAG3, TIGIT, CD96, CD155, TIM-3, Galectin-9, Adenosine, Adenosine A2a receptor, IDO, TDO, CEACAM1, SIRP alpha, CD47, CD200R and CD200 blocking antibody) and other stimulative checkpoint molecules (4-1BB, GITR, CD28, ICOS, LIGHT, CD27, DNAM-1, 2B4, DC-SIGN, DR3, and CD40 agonist) are specifically contemplated for use with a device that induces fractional damage.

As proof of concept, the inventors have focused on developing laser-based treatments for cancer therapy. Specifically, they have worked to develop a highly innovative method of using ablative fractional photothermolysis (aFP), a laser-assisted treatment producing a pattern of microscopic treatment zones (MTZs) in biological tissue that is used in dermatological treatments, for non-cancer therapy. It was found that aFP could be used for cancer therapy and was observed to induce innate, adaptive immunity and promote regression of a remote and untreated cancer as abscopal effect, despite the small degree of thermal injury it inflicted to the overall cancer and the absence of any introduced drug or other bioactive substance. Moreover, it was discovered that aFP cancer treatment induced long-term immune memory and prevented cancer recurrence. Furthermore, the inventors found that combining aFP therapy with an anti-PD-1 inhibitor and OX40 agonist or other checkpoint molecules which boosted the expansion, proliferation, activation and effector function of cytotoxic T lymphocyte by blocking the PD-1/PD-L1 axis and stimulating receptor of immune checkpoint molecules (4-1BB, GITR and CD40 agonist) further boosted systemic immunity against cancer. Thus, such a therapeutic strategy can achieve significant efficacy in poorly immunogenic cancers of the colon, breast, lung and other organs. Stimulation of the immune response can also be expected to boost the immune response when vaccination is given where the tissue in proximity of the vaccination is treated with fractional laser or other devices providing fractional damage to the tissue as described in the attached documents.

Currently, ablative fractional photothermolysis (aFP) with CO₂ laser is used for a wide variety of dermatological indications. This study presents and discusses the utility of aFP for treating oncological indications. The inventors used a fractional CO₂ laser and checkpoint molecule modulating agents, such as an anti-PD-1 and OX40 antibody, to treat a tumor established bilaterally by the CT26 wild type (CT26WT) colon carcinoma cell line, treating tumors on one-side and observing both tumors. Interestingly, tumors in triple treatments such as aFP+anti-PD-1+anti-OX40 group grew significantly slower and complete remission was observed for tumors on both aFP-treated and untreated sides. Flow cytometric analysis showed the triple treatments elicited an increase of CD8+ and granzyme B+CD8+ T cells. Triple therapy-mediated tumor regression and survival was abrogated when anti-CD8 antibody was used for ablation of the CD8+ T cells to investigate whether CD8+ T cells play critical role for therapeutic benefit. And when using two bilaterally mismatched cancer cells-inoculated mice, the effect of the triple therapy on the non-aFP-treated tumor was abrogated, showing antigen specificity was also important for the treatment. This study has demonstrated a potential role of aFP treatments in oncology.

Ablative fractional photothermolysis (aFP) can be characterized as laser-assisted treatment generating a pattern of microscopic treatment zones (MTZs) as small zones of thermally damaged tissue in biological tissue¹ and additionally produces a central “hole” of physically-removed (ablated) tissue, surrounded by a small cuff of thermally-damaged tissue in MTZs^{2,3}. In general, the width or diameter of the MTZs measures less than 0.5 mm. aFP techniques typically expose only a small fraction of the tissue (often an areal fraction of approximately 5-27%), leaving the majority of tissue spared or unexposed.⁴ aFP is used for a large range of dermatological indications, such as treatment of photodamaged skin, dyschromia, rhytides, and different kind of scars including acne, surgical and burn scars^{1,5-8}. However, aFP is not used for cancer treatment and no studies to date have investigated its potential for cancer therapy. The inventors investigated if aFP can be used for induction of anti-tumor immunity locally and systemically, and whether it can promote regression of a remote and untreated tumor.

Next, the inventors examined use of an OX40 agonist whose effect is reported to include promotion of the expansion, proliferation, activation and effector function of killer CD8 and helper CD4 T cells and exertion of their anticancer activity by depleting the number of Treg cells. Thus, the inventors have investigated whether OX40 agonist could boost effects of aFP+anti-PD-1 combination therapy and then induce anti-tumor immunity systemically and promote regression of a remote and untreated tumor.

### Results

aFP Treatment Boosts Effect of Systemic Anti-Tumor Immunity Induced by Anti-PD-1 Inhibitor and OX40 Agonist Combination Therapy.

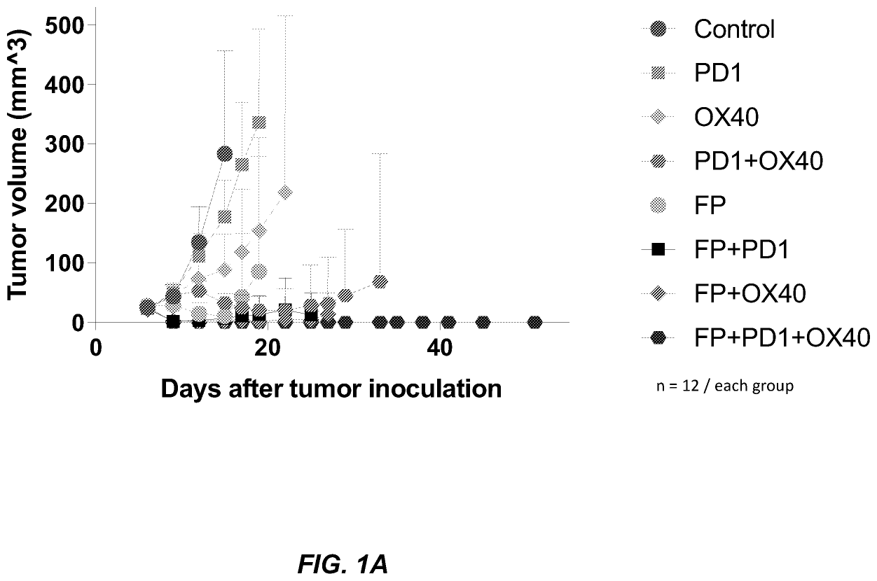

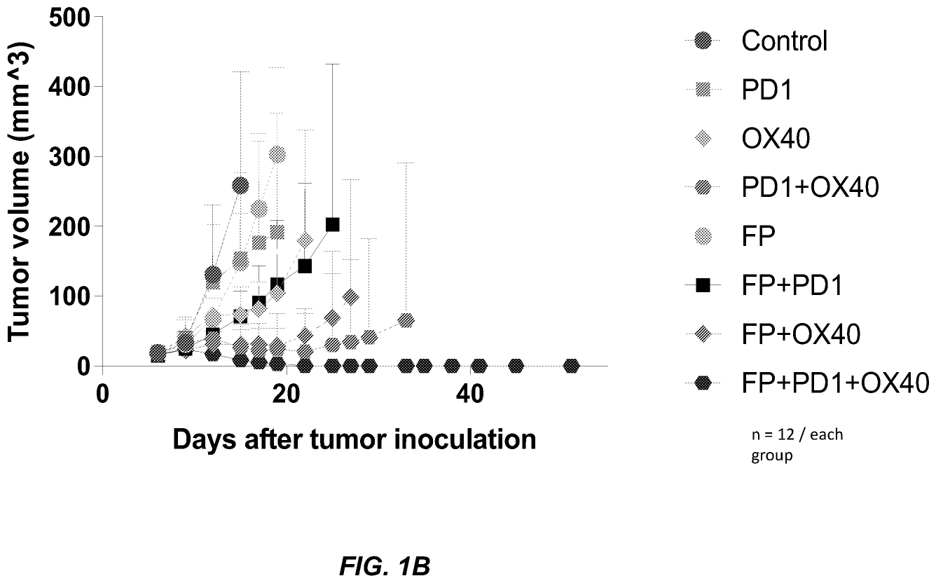

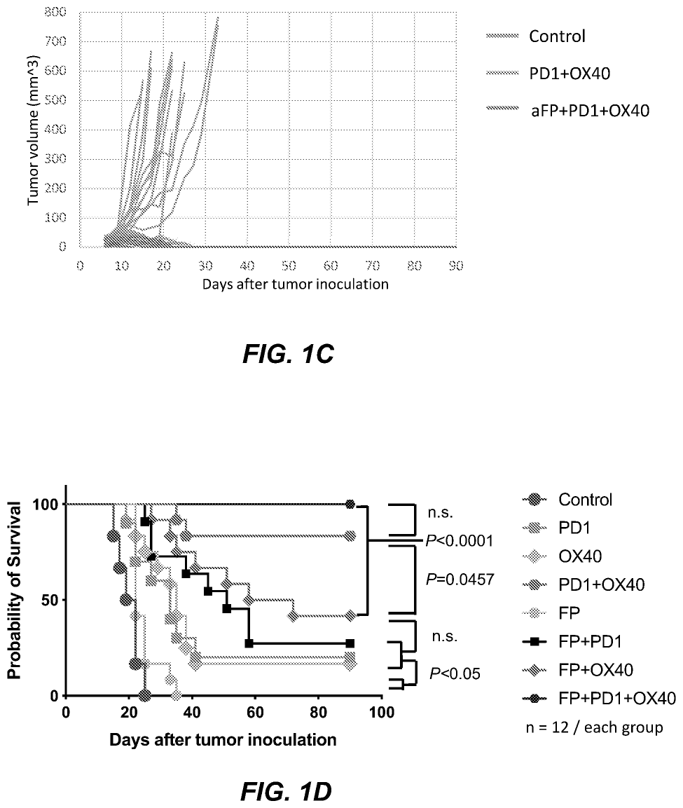

To investigate whether aFP treatment with immune checkpoint molecules could induce systemic anti-tumor immunity efficiently, the inventors established a two tumor mouse model, where the mice have a tumor on both hind legs (with the aFP-treated tumor on either leg) and the growth of both tumors is monitored. aFP laser irradiation was performed 6 days after tumor inoculation. At this time, the tumors have reached a typical diameter of about 4 mm. Triple therapy (e.g., aFP+anti-PD-1+anti-OX40) significantly led to a reduction in tumor volume and growth rate after the treatment (FIGS. 1A-1C). The triple therapy also led to complete remission of all tumors and all of the mice survived more than 90 days in the group (FIG. 1D). The significance value for the difference between the survival curves are shown in Table 1.

TABLE 1
Significance value for the difference between the survival curves (Treated on day 6)

| | | | | Anti-PD-1 + | | aFP + | aFP + |
|---|---|---|---|---|---|---|---|
| | Control | anti-PD-1 | OX40 | OX40 | aFP | anti-PD-1 | OX40 |
| anti-PD-1 | P = 0.0007 | | | | | | |
| OX40 | P < 0.0001 | n.s | | | | | |
| Anti-PD-1 + OX40 | P < 0.0001 | P = 0.001 | P = 0.0007 | | | | |
| aFP | P = 0.0217 | P = 0.0281 | P = 0.0018 | P < 0.0001 | | | |
| aFP + anti-PD-1 | P < 0.0001 | n.s. | n.s. | P = 0.01 | P < 0.0001 | | |
| aFP + OX40 | P < 0.0001 | n.s. | n.s. | P = 0.0457 | P < 0.0001 | n.s. | |
| aFP + anti-PD-1 + OX40 | P < 0.0001 | P < 0.0001 | P < 0.0001 | n.s. | P < 0.0001 | P = 0.0002 | P < 0.0001 |

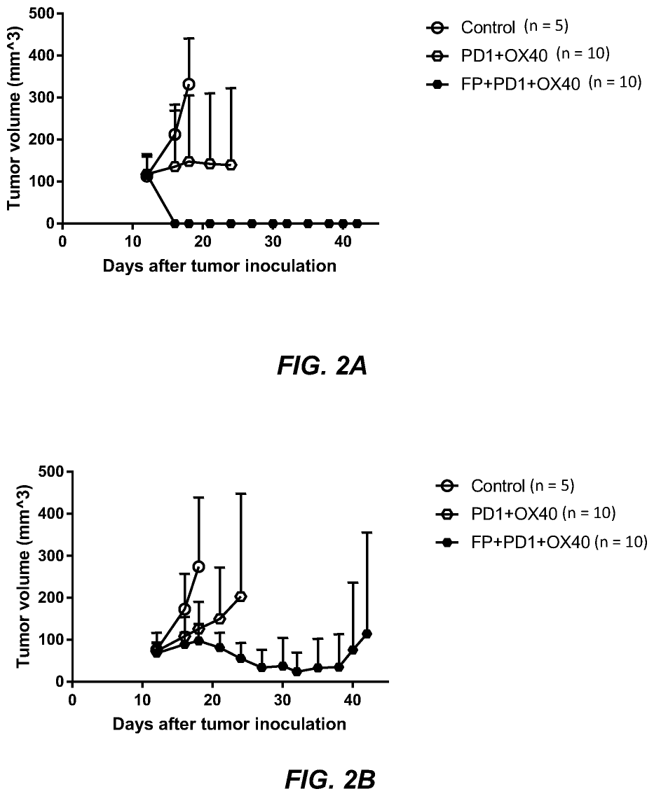

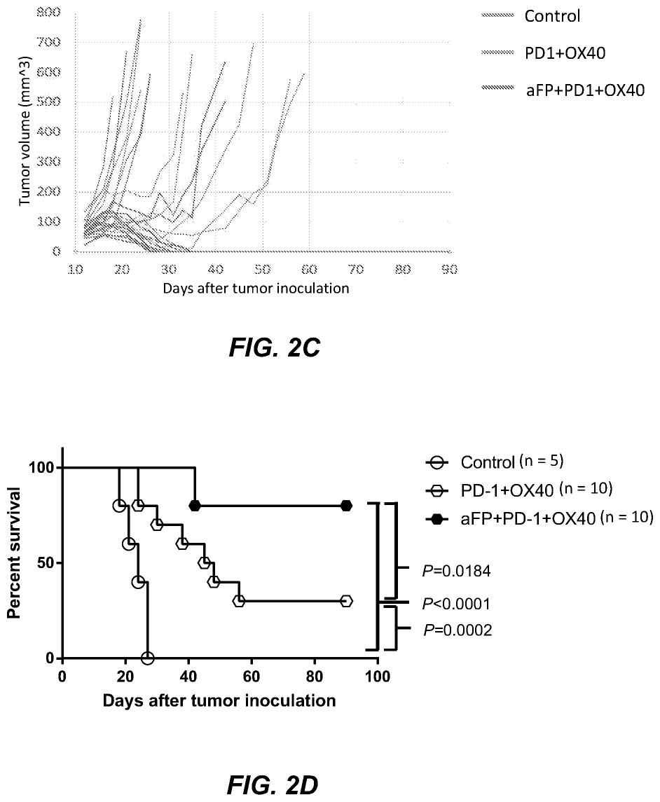

However, there was no significant difference between the triple therapy and double therapy (e.g., anti-PD-1+anti-OX40) group. Therefore, the inventors compared these two groups in a model having larger tumor; that is, in which treatment was initiated 12 days after tumor inoculation. At this time, the tumors have reached a typical diameter of about 7 mm. aFP treatment was performed on day 12 and anti-PD-1 inhibitor and/or OX40 agonist were administered on days 12, 14, 16, 18, and 20 after tumor cell inoculation. The triple therapy led to a greater reduction in tumor volume and growth rate than the double therapy (FIGS. 2A-2C). Moreover, tumors on both sides shrank completely and 8 of the 10 mice in the triple therapy group survived more than 90 days, while such shrinkage occurred in only 3 of the 10 mice in the double therapy group (FIG. 2D). The significance value for the difference between the survival curves are shown in Table 2.

TABLE 2
Significance value for the difference between
the survival curves (treated on day 12)

| | Control | Anti-PD-1 + OX40 |
|---|---|---|
| Anti-PD-1 + OX40 | P = 0.0002 | |
| aFP + anti-PD-1 + OX40 | P < 0.0001 | P = 0.0184 |

### Surviving Mice Develop Long Term Anti-Tumor Immunity

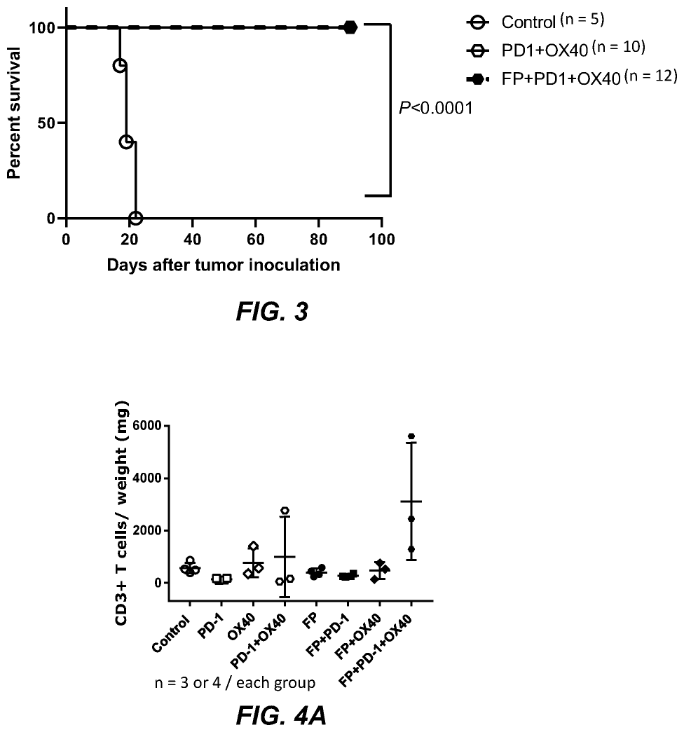

To evaluate the production of any induced long term anti-tumor immunity, the inventors performed a rechallenge experiment following the treatment. Ten mice in the double therapy (i.e., anti-PD-1+anti-OX40 group) and twelve mice in the triple therapy group (i.e., aFP+anti-PD-1+anti-OX40) which survived for more than 90 days after the treatment, were subsequently inoculated subcutaneously with 3.5×10⁵ CT26WT cells in right thigh (aFP-untreated thigh in case of aFP-treated mouse). Five age-matched naive mice were inoculated with the same number of CT26WT cells, respectively, in the right thigh as a control. The tumor on the naive mice in the control groups progressed over time. However, tumors on the rechallenged mice in two groups did not appear to progress (FIG. 3). They remained tumor-free for at least another 60 days following the inoculation (FIG. 3). The significance values for the differences between the survival curves are: control vs. anti-PD-1+anti-OX40 (p<0.0001), control vs. aFP+anti-PD-1+anti-OX40 (p<0.0001).

The Triple Therapy Significantly Induces Tumor-Infiltrating CD8+ and Granzyme B+CD8+T Lymphocytes in aFP-Untreated Contralateral Tumors.

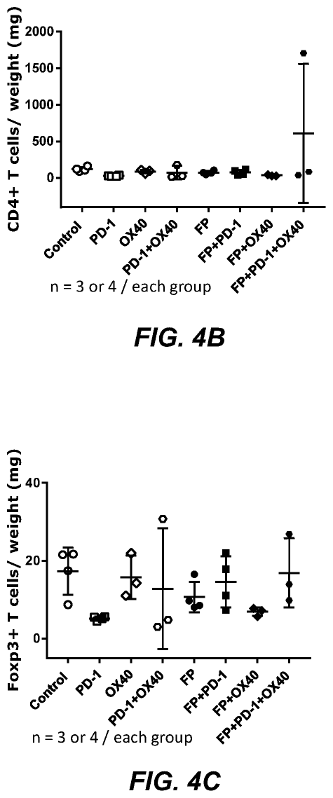

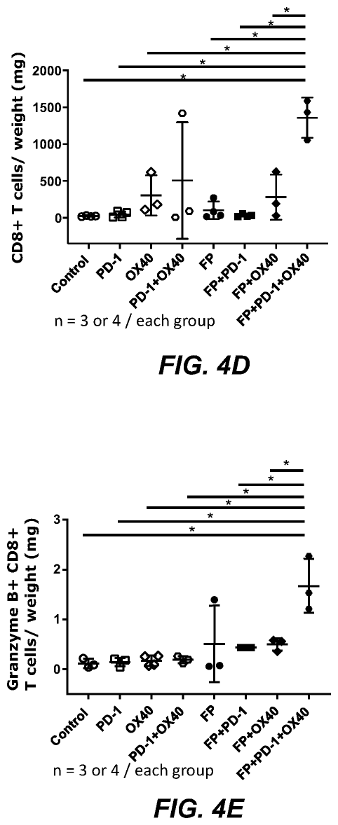

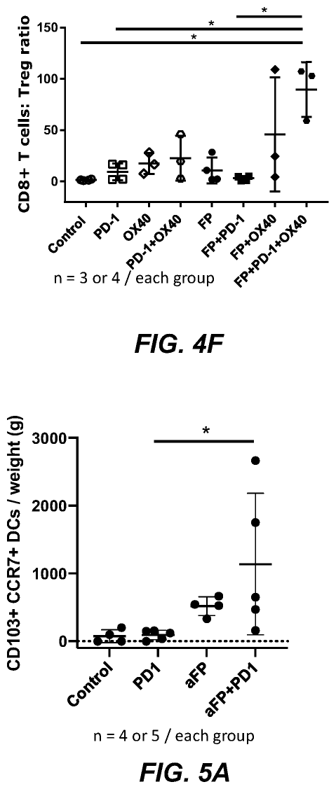

To investigate why the aFP-untreated contralateral tumor in the triple therapy, aFP+anti-PD-1+anti-OX40 group, shrunk much more than rest of the groups, the inventors measured the number of tumor-infiltrating CD3+, CD4+, CD8+, and granzyme B+CD8+ T cells and Foxp3+ regulatory T cells (Tregs) in aFP-untreated contralateral tumor 5 days after treatment started using flow cytometry. It was found that CD8+ and granzyme B+CD8+ T cells increased per tumor weight in the triple therapy group (CD8/weight: control, anti-PD-1, anti-OX40, aFP, aFP+anti-PD-1 or aFP+anti-OX40 vs. aFP+anti-PD-1+anti-OX40 (p<0.05; FIG. 4D); granzyme B+CD8/weight: control, anti-PD-1, anti-OX40, anti-PD-1+anti-OX40, aFP+anti-PD-1 or aFP+anti-OX40 vs. aFP+anti-PD-1+anti-OX40 (p<0.05; FIG. 4E) though there is no significant difference between all groups regarding CD3+ and CD4+ T cells (FIGS. 4A-4B). Moreover, there was no significant difference regarding Treg numbers among the groups (FIG. 4C), resulting in increase of ratio of CD8+ T cells to Treg in triple therapy group. (control, anti-PD-1, or aFP+anti-PD-1 vs. aFP+anti-PD-1+anti-OX40 (p<0.05; FIG. 4F).

aFP+Anti-PD-1 Therapy Increases CD103+CCR7+ Dendritic Cells (DCs) in aFP-Treated Tumor and XCR1+DCs in Drainage Lymph Node.

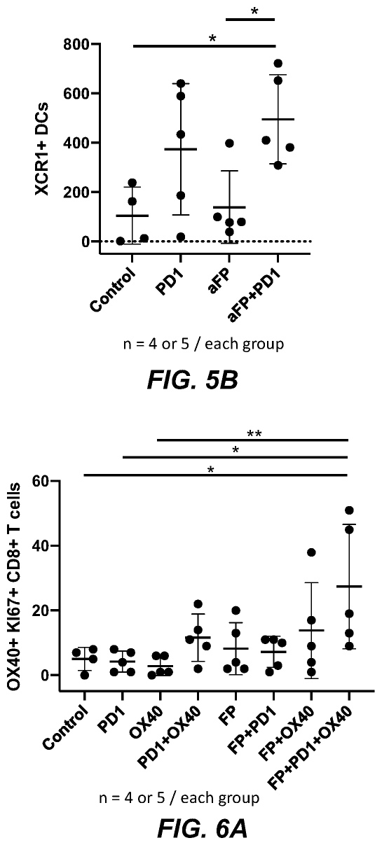

To investigate why the triple therapy induced tumor-infiltrating CD8+T lymphocytes in aFP-untreated contralateral tumors significantly, the inventors measured the number of DCs (CD45+CD11c+MHC II+) in aFP-treated tumor and in drainage lymph node at aFP-treated side on 3 days after treatment using flow cytometry. To measure the number, the inventors designated four groups: control, PD-1, aFP and aFP+PD1 groups. In this setting the OX40 agonist was not used due to the absence of expression of OX40 on DCs. First, the inventors measured DC expressing CD103 and CCR7 in aFP-treated tumors since CD103+DCs are as the population with the capability to induce proliferation of naive CD8 T cells (22). DC migration to LNs is mediated by interaction between CCR7 chemokine receptor expressed on CD103+DCs (23), and CCL19 and CCL21 which are chemoattractant for CCR7 from LNs (24). It was found that there was a trend indicating that the number of CD103+CCR7+DCs per tumor weight increased in groups with aFP treatment such as aFP and aFP+PD1 groups (FIG. 5A). aFP+anti-PD-1 group showed significant difference as compared to anti-PD-1 group (p<0.05). Next, the inventors measured the number of DCs expressing XCR1 in drainage lymph node at aFP-treated side since XCL1, which is a ligand of XCR1, is secreted from CD8+ T cells and is a highly specific chemoattractant for XCR1+DC subset. The XCR1-XCL1 axis leads to promotion of interaction between cross-presenting DCs and CD8+ T cells to enhance cross-priming, meaning the axis is an integral component in the development of efficient cytotoxic immunity (25, 26). As shown in FIG. 5B, XCR1+DCs increased in aFP+anti-PD-1 group significantly comparing with control group and aFP group (control or aFP vs. aFP+anti-PD-1: p<0.05). Taken together, these data indicate that aFP treatment recruits DCs with the capability to induce proliferation of naive CD8 T cells, and migrate to drainage lymph node in aFP-treated tumor and anti-PD-1 inhibitor increases DCs with an integral component in the development of efficient cytotoxic immunity.

The Triple Therapy Induces Expression of OX40 and PD-1 on CD8+ T Cells in Drainage Lymph Node at aFP-Treated Side Significantly.

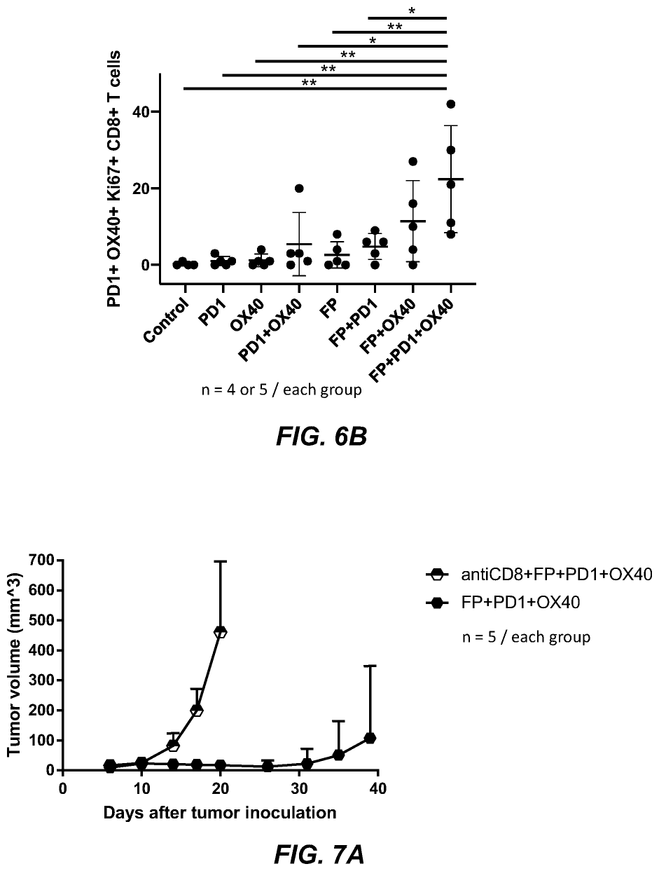

Furthermore, to investigate why the triple therapy induced tumor-infiltrating CD8+T lymphocytes in aFP-untreated contralateral tumors significantly, the inventors measured expression of PD-1, OX40, and Ki67 on CD8+ T cells in drainage lymph node at aFP-treated side 5 days after treatment using flow cytometry. OX40 and PD-1 are expressed rapidly after antigen stimulation on CD8+ T cells (27, 28) meaning expression of OX40 and PD-1 are markers of acquisition of the stimulation. Moreover, OX40+CD8+ T cells have potential to be proliferated due to OX40 agonist and suppressive effect of PD-1/PD-L1 axis can be prevented by anti-PD-1 inhibitor if OX40+CD8+ T cells express PD-1. Strikingly, it was found that OX40+CD8+ T cells with Ki67, which is a proliferation marker that is significantly increased in the triple therapy group (anti-OX40 vs aFP+anti-PD-1+anti-OX40: p<0.01, control or anti-PD-1 vs. aFP+anti-PD-1+anti-OX40: p<0.05; FIG. 6A). Moreover, PD-1+OX40+ki67+CD8+ T cells were significantly increased in the triple therapy group, meaning that the triple therapy induced antigen-stimulated CD8+ T cells with potential to be proliferated by OX40 agonist and affected by anti-PD-1 inhibitor for migration to tumors (control, anti-PD-1, anti-OX40, or aFP, vs. aFP+anti-PD-1+anti-OX40 (p<0.005), anti-PD-1+anti-OX40 or aFP+anti-PD-1 vs. aFP+anti-PD-1+anti-OX40 (p<0.05; FIG. 6B).

Adaptive Immunity Plays a Role in Eradication of Cancer Cells after Triple Therapy

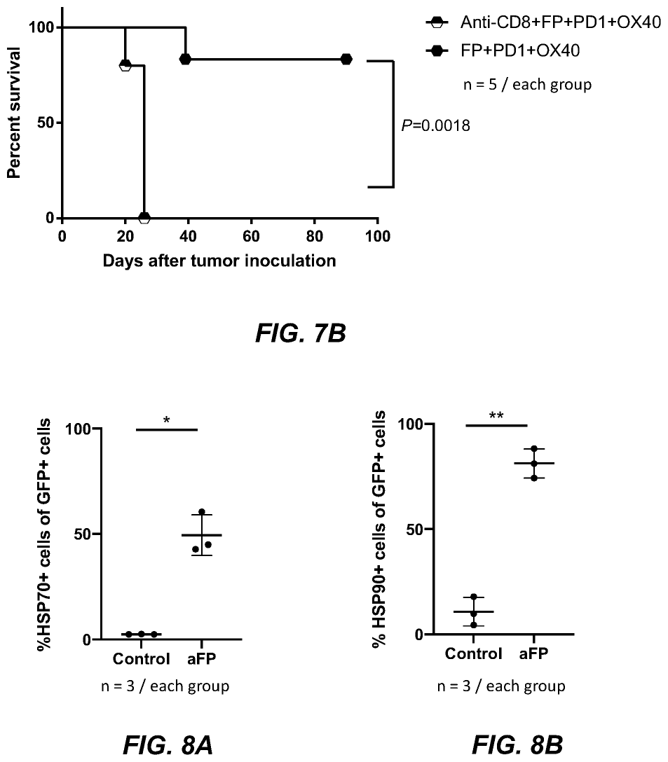

To investigate whether adaptive immunity is necessary to eradicate cancer cells after triple therapy (aFP+anti-PD-1+anti-OX40), the experiment was repeated with anti-CD8 depletion antibody to ablate CD8+ T cells in the mouse. As shown in FIG. 5, treatments with the depletion antibody abrogate tumor regression and survival mediated by the triple therapy, while 4 out of 5 mice survived in the group with no CD8 depletion. (p=0.0018; FIG. 7). This observation indicates adaptive immunity plays an important role in the therapeutic benefit of the triple therapy.

### aFP Treatment Induces Expression of HSP70 and 90 and Calreticulin on the Tumor Cell.

There are reports which show extracellular localized and membrane-bound HSPs play key roles in eliciting antitumor immune responses by acting as carriers for tumor-derived immunogenic peptides, as adjuvants for antigen presentation, or as targets for the innate immune system (29). In the present study, RNA sequence reveal upregulation of HSPa1a and HSPa1b which are gene of HSP70 in aFP-treated tumor 24 hours after the irradiation (Table 3).

TABLE 3
Data of RNA sequence (aFP vs control group

| | log2FoldChange(aFP/ | | Up/Down- |
|---|---|---|---|
| Symbol | Control) | Probability | Regulation(aFP/Control) |
| Hspa1a | 7.725630695 | 1 | Up |
| Hspa1b | 8.309049618 | 0.99999999 | Up |
| II1a | 3.746162754 | 0.99253465 | Up |
| II1b | 3.446302695 | 0.999999807 | Up |
| Ifnb1 | 4.009460329 | 0.987913968 | Up |

To investigate the up-regulation of IL-1 and IFN-beta in the RNA level, the inventors measured RNA expression of IL-1 and IFN-beta in the tumor using RNA sequence analysis. aFP-irradiated tumor and non-irradiated tumor as control was harvested 24 hours after irradiation, RNA was extracted using RNeasy mini kit (Qiagen) and RNA sequencing was performed by commercial company (Beijing Genomics Institute). Each group contains 3 tumors respectively. The table shows aFP-irradiated tumor induce IL-1 and IFN-beta expression.

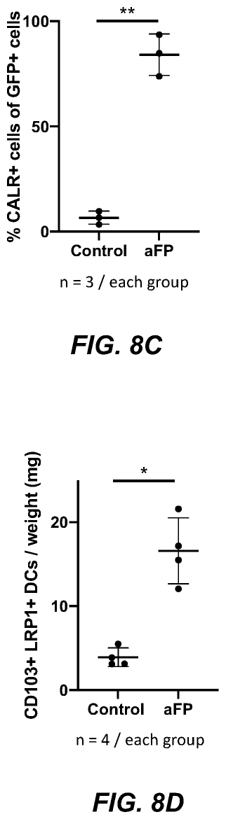

Therefore, in addition to HSP90 the inventors measured HSP70 protein expression on the aFP-treated tumor 3 days after the irradiation using flow cytometry. Moreover, the inventors measured tumor expressing calreticulin (CALR), which is the so-called “eat me” sign (30) and CD103+DCs expressing LRP1, which is a receptor for HSP70, 90 and CALR. To measure the expression of HSP70, 90 and CALR on tumors, the inventors transfected green fluorescence protein (GFP) into CT26WT colon carcinoma as a marker for detection. As shown in FIGS. 8A-8C, the expression of HSP70, 90, and CALR on the carcinoma cells in aFP group was significantly greater than the expression in the control group. In addition, CD103+DCs expressing LRP1 increased in aFP-treated tumor significantly (FIG. 8D). Therefore, these data indicate that aFP induces expression of molecules which potentially promote anti-tumor immunity on the tumor.

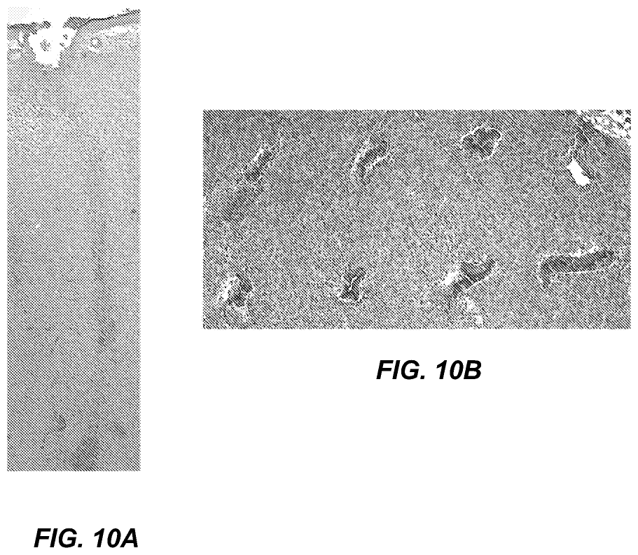

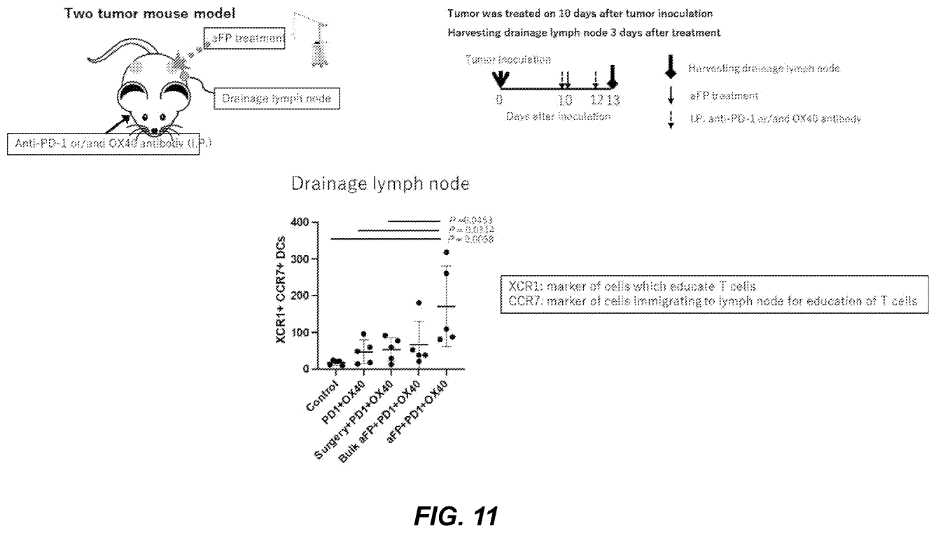

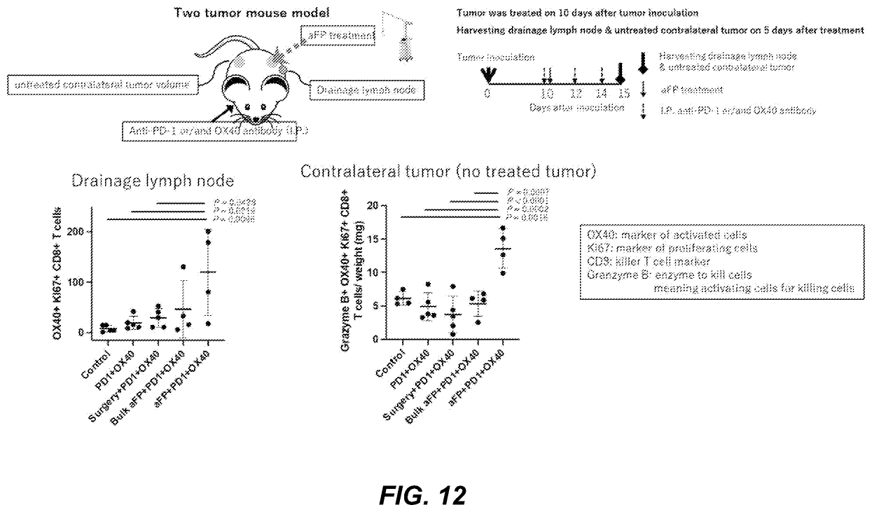

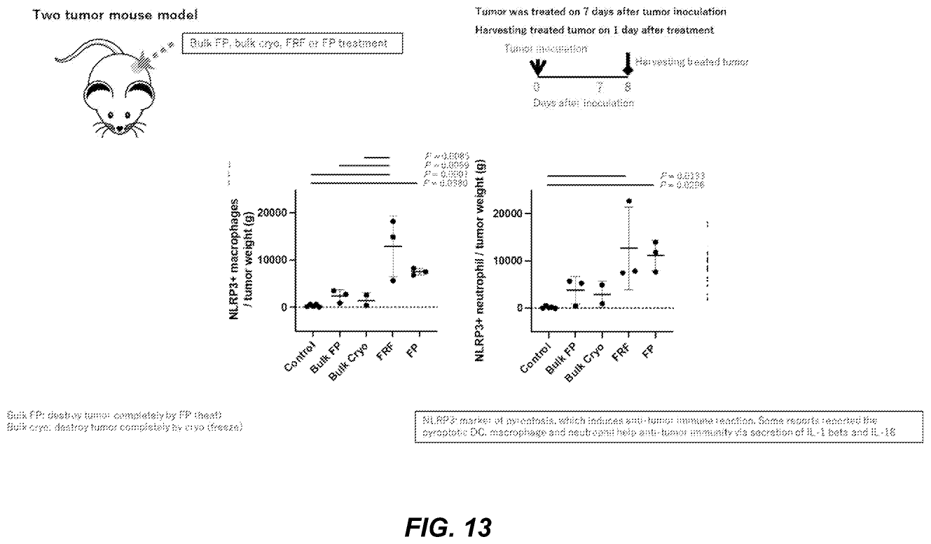

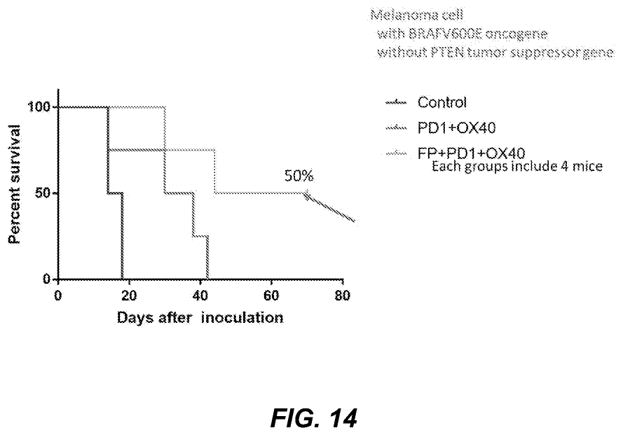

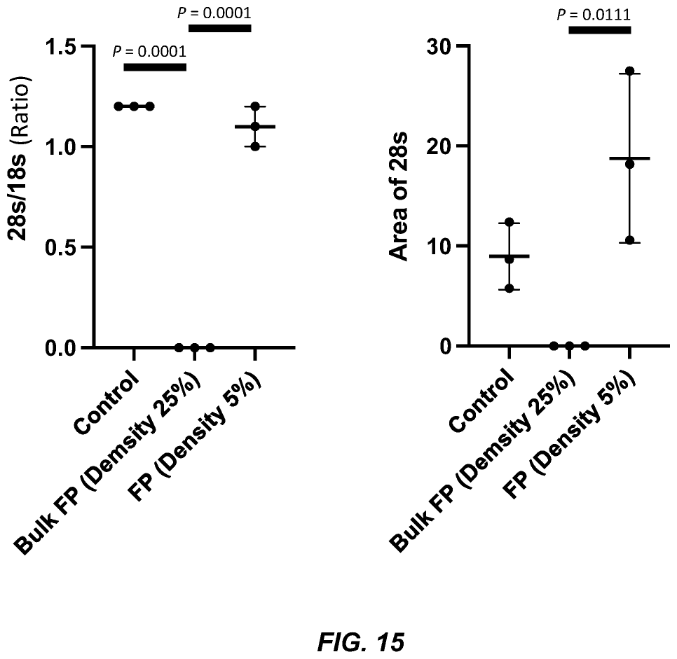

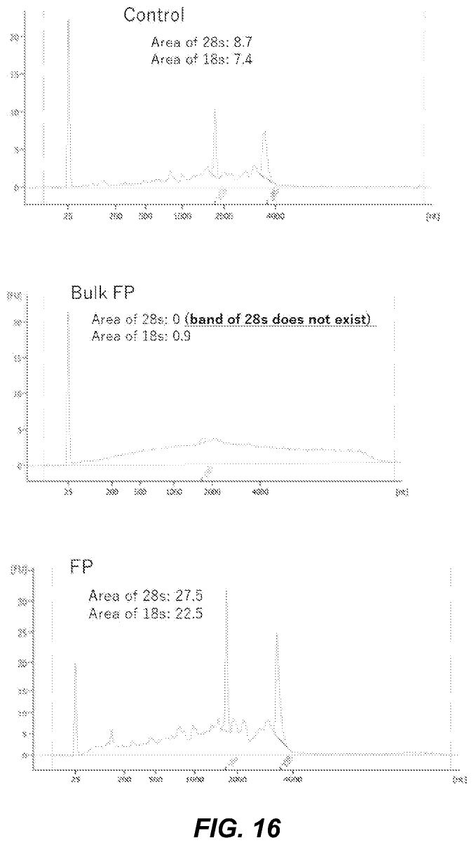

In this study, the inventors demonstrate a significant tumor size reduction and initiation of a systemic, tumor specific immune response induced by triple therapy using aFP+anti-PD-1+OX40 agonist. A large range of clinical indications currently make use of aFP, yet the ability of aFP to induce tumor regression is unexpected, because aFP treatments are generally applied to achieve localized tissue effects within the treatment area such as in the stimulation of wound healing and tissue regeneration. There are reports that utilize tumor associated antigen (TAA) properties of the tumor for cancer therapy inducing anti-tumor activity (31). However, the antigen expression on the cell in a normal state is not sufficient to stimulate an immune response to prevent tumor growth (32). Therefore, in a previous study, the inventors employed aFP to produce thermal denaturation, potentially releasing TAA and enhancing anti-tumor immunity in order for the cytotoxic T cells to recognize and bind the immunodominant peptide epitope derived from the antigens on MHC as it simultaneously creates MTZ with intact interspersed tissue(10, 11). The total MTZ/tumor volume ratio was adjusted to approximately 5% of inoculated tumors (FIG. 10), thereby aiming to enhance inflammation while avoiding the direct killing of the tumor bulk (10, 11). Next, the inventors found that aFP treatment against the CT26WT tumors could induce anti-tumor immunity and produce improvement in long-term survival (11). The CT26WT colon carcinoma cells used in the study have TAA in a single peptide known as AH-1, a non-mutated nonamer derived from the envelope protein (gp70) of an endogenous ecotropic murine leukemia provirus (33). In the previous study, pentamer staining revealed an increase of TAA (AH1) specific CD8+ T cells per tumor weight in the aFP-treated groups(11). The result indicates that aFP can release TAA and induce anti-tumor immunity. The current study showed that levels of the antigen-presenting CD103+CCR7+DCs increased in aFP-treated tumor in groups with aFP treatment.

OX40 is a secondary co-stimulatory immune checkpoint molecule, which is expressed predominantly by T cells during antigen-specific priming under the presence of inflammatory cytokines such as Interleukin-1 (II-1) (28). The ligand for OX40 such as OX40L (CD252) is expressed on activated professional antigen-presenting cells such as dendritic cells (DCs), macrophages, and B cells (43, 44). Costimulatory signals via OX40/OX40L axis to T cell promotes the expansion, proliferation (12-14), activation and effector function of killer T cells and helper T cells (15-17). Moreover, some studies have also shown that OX40 agonists might exert their anticancer activity by depleting the number of Foxp3+Treg cells (18-21). In the present study, though Treg depletion could not be observed, CD8+ T cells increased in triple therapy such as aFP+anti-PD-1+OX40 agonist group and the complete remission of all tumor was observed in case treatment started 6 days after tumor inoculation. Without wishing to be bound by theory, it is hypothesized that the reason is (1) aFP induced thermal denaturation of the tissue which up-regulated chemokines, cytokines, and released TAA (11) and DAMPs (34) including expression of HSP70, HSP90, CALR. (2) The chemokines, cytokines and DAMPs recruited innate immune cells (45, 46) and they expressed TAA to T cells via interaction of MHC/TCR. The TCR signaling from the interaction under presence of II-1 (Table 3) stimulated OX40 expression on the T cell (28). (3) Then OX40 agonist induced T cells' proliferation, activation, and effector function leading to increase number of granzyme B+CD8+ T cells. (4) PD-1 inhibitor blocked the PD-1/PD-L1 axis leading to prevent inactivation of CD8+ T cells by PD-L1 expressing on tumor membrane. Then tumors were eradicated effectively. Regarding aFP+OX40 group, tumor could not be cured effectively and CD8+ T cells did not infiltrate by 5 days after treatment started. There are reports PD-1 is expressed on innate cells such as DCs and macrophage (38), expression of PD-L1 on tumor cells can be induced by exposure to both Type I and Type II interferons (35, 47-49), and PD-L1 can be secreted by tumor (50-52).

Regarding aFP+PD-1 group, though DCs increased than control group, CD8+ T cells could not increase shortly due to absence of OX40 agonist so that there were less survival mice. Double therapy such as anti-PD-1+OX40 also led to complete tumor remission in 83% of the mice in case treatment started 6 days after tumor inoculation. However, tumor volume curve shows tumor volume peak of cured mice is 2 days delayed as compared to that of the triple therapy aFP+anti-PD-1+OX40 group, meaning that the tumors shrank slower than the triple therapy group. Flow cytometry analysis showed that the total number of CD8+ T cells in lymph node and tumor in the double therapy group did not increase as compared with control group at 5 days after the treatment started. The inventors expect the reason is that TAA secretion from, and HSP70, HSP90 and CALR expression on the tumor cells and OX40 expression on the T cells due to absence of inflammatory cytokines were insufficient to induce anti-tumor immunity so that CD8+ T cells and granzyme B+CD8+ T cells were not induced effectively by 5 days after the treatment started, even though PD-1 inhibitor and OX40 agonist were administered. When treatment was initiated 12 days after tumor inoculation, 30% of the mice were cured in the double therapy group, though 80% of mice were cured in the triple therapy group leading to significant difference. Generally, there has been difficulty in initiating a protective immune response against large tumors since large tumors grow quickly and exert immune evasion such as recruiting Treg and myeloid derived suppressor cells (MDSC), secreting immune suppressor cytokine, expressing immune suppressor molecule and down-regulating MHC class I molecules for reduction of expression of the immunogenic antigens(53, 54). For successful eradication of a large tumor, induction of anti-tumor immunity as early as possible is important.

In conclusion, the inventors used an OX40 agonist in addition to aFP+anti-PD-1 double therapy. Combining double therapy with an OX40 agonist boosted the systemic immunity effectively. These effects may be mediated by release of TAA by aFP treatment, proliferation of the TAA specific CD8+ T cells induced by OX40 agonist and blocking PD-1/PD-L1 axis by anti-PD-1 inhibitor.

### Materials and Methods

### Cell Lines

CT26WT murine colon and 4T1 murine mammary carcinoma cell lines (ATCC, Mannassas, VA) were cultured in RPMI Medium supplemented with 10% heat-inactivated fetal bovine serum, penicillin (100 U/mL) and streptomycin (100 mg/mL) (all from Sigma-Aldrich, Natick, MA) at 37° C. in 5% CO₂. Culturing was performed in 75 cm² flasks (Falcon, Invitrogen, Carlsbad, CA).

### Animals

Six-week-old female BALB/c mice (Charles River Laboratories, Boston, MA) were used for the study. The care and handling of the animals were done in accordance with a protocol approved by the Subcommittee on Research Animal Care (IACUC) at Massachusetts General Hospital (MGH).

### Animal Tumor Model

Mice were inoculated bilaterally with 3.5×10⁵ CT26WT subcutaneously into the depilated thigh, after being anesthetized through intraperitoneal injection of a cocktail of ketamine (90 mg/kg) and xylazine (10 mg/kg). Anti-PD-1 blocking and/or anti-OX40 agonist antibodies (29F.1A12 and OX-86 respectively; BioXCell, West Lebanon, NH) were administered intraperitoneally at a dose of 200 μg per mouse on days 6, 8, 10, 12, and 14, or 12, 14, 16, 18 and 20 after tumor cell inoculation (FIG. 10). Anti-CD8 depletion antibodies (2.43; BioXCell, West Lebanon, NH) were administered intraperitoneally at a dose of 200 μg per mouse every 3 days from one day before tumor inoculation to removal of mice as endpoint. The tumor volume was determined at least 2 times per week by measuring the longest dimension and orthogonal dimension of the tumor with vernier calipers. Tumor volumes were calculated according to the formula volume= [symbol] 4π/3×[(a+b)/4]³, where a and b represent the long and short axis lengths, respectively. If the tumor volume exceeded 500 mm³ or showed severe ulceration, the mouse was removed from the study as endpoint.

### Fractional CO₂ Laser Irradiation

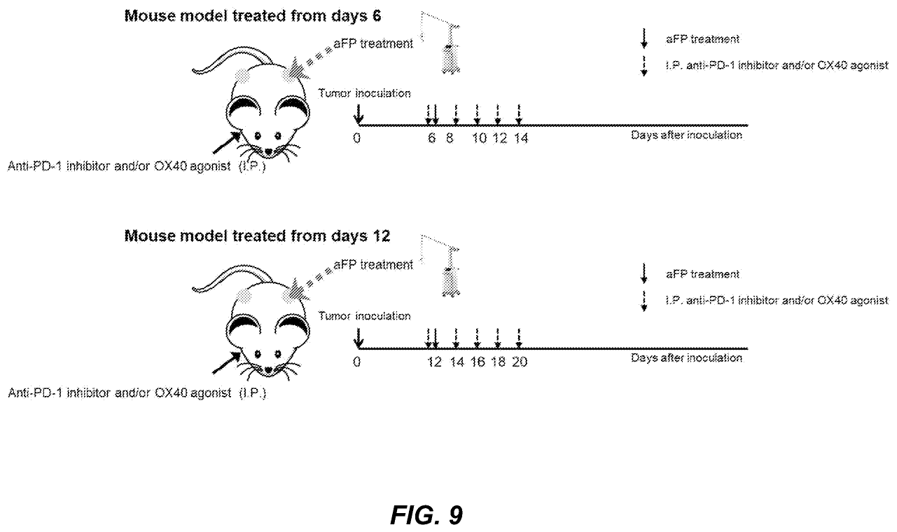

Ablative fractional laser exposure was performed on the tumor site at day 6 or 12 after tumor cell inoculation (FIG. 9). At that time, the tumors have reached a typical diameter of about 4 or 7 mm. Exposures were performed with an Ultrapulse Encore CO₂ laser (Lumenis Inc, Yokneam, Israel). A single aFP treatment was performed within a treatment area of 5×5 mm, with a pulse energy of 100 mJ, at a nominal density of 5% (i.e., 5% within the treatment area surface was irradiated), and a pulse repetition frequency of 300 Hz. No skin cooling was applied and the anesthesia was performed by intraperitoneal injection of a cocktail of ketamine (90 mg/kg) and xylazine (10 mg/kg).

### Rechallenge

Mice surviving 90 days after tumor inoculation were rechallenged with matched tumor cells (3.5×10⁵ CT26WT) subcutaneously at the contralateral (right) leg from previously aFP-treated side. Age-matched naive mice were inoculated with the same number of the cells in the right leg as the controls. The inoculated mice were monitored for another 60 days to confirm tumorigenesis.

### Flow Cytometry Analysis

To confirm whether CD3+ and CD8+ lymphocyte, regulatory T cell (Treg), and Dendritic cells (DCs) numbers were affected by the treatments, flow cytometry analysis was performed. For leukocyte isolation from tumor, fresh CT26WT tumors were dissociated mechanically filtering through 70 μm strainer on 6 well culture plate with DNaseI (10 μg/ml, Roche; Nutley, NJ) and collagenase (10 mg/ml, Life technologies) and incubated 60 minutes at 37° C. The dissociated cells were stained with anti-CD45 (30-F11), CD3 (145-2C11), CD4 (RM4-5), CD8a (53-6.7), CD103 (2E7), OX40 (OX-86; all from eBioscience; Santa Clare, CA), CD11c (HL3; BD Biosciences; San Jose, CA), IA/IE (M5/11415.2), CCR7 (4B12), XCR1 (ZET; all from BioLegend; San Diego; CA) antibodies for 30 minutes at 4° C. after they were blocked at 4° C. for 15 minutes using anti-CD16/CD32 antibody (93). Following staining for surface markers, cells were fixed and permeabilized by Foxp3/Transcription Factor Staining Buffer Set (eBioscience) according to the manufacturer's instructions at room temperature for 30 minutes. After the permeabilization the cells were stained with anti-Foxp3 (FJK-16s), Ki67(SolA15), or granzyme B (NGZB; all from eBioscience) antibody at 4° C. overnight. The next day the stained cells were analyzed on Fortessa X-20 (BD Biosciences).

### Statistics

All experiments were repeated at least once. All statics analyses were performed with GraphPad Prism 9.0 (GraphPad Software). All values are expressed as the mean±SD. Flow cytometric results were compared with one-way ANOVA. Survival analysis was performed using the Kaplan-Meier method and a log-rank test. Values of P<0.05 were considered statistically significant.

### REFERENCES

1. Manstein D, Herron G S, Sink R K, Tanner H, Anderson R R. Fractional photothermolysis: a new concept for cutaneous remodeling using microscopic patterns of thermal injury. Lasers Surg Med. 2004; 34(5):426-38.
2. Tierney E P, Hanke C W, Petersen J. Ablative fractionated CO2 laser treatment of photoaging: a clinical and histologic study. Dermatol Surg. 2012; 38(11):1777-89.
3. Prignano F, Campolmi P, Bonan P, Ricceri F, Cannarozzo G, Troiano M, et al. Fractional CO₂ laser: a novel therapeutic device upon photobiomodulation of tissue remodeling and cytokine pathway of tissue repair. Dermatol Ther. 2009; 22 Suppl 1:S8-15.
4. Kim H W, Chang S E, Kim J E, Ko J Y, Ro Y S. The safe delivery of fractional ablative carbon dioxide laser treatment for acne scars in Asian patients receiving oral isotretinoin. Dermatol Surg. 2014; 40(12):1361-6.
5. Trelles M A, Velez M, Gold M H. The treatment of melasma with topical creams alone, CO₂ fractional ablative resurfacing alone, or a combination of the two: a comparative study. J Drugs Dermatol. 2010; 9(4):315-22.
6. Khan M H, Sink R K, Manstein D, Eimerl D, Anderson R R. Intradermally focused infrared laser pulses: thermal effects at defined tissue depths. Lasers Surg Med. 2005; 36(4):270-80.
7. Hantash B M, Bedi V P, Chan K F, Zachary C B. Ex vivo histological characterization of a novel ablative fractional resurfacing device. Lasers Surg Med. 2007; 39(2):87-95.
8. Tierney E P, Kouba D J, Hanke C W. Review of fractional photothermolysis: treatment indications and efficacy. Dermatol Surg. 2009; 35(10):1445-61.
9. Wenande E, Erlendsson A M, Haedersdal M. Opportunities for laser-assisted drug delivery in the treatment of cutaneous disorders. Semin Cutan Med Surg. 2017; 36(4):192-201.
10. Kawakubo M, Demehri S, Manstein D. Fractional laser exposure induces neutrophil infiltration (N1 phenotype) into the tumor and stimulates systemic anti-tumor immune response. PLoS One. 2017; 12(9):e0184852.
11. Kawakubo M, Cunningham T J, Demehri S, Manstein D. Fractional Laser Releases Tumor-Associated Antigens in Poorly Immunogenic Tumor and Induces Systemic Immunity. Sci Rep. 2017; 7(1):12751.
12. Song J, So T, Cheng M, Tang X, Croft M. Sustained survivin expression from OX40 costimulatory signals drives T cell clonal expansion. Immunity. 2005; 22(5):621-31.
13. Weinberg A D, Rivera M M, Prell R, Morris A, Ramstad T, Vetto J T, et al. Engagement of the OX-40 receptor in vivo enhances antitumor immunity. J Immunol. 2000; 164(4):2160-9.
14. Biagi E, Dotti G, Yvon E, Lee E, Pule M, Vigouroux S, et al. Molecular transfer of CD40 and OX40 ligands to leukemic human B cells induces expansion of autologous tumor-reactive cytotoxic T lymphocytes. Blood. 2005; 105(6):2436-42.
15. Gramaglia I, Weinberg A D, Lemon M, Croft M. Ox-40 ligand: a potent costimulatory molecule for sustaining primary CD4 T cell responses. J Immunol. 1998; 161(12):6510-7.
16. Dannull J, Nair S, Su Z, Boczkowski D, DeBeck C, Yang B, et al Enhancing the immunostimulatory function of dendritic cells by transfection with mRNA encoding OX40 ligand. Blood. 2005; 105(8):3206-13.
17. Murata S, Ladle B H, Kim P S, Lutz E R, Wolpoe M E, Ivie S E, et al. OX40 costimulation synergizes with G M-CSF whole-cell vaccination to overcome established CD8+ T cell tolerance to an endogenous tumor antigen. J Immunol. 2006; 176(2):974-83.
18. Kitamura N, Murata S, Ueki T, Mekata E, Reilly R T, Jaffee E M, et al. OX40 costimulation can abrogate Foxp3+ regulatory T cell-mediated suppression of antitumor immunity. Int J Cancer. 2009; 125(3):630-8.
19. Piconese S, Valzasina B, Colombo M P. OX40 triggering blocks suppression by regulatory T cells and facilitates tumor rejection. J Exp Med. 2008; 205(4):825-39.
20. Valzasina B, Guiducci C, Dislich H, Killeen N, Weinberg A D, Colombo M P. Triggering of OX40 (CD134) on CD4(+)CD25+ T cells blocks their inhibitory activity: a novel regulatory role for OX40 and its comparison with GITR. Blood. 2005; 105(7):2845-51.
21. Vu M D, Xiao X, Gao W, Degauque N, Chen M, Kroemer A, et al. OX40 costimulation turns off Foxp3+Tregs. Blood. 2007; 110(7):2501-10.
22. Broz M L, Binnewies M, Boldajipour B, Nelson A E, Pollack J L, Erle D J, et al. Dissecting the tumor myeloid compartment reveals rare activating antigen-presenting cells critical for T cell immunity. Cancer Cell. 2014; 26(5):638-52.
23. Roberts E W, Broz M L, Binnewies M, Headley M B, Nelson A E, Wolf D M, et al. Critical Role for CD103(+)/CD141(+) Dendritic Cells Bearing CCR7 for Tumor Antigen Trafficking and Priming of T Cell Immunity in Melanoma. Cancer Cell. 2016; 30(2):324-36.
24. Eisenbarth S C. Dendritic cell subsets in T cell programming: location dictates function. Nat Rev Immunol. 2019; 19(2):89-103.
25. Dorner B G, Dorner M B, Zhou X, Opitz C, Mora A, Giittler S, et al. Selective expression of the chemokine receptor XCR1 on cross-presenting dendritic cells determines cooperation with CD8+ T cells. Immunity. 2009; 31(5):823-33.
26. Crozat K, Tamoutounour S, Vu Manh T P, Fossum E, Luche H, Ardouin L, et al. Cutting edge: expression of XCR1 defines mouse lymphoid-tissue resident and migratory dendritic cells of the CD8α+ type. J Immunol. 2011; 187(9):4411-5.
27. Ahn E, Araki K, Hashimoto M, Li W, Riley J L, Cheung J, et al. Role of PD-1 during effector CD8 T cell differentiation. Proc Natl Acad Sci USA. 2018; 115(18):4749-54.
28. Croft M, So T, Duan W, Soroosh P. The significance of OX40 and OX40L to T-cell biology and immune disease. Immunol Rev. 2009; 229(1):173-91.
29. Shevtsov M, Multhoff G. Heat Shock Protein-Peptide and HSP-Based Immunotherapies for the Treatment of Cancer. Front Immunol. 2016; 7:171.
30. Obeid M, Tesniere A, Ghiringhelli F, Fimia G M, Apetoh L, Perfettini J L, et al. Calreticulin exposure dictates the immunogenicity of cancer cell death. Nat Med. 2007; 13(1):54-61.
31. Mroz P, Vatansever F, Muchowicz A, Hamblin M R. Photodynamic therapy of murine mastocytoma induces specific immune responses against the cancer/testis antigen N A. Cancer Res. 2013; 73(21): 6462-70.
32. Mroz P, Szokalska A, Wu M X, Hamblin M R. Photodynamic therapy of tumors can lead to development of systemic antigen-specific immune response. PLoS One. 2010; 5(12):e15194.
33. Huang A Y, Gulden P H, Woods A S, Thomas M C, Tong C D, Wang W, et al. The immunodominant major histocompatibility complex class I-restricted antigen of a murine colon tumor derives from an endogenous retroviral gene product. Proc Natl Acad Sci USA. 1996; 93(18):9730-5.
34. Hazeldine J, Hampson P, Lord J M. The diagnostic and prognostic value of systems biology research in major traumatic and thermal injury: a review. Burns Trauma. 2016; 4:33.
35. Iwai Y, Ishida M, Tanaka Y, Okazaki T, Honjo T, Minato N. Involvement of PD-L1 on tumor cells in the escape from host immune system and tumor immunotherapy by PD-L1 blockade. Proc Natl Acad Sci USA. 2002; 99(19):12293-7.
36. Fanoni D, Tavecchio S, Recalcati S, Balice Y, Venegoni L, Fiorani R, et al. New monoclonal antibodies against B-cell antigens: possible new strategies for diagnosis of primary cutaneous B-cell lymphomas. Immunol Lett. 2011; 134(2):157-60.
37. Terme M, Ullrich E, Aymeric L, Meinhardt K, Desbois M, Delahaye N, et al. IL-18 induces PD-1-dependent immunosuppression in cancer. Cancer Res. 2011; 71(16):5393-9.
38. Ostrand-Rosenberg S, Horn L A, Haile S T. The programmed death-1 immune-suppressive pathway: barrier to antitumor immunity. J Immunol. 2014; 193(8):3835-41.
39. Peng Q, Qiu X, Zhang Z, Zhang S, Zhang Y, Liang Y, et al. PD-L1 on dendritic cells attenuates T cell activation and regulates response to immune checkpoint blockade. Nat Commun. 2020; 11(1):4835.
40. Zou W, Chen L. Inhibitory B7-family molecules in the tumour microenvironment. Nat Rev Immunol. 2008; 8(6):467-77.
41. Dong H, Strome S E, Salomao D R, Tamura H, Hirano F, Flies D B, et al. Tumor-associated B7-H1 promotes T-cell apoptosis: a potential mechanism of immune evasion. Nat Med. 2002; 8(8):793-800.
42. Konishi J, Yamazaki K, Azuma M, Kinoshita I, Dosaka-Akita H, Nishimura M. B7-H1 expression on non-small cell lung cancer cells and its relationship with tumor-infiltrating lymphocytes and their PD-1 expression. Clin Cancer Res. 2004; 10(15):5094-100.
43. Pan P Y, Zang Y, Weber K, Meseck M L, Chen S H. OX40 ligation enhances primary and memory cytotoxic T lymphocyte responses in an immunotherapy for hepatic colon metastases. Mol Ther. 2002; 6(4):528-36.
44. Linch S N, McNamara M J, Redmond W L. OX40 Agonists and Combination Immunotherapy: Putting the Pedal to the Metal. Front Oncol. 2015; 5:34.
45. Krysko D V, Garg A D, Kaczmarek A, Krysko O, Agostinis P, Vandenabeele P. Immunogenic cell death and DAMPs in cancer therapy. Nat Rev Cancer. 2012; 12(12):860-75.
46. Zelenay S, Reis e Sousa C. Adaptive immunity after cell death. Trends Immunol. 2013; 34(7): 329-35.
47. Loke P, Allison J P. PD-L1 and PD-L2 are differentially regulated by Th1 and Th2 cells. Proc Natl Acad Sci USA. 2003; 100(9):5336-41.
48. Topalian S L, Drake C G, Pardoll D M. Targeting the PD-1/B7-H1(PD-L1) pathway to activate anti tumor immunity. Curr Opin Immunol. 2012; 24(2):207-12.
49. Eppihimer M J, Gunn J, Freeman G J, Greenfield E A, Chernova T, Erickson J, et al. Expression and regulation of the PD-L1 immunoinhibitory molecule on microvascular endothelial cells. Microcirculation. 2002; 9(2):133-45.
50. Keir M E, Butte M J, Freeman G J, Sharpe A H. PD-1 and its ligands in tolerance and immunity. Annu Rev Immunol. 2008; 26:677-704.
51. Latchman Y, Wood C R, Chernova T, Chaudhary D, Borde M, Chernova I, et al. P D-L2 is a second ligand for PD-1 and inhibits T cell activation. Nat Immunol. 2001; 2(3):261-8.
52. Frigola X, Inman B A, Lohse C M, Krco C J, Cheville J C, Thompson R H, et al. Identification of a soluble form of B7-H1 that retains immunosuppressive activity and is associated with aggressive renal cell carcinoma. Clin Cancer Res. 2011; 17(7):1915-23.
53. Vinay D S, Ryan E P, Pawelec G, Talib W H, Stagg J, Elkord E, et al. Immune evasion in cancer: Mechanistic basis and therapeutic strategies. Semin Cancer Biol. 2015; 35 Suppl:S185-s98.
54. Spranger S. Mechanisms of tumor escape in the context of the T-cell-inflamed and the non-T-cell-inflamed tumor microenvironment. Int Immunol. 2016; 28(8):383-91.

Any reference, patent or patent application publication disclosed in the present application is incorporated by reference herein in its entirety.

While the invention has been described with reference to preferred embodiments, those skilled in the art will appreciate that certain substitutions, alterations and/or omissions may be made to the embodiments without departing from the spirit of the invention. Accordingly, the foregoing description is meant to be exemplary only, and should not limit the scope of the invention.

1. A method for inducing an immune response in a subject in need thereof, the method comprising:
(a) administering an inhibitor of a blocking checkpoint molecule and an agonist of a stimulative checkpoint molecule to a subject in need thereof, and
(b) treating a tissue of the subject with energy to induce fractional tissue damage,
wherein an immune response is increased compared to the immune response produced by the inhibitor of the blocking checkpoint molecule and the agonist of the stimulative checkpoint molecule in the absence of the fractional tissue damage.

2. The method of claim 1, wherein the fractional tissue damage induces CD8+ T cell recruitment and/or activation.

3. The method of claim 1 or 2, wherein the energy that induces fractional tissue damage is selected from laser energy, ionizing radiation, ultrasound, and radio frequency energy.

4. The method of any one of claims 1-3, wherein the blocking checkpoint molecule is selected from the group consisting of: PD-1, PD-L1, CTLA4, B7-H3, B7-H4, VISTA, TMIGD2, B7-H7, BTLA, HVEM, CD160, LAG3, TIGIT, CD96, CD155, TIM-3, Galectin-9, Adenosine, Adenosine A2a receptor, IDO, TDO, CEACAM1, SIRP alpha, CD47, CD200R and CD200.

5. The method of any one of claims 1-4, wherein the stimulative checkpoint molecule is selected from the group consisting of: OX40, 4-1BB, GITR, CD28, ICOS, LIGHT, CD27, DNAM-1, 2B4, DC-SIGN, DR3, and CD40.

6. The method of any one of claims 1-5, wherein the blocking checkpoint molecule is PD-1 and the stimulative checkpoint molecule is OX40.

7. The method of claim 6, wherein the PD-1 inhibitor and/or the OX40 agonist comprises an antibody.

8. The method of any one of claims 1-7, wherein the cancer is colon cancer, lung cancer, melanoma, or breast cancer.

9. The method of claim 8, wherein the laser energy is emitted from a fractional CO₂ laser.

10. The method of any one of claims 1-9, wherein the immune response comprises a local and/or systemic response.

11. A method for inducing an anti-tumor immune response in a subject in need thereof, the method comprising:
(a) administering an inhibitor of a blocking checkpoint molecule and an agonist of a stimulative checkpoint molecule to a subject having cancer, and
(b) treating tumor tissue of the subject with energy to induce fractional tissue damage,
wherein an anti-tumor immune response is increased compared to the anti-tumor immune response produced by the inhibitor of the blocking checkpoint molecule and the agonist of the stimulative checkpoint molecule in the absence of the fractional tissue damage.

12. A method for treating cancer in a subject in need thereof, the method comprising:
(a) administering an inhibitor of a blocking checkpoint molecule, and an agonist of a stimulative checkpoint molecule to a subject having cancer, and
(b) treating tumor tissue of the subject with energy to induce fractional tissue damage, thereby treating cancer in the subject.

13. The method of claim 11 or claim 12, wherein the energy that induces fractional tissue damage is selected from laser energy, ionizing radiation, ultrasound, and radio frequency energy.

14. The method of any one of claims 11-13, wherein the energy that induces fractional tissue damage is laser energy.

15. The method of any one of claims 11-14, wherein the blocking checkpoint molecule is selected from the group consisting of: PD-1, PD-L1, CTLA4, B7-H3, B7-H4, VISTA, TMIGD2, B7-H7, BTLA, HVEM, CD160, LAG3, TIGIT, CD96, CD155, TIM-3, Galectin-9, Adenosine, Adenosine A2a receptor, IDO, TDO, CEACAM1, SIRP alpha, CD47, CD200R and CD200.

16. The method of any one of claims 11-15, wherein the stimulative checkpoint molecule is selected from the group consisting of: OX40, 4-1BB, GITR, CD28, ICOS, LIGHT, CD27, DNAM-1, 2B4, DC-SIGN, DR3, and CD40.

17. The method of any one of claims 11-16, wherein the blocking checkpoint molecule is PD-1 and the stimulative checkpoint molecule is OX40.

18. The method of claim 17, wherein the PD-1 inhibitor and/or the OX40 agonist comprises an antibody.

19. The method of any one of claims 11-18, wherein the cancer is colon cancer, lung cancer, melanoma, or breast cancer.

20. The method of claim 19, wherein the laser energy is emitted from a fractional CO₂ laser.

21. The method of any one of claims 11-20, wherein the anti-tumor immune response or the treatment of cancer comprises induction of CD8+ T cells.

22. The method of any one of claims 11-21, wherein the anti-tumor immune response comprises a systemic response.

23. The method of claim 22, wherein the anti-tumor immune response induces an abscopal effect against a tumor that is not treated with the fractional laser to induce fractional tissue damage.

24. The method of any one of claims 11-23, wherein the anti-tumor immune response prevents or reduces the likelihood of cancer recurrence.

25. The method of any one of claims 11-24, wherein the anti-tumor immune response increases progression-free survival, reduces the size of one or more tumors, and/or increases overall response rate.

26. A method for inducing an anti-tumor immune response in a subject in need thereof, the method comprising:
(a) administering an OX40 agonist to a subject having cancer, and
(b) treating tumor tissue of the subject with energy to induce fractional tissue damage,
Wherein the anti-tumor immune response is increased compared to the anti-tumor immune response produced by the OX40 agonist of in the absence of the fractional tissue damage.

27. A method for treating cancer in a subject, the method comprising:
(a) administering an OX40 agonist to a subject having cancer, and
(b) treating tumor tissue of the subject with energy to induce fractional tissue damage,
thereby treating cancer in the subject.

28. The method of claim 26 or 27, further comprising administering an inhibitor of a blocking checkpoint molecule selected from the group consisting of: PD-1, PD-L1, CTLA4, B7-H3, B7-H4, VISTA, TMIGD2, B7-H7, BTLA, HVEM, CD160, LAG3, TIGIT, CD96, CD155, TIM-3, Galectin-9, Adenosine, Adenosine A2a receptor, IDO, TDO, CEACAM1, SIRP alpha, CD47, CD200R and CD200.

29. The method of any one of claims 26-28, wherein the blocking checkpoint molecule is PD-1.

30. The method of claim 29, wherein the PD-1 inhibitor and/or the OX4 agonist comprises an antibody.

31. The method of any one of claims 26-30, wherein the cancer is colon cancer, lung cancer, melanoma, or breast cancer.

32. The method of any one of claims 26-31, wherein the energy that induces fractional tissue damage is selected from laser energy, ionizing radiation, ultrasound, and radio frequency energy.

33. The method of any one of claims 26-32, wherein the fractional laser comprises a fractional CO₂ laser.

34. The method of any one of claims 26-33, wherein the anti-tumor immune response or treatment of cancer comprises induction of CD8+ T cells, increase in number of CD8+ T cells, or activation of CD8+ T cells.

35. The method of any one of claims 26-34, wherein the anti-tumor immune response or treatment of cancer comprises a systemic response.

36. The method of any one of claims 26-35, wherein the anti-tumor immune response or treatment of cancer induces abscopal treatment of a tumor that is not treated with energy emitted from the fractional laser to induce fractional tissue damage.

37. The method of any one of claims 26-36, wherein the anti-tumor immune response prevents or reduces the likelihood of cancer recurrence.

38. The method of any one of claims 26-37, wherein the anti-tumor immune response increases progression-free survival, reduces the size of one or more tumors, and/or increases overall response rate.

39. A system for inducing an anti-tumor immune response or treating cancer in a subject, the system comprising: a device configured to induce fractional tissue damage and means for administering an inhibitor of a blocking checkpoint molecule, and an agonist of a stimulative checkpoint molecule.

40. The system of claim 39, wherein the device configured to induce fractional tissue damage comprises a fractional laser, radiofrequency energy, or focused ultrasound.

41. The system of claim 40, wherein the fractional laser is an ablative fractional laser.

42. The system of claim 41, wherein the fractional laser is a fractional CO₂ laser.

43. A method of inducing pyroptosis in tumor cells in a subject, the method comprising:
(a) treating tumor tissue of a subject with energy to induce fractional tissue damage; and
(b) administering an inhibitor of a blocking checkpoint molecule and an agonist of a stimulative checkpoint molecule to the subject,
wherein pyroptosis is induced in tumor cells in the subject.

44. The method of claim 43, wherein pyroptosis is induced at a site separate from the tumor tissue treated with energy to induce fractional tissue damage.

45. A method for inducing an anti-tumor immune response in a subject in need thereof, the method comprising:
(a) administering one or more agents to a subject having cancer, and
(b) treating tumor tissue of the subject with energy to induce fractional tissue damage,
wherein the number of CD8+ T cells or activated CD8+ T cells is increased in the tumor compared to the anti-tumor immune response produced by the one or more agents in the absence of the fractional tissue damage.

46. A method for treating cancer in a subject, the method comprising:
(a) administering one or more agents to a subject having cancer, and
(b) treating tumor tissue of the subject with energy to induce fractional tissue damage,
wherein the number of CD8+ T cells or activated CD8+ T cells is increased in the tumor,
thereby treating the cancer in the subject.
